SEQUENCE LISTING (197 sequences)

SEQ ID NO: 1 - protein, 330 aa, Homo sapiens [sequence omitted]

SEQ ID NO: 2 - protein, 105 aa, Homo sapiens
  IgG constant region light (CL)
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 3 - protein, 118 aa, Artificial Sequence
  Antibody clone 1A01-VH variable region heavy (VH)
EVQLLESGGGLVQPGGSLRLSCAASGFTFSDYYMNWIRQTPGKGLEWVSLIGWDGGSTYYADSVKGRFTISRDNSENTLYLQMNSLRAEDTAVYYCARAYSGYELDYWGQGTLVTVSS

SEQ ID NO: 4 - protein, 117 aa, Artificial Sequence
  Antibody clone 1B07 variable region heavy (VH)
EVQLLESGGGLVQPGGSLRLSCAASGFTFSSYGMHWVRQAPGKGLEWVAFTRYDGSNKYYADSVRGRFTISRDNSKNTLYLQMNSLRAEDTAVYYCARENIDAFDVWGQGTLVTVSS

SEQ ID NO: 5 - protein, 122 aa, Artificial Sequence
  Antibody clone 1C04 variable region heavy (VH)
EVQLLESGGGLVQPGGSLRLSCAASGFTFSSYAMSWVRQAPGKGLEWVSSISDSGAGRYYADSVEGRFTISRDNSKNTLYLQMNSLRAEDTAVYYCARTHDSGELLDAFDIWGQGTLVTVSS

SEQ ID NO: 6 - protein, 124 aa, Artificial Sequence
  Antibody clone 1E05 variable region heavy (VH)
EVQLLESGGGLVQPGGSLRLSCAASGFTFSTYAMNWVRQVPGKGLEWVAVISYDGSNKNYVDSVKGRFTISRDNSKNTLYLQMNSLRAEDTAVYYCARNFDNSGYAIPDAFDIWGQGTLVTVSS

SEQ ID NO: 7 - protein, 121 aa, Artificial Sequence
  Antibody clone 2A09 variable region heavy (VH)
EVQLLESGGGLVQPGGSLRLSCAASGFTFSNAWMSWVRQAPGKGLEWVAYISRDADITHYPASVKGRFTISRDNSKNTLYLQMNSLRAEDTAVYYCTTGFDYAGDDAFDIWGQGTLVTVSS

SEQ ID NO: 8 - protein, 119 aa, Artificial Sequence
  Antibody clone 2B08 variable region heavy (VH)
EVQLLESGGGLVQPGGSLRLSCAASGFTFSDYYMSWVRQAPGKGLEWVALIGHDGNNKYYLDSLEGRFTISRDNSKNTLYLQMNSLRAEDTAVYYCARATDSGYDLLYWGQGTLVTVSS

SEQ ID NO: 9 - protein, 117 aa, Artificial Sequence
  Antibody clone 2E08 variable region heavy (VH)
EVQLLESGGGLVQPGGSLRLSCAASGFTFSDYYMSWIRQAPGKGLEWVSAIGFSDDNTYYADSVKGRFTISRDNSKNTLYLQMNSLRAEDTAVYYCAGGDGSGWSFWGQGTLVTVSS

SEQ ID NO: 10 - protein, 117 aa, Artificial Sequence
  Antibody clone 5C04 variable region heavy (VH)
EVQLLESGGGLVQPGGSLRLSCAASGFTFSNYGMHWVRQAPGKGLEWVAVISYDGSNKYYADSVKGRFTISRDNSKNTLYLQMNSLRAEDTAVYYCAREWRDAFDIWGQGTLVTVSS

SEQ ID NO: 11 - protein, 117 aa, Artificial Sequence
  Antibody clone 5C05 variable region heavy (VH)
EVQLLESGGGLVQPGGSLRLSCAASGFTFSTYGMHWVRQAPGKGLEWVAVISYDGSNKYYADSVKGRFTISRDNSKNTLYLQMNSLRAEDTAVYYCARENFDAFDVWGQGTLVTVSS

SEQ ID NO: 12 - protein, 117 aa, Artificial Sequence
  Antibody clone 5D07 variable region heavy (VH)
EVQLLESGGGLVQPGGSLRLSCAASGFTFSTYGMHWVRQAPGKGLEWVAVIAYDGSKKDYADSVKGRFTISRDNSKNTLYLQMNSLRAEDTAVYYCAREYRDAFDIWGQGTLVTVSS

SEQ ID NO: 13 - protein, 117 aa, Artificial Sequence
  Antibody clone 5E12 variable region heavy (VH)
EVQLLESGGGLVQPGGSLRLSCAASGFTFSSYGMHWVRQAPGKGLEWVAVISYDGINKDYADSMKGRFTISRDNSKNTLYLQMNSLRAEDTAVYYCARERKDAFDIWGQGTLVTVSS

SEQ ID NO: 14 - protein, 117 aa, Artificial Sequence
  Antibody clone 5G08 variable region heavy (VH)
EVQLLESGGGLVQPGGSLRLSCAASGFTFNNYGMHWVRQAPGKGLEWVAVISYDGSNRYYADSVKGRFTMSRDNSKNTLYLQMNSLRAEDTAVYYCARDRWNGMDVWGQGTLVTVSS

SEQ ID NO: 15 - protein, 119 aa, Artificial Sequence
  Antibody clone 5H06 variable region heavy (VH)
EVQLLESGGGLVQPGGSLRLSCAASGFTFSSYGMHWVRQAPGKGLEWVAVISYDGSDTAYADSVKGRFTISRDNSKNTLYLQMNSLRAEDTAVYYCARDHSVIGAFDIWGQGTLVTVSS

SEQ ID NO: 16 - protein, 119 aa, Artificial Sequence
  Antibody clone 6A09 variable region heavy (VH)
EVQLLESGGGLVQPGGSLRLSCAASGFTFSSYGMHWVRQAPGKGLEWVAVTSYDGNTKYYANSVKGRFTISRDNSKNTLYLQMNSLRAEDTAVYYCAREDCGGDCFDYWGQGTLVTVSS

SEQ ID NO: 17 - protein, 118 aa, Artificial Sequence
  Antibody clone 6B01 variable region heavy (VH)
EVQLLESGGGLVQPGGSLRLSCAASGFTFSNYGMHWVRQAPGKGLEWVAVISYDGSNKYYADSVKGRFTISRDNSKNTLYLQMNSLRAEDTAVYYCARDQLGEAFDIWGQGTLVTVSS

SEQ ID NO: 18 - protein, 117 aa, Artificial Sequence
  Antibody clone 6C11 variable region heavy (VH)
EVQLLESGGGLVQPGGSLRLSCAASGFTFDDYGMSWVRQAPGKGLEWVSAISGSGSSTYYADSVKGRFTISRDNSKNTLYLQMNSLRAEDTAVYYCAGGDIDYFDYWGQGTLVTVSS

SEQ ID NO: 19 - protein, 117 aa, Artificial Sequence
  Antibody clone 6C12 variable region heavy (VH)
EVQLLESGGGLVQPGGSLRLSCAASGFTFSSYGMHWVRQAPGKGLEWVAVISYDGSNKYYADSVKGRFTISRDNSKNTLYLQMNSLRAEDTAVYYCARERRDAFDIWGQGTLVTVSS

SEQ ID NO: 20 - protein, 119 aa, Artificial Sequence
  Antibody clone 6D01 variable region heavy (VH)
EVQLLESGGGLVQPGGSLRLSCAASGFTFSSYGMHWVRQAPGKGLEWVAVISYDGSNKYYADSVKGRFTISRDNSKNTLYLQMNSLRAEDTAMYYCARDHSAAGYFDYWGQGTLVTVSS

SEQ ID NO: 21 - protein, 119 aa, Artificial Sequence
  Antibody clone 6G03 variable region heavy (VH)
EVQLLESGGGLVQPGGSLRLSCAASGFTFGSYGMHWVRQAPGKGLEWVSGISWDSAIIDYAGSVKGRFTISRDNSKNTLYLQMNSLRAEDTAVYYCAKDEAAAGAFDIWGQGTLVTVSS

SEQ ID NO: 22 - protein, 121 aa, Artificial Sequence
  Antibody clone 6G08 variable region heavy (VH)
EVQLLESGGGLVQPGGSLRLSCAASGFTLSSYGISWVRQAPGKGLEWVSGISGSGGNTYYADSVKGRFTISRDNSKNTLYLQMNSLRAEDTAVYYCASSVGAYANDAFDIWGQGTLVTVSS

SEQ ID NO: 23 - protein, 117 aa, Artificial Sequence
  Antibody clone 6G11 variable region heavy (VH)
EVQLLESGGGLVQPGGSLRLSCAASGFTFSSYGMHWVRQAPGKGLEWMAVISYDGSNKYYADSVKGRFTISRDNSKNTLYLQMNSLRAEDTAVYYCARELYDAFDIWGQGTLVTVSS

SEQ ID NO: 24 - protein, 117 aa, Artificial Sequence
  Antibody clone 6H08 variable region heavy (VH)
EVQLLESGGGLVQPGGSLRLSCAASGFTFNNYGMHWVRQAPGKGLEWVAVISYDGSNKYYADSVKGRFTISKDNSKNTLYLQMNSLRAEDTAVYYCAREYKDAFDIWGQGTLVTVSS

SEQ ID NO: 25 - protein, 118 aa, Artificial Sequence
  Antibody clone 7C07 variable region heavy (VH)
EVQLLESGGGLVQPGGSLRLSCAASGFTFSSYGMHWVRQAPGKGLEWVAVISYDGSNKYYADSVKGRFTISRDNSQNTLYLQMNSLRAEDTAVYYCAREFGYIILDYWGQGTLVTVSS

SEQ ID NO: 26 - protein, 117 aa, Artificial Sequence
  Antibody clone 4B02 variable region heavy (VH)
EVQLLESGGGLVQPGGSLRLSCAASGFTFSNHGMHWVRQAPGKGLEWVAVISYDGTNKYYADSVRGRFTISRDNSKNTLYLQMNSLRAEDTAVYYCARETWDAFDVWGQGTLVTVSS

SEQ ID NO: 27 - protein, 111 aa, Artificial Sequence
  Antibody clone 1A01 variable region light (VL)
QSVLTQPPSASGTPGQRVTISCSGSSSNIGNNAVNWYQQLPGTAPKLLIYDNNNRPSGVPDRFSGSKSGTSASLAISGLRSEDEADYYCAAWDDSLNASIFGGGTKLTVLG

SEQ ID NO: 28 - protein, 111 aa, Artificial Sequence
  Antibody clone 1B07 variable region light (VL)
QSVLTQPPSASGTPGQRVTISCSGSSSNIGNNAVNWYQQLPGTAPKLLIYDNQQRPSGVPDRFSGSKSGTSASLAISGLRSEDEADYYCEAWDDRLFGPVFGGGTKLTVLG

SEQ ID NO: 29 - protein, 111 aa, Artificial Sequence
  Antibody clone 1C04 variable region light (VL)
QSVLTQPPSASGTPGQRVTISCSGSSSNIGSNHVLWYQQLPGTAPKLLIYGNSNRPSGVPDRFSGSKSGTSASLAISGLRSEDEADYYCAAWDDSLNGWVFGGGTKLTVLG

SEQ ID NO: 30 - protein, 112 aa, Artificial Sequence
  Antibody clone 1E05 variable region light (VL)
QSVLTQPPSASGTPGQRVTISCTGSSSNIGAGYDVHWYQQLPGTAPKLLIYDNNSRPSGVPDRFSGSKSGTSASLAISGLRSEDEADYYCAAWDDSLGGPVFGGGTKLTVLG

SEQ ID NO: 31 - protein, 112 aa, Artificial Sequence
  Antibody clone 2A09 variable region light (VL)
QSVLTQPPSASGTPGQRVTISCSGSSSNIGSNAVNWYQQLPGTAPKLLIYGNSDRPSGVPDRFSGSKSGTSASLAISGLRSEDEADYYCAAWDDSLNGRWVFGGGTKLTVLG

SEQ ID NO: 32 - protein, 111 aa, Artificial Sequence
  Antibody clone 2B08 variable region light (VL)
QSVLTQPPSASGTPGQRVTISCSGSSSNIGNNAVNWYQQLPGTAPKLLIYYDDLLPSGVPDRFSGSKSGTSASLAISGLRSEDEADYYCTTWDDSLSGVVFGGGTKLTVLG

SEQ ID NO: 33 - protein, 111 aa, Artificial Sequence
  Antibody clone 2E08 variable region light (VL)
QSVLTQPPSASGTPGQRVTISCSGSSSNIGNNAVNWYQQLPGTAPKLLIYDNNKRPSGVPDRFSGSKSGTSASLAISGLRSEDEADYYCATWDDSLRGWVFGGGTKLTVLG

SEQ ID NO: 34 - protein, 113 aa, Artificial Sequence
  Antibody clone 5C04 variable region light (VL)
QSVLTQPPSASGTPGQRVTISCTGSSSNIGAGYDVHWYQQLPGTAPKLLIYSDNQRPSGVPDRFSGSKSGTSASLAISGLRSEDEADYYCAAWDDSLSGSWVFGGGTKLTVLG

SEQ ID NO: 35 - protein, 113 aa, Artificial Sequence
  Antibody clone 5C05 variable region light (VL)
QSVLTQPPSASGTPGQRVTISCTGSSSNIGAGYDVHWYQQLPGTAPKLLIYSNSQRPSGVPDRFSGSKSGTSASLAISGLRSEDEADYYCAAWDDSLNGQVVFGGGTKLTVLG

SEQ ID NO: 36 - protein, 112 aa, Artificial Sequence
  Antibody clone 5D07 variable region light (VL)
QSVLTQPPSASGTPGQRVTISCTGSSSNIGAGYDVHWYQQLPGTAPKLLIYGNSNRPSGVPDRFSGSKSGTTASLAISGLRSEDEADYYCAAWDDSVSGWMFGGGTKLTVLG

SEQ ID NO: 37 - protein, 112 aa, Artificial Sequence
  Antibody clone 5E12 variable region light (VL)
QSVLTQPPSASGTPGQRVTISCTGSSSNIGAGYDVHWYQQLPGTAPKLLIYSNNQRPSGVPDRFSGSKSGTSASLAISGLRSEDEADYYCATWDDSLNGLVFGGGTKLTVLG

SEQ ID NO: 38 - protein, 113 aa, Artificial Sequence
  Antibody clone 5G08 variable region light (VL)
QSVLTQPPSASGTPGQRVTISCSGSSSNIGAGYDVHWYQQLPGTAPKLLIYANNQRPSGVPDRFSGSKSGTSASLAISGLRSEDEADYYCAAWDDSLNGPWVFGGGTKLTVLG

SEQ ID NO: 39 - protein, 110 aa, Artificial Sequence
  Antibody clone 5H06 variable region light (VL)
QSVLTQPPSASGTPGQRVTISCSGSSSNIGSNTVNWYQQLPGTAPKLLIYDNNKRPSGVPDRFSGSKSGTSASLAISGLRSEDEADYYCSSYAGSNNVVFGGGTKLTVLG

SEQ ID NO: 40 - protein, 112 aa, Artificial Sequence
  Antibody clone 6A09 variable region light (VL)
QSVLTQPPSASGTPGQRVTISCTGSSSNIGAGYDVHWYQQLPGTAPKLLIYGNSNRPSGVPDRFSGSKSGTSASLAISGLRSEDEADYYCAAWDDSLNEGVFGGGTKLTVLG

SEQ ID NO: 41 - protein, 112 aa, Artificial Sequence
  Antibody clone 6B01 variable region light (VL)
QSVLTQPPSASGTPGQRVTISCTGSSSNIGAGYDVHWYQQLPGTAPKLLIYDNNKRPSGVPDRFSGSKSGTSASLAISGLRSEDEADYYCATWDDSLSGPVFGGGTKLTVLG

SEQ ID NO: 42 - protein, 112 aa, Artificial Sequence
  Antibody clone 6C11 variable region light (VL)
QSVLTQPPSASGTPGQRVTISCTGSSSNFGAGYDVHWYQQLPGTAPKLLIYENNKRPSGVPDRFSGSKSGTSASLAISGLRSEDEADYYCAAWDDSLNGPVFGGGTKLTVLG

SEQ ID NO: 43 - protein, 110 aa, Artificial Sequence
  Antibody clone 6C12 variable region light (VL)
QSVLTQPPSASGTPGQRVTISCTGSSSNIGAGYDVHWYQQLPGTAPKLLIYSDNQRPSGVPDRFSGSKSGTSASLAISGLRSEDEADYYCATWDSDTPVFGGGTKLTVLG

SEQ ID NO: 44 - protein, 111 aa, Artificial Sequence
  Antibody clone 6D01 variable region light (VL)
QSVLTQPPSASGTPGQRVTISCSGSSSNIGSNTVNWYQQLPGTAPKLLIYGNSIRPSGGPDRFSGSKSGTSASLAISGLRSEDEADYYCASWDDSLSSPVFGGGTKLTVLG

SEQ ID NO: 45 - protein, 113 aa, Artificial Sequence
  Antibody clone 6G03 variable region light (VL)
QSVLTQPPSASGTPGQRVTISCTGSSSNIGAGYDVHWYQQLPGTAPKLLIYGNTDRPSGVPDRFSGSKSGTSASLAISGLRSEDEADYYCAAWDDSLSGPVVFGGGTKLTVLG

SEQ ID NO: 46 - protein, 112 aa, Artificial Sequence
  Antibody clone 6G08 variable region light (VL)
QSVLTQPPSASGTPGQRVTISCTGSSSNIGAGYDVHWYQQLPGTAPKLLIYGDTNRPSGVPDRFSGSKSGTSASLAISGLRSEDEADYYCAAWDDSLNGPVFGGGTKLTVLG

SEQ ID NO: 47 - protein, 112 aa, Artificial Sequence
  Antibody clone 6G11 variable region light (VL)
QSVLTQPPSASGTPGQRVTISCTGSSSNIGAGYDVHWYQQLPGTAPKLLIYADDHRPSGVPDRFSGSKSGTSASLAISGLRSEDEADYYCASWDDSQRAVIFGGGTKLTVLG

SEQ ID NO: 48 - protein, 109 aa, Artificial Sequence
  Antibody clone 6H08 variable region light (VL)
QSVLTQPPSASGTPGQRVTISCTGSSSNIGSNTVNWYQQLPGTAPKLLIYDNNKRPSGVPDRFSGSKSGTSASLAISGLRSEDEADYYCQAWGTGIRVFGGGTKLTVLG

SEQ ID NO: 49 - protein, 111 aa, Artificial Sequence
  Antibody clone 7C07 variable region light (VL)
QSVLTQPPSASGTPGQRVTISCSGSSSNIGSNTVNWYQQLPGTAPKLLIYRDYERPSGVPDRFSGSKSGTSASLAISGLRSEDEADYYCMAWDDSLSGVVFGGGTKLTVLG

SEQ ID NO: 50 - protein, 109 aa, Artificial Sequence
  Antibody clone 4B02 variable region light (VL)
QSVLTQPPSASGTPGQRVTISCSGSSSNIGSNNANWYQQLPGTAPKLLIYDNNKRPSGVPDRFSGSKSGTSASLAISGLRSEDEADYYCQAWDSSTVVFGGGTKLTVLG

SEQ ID NO: 51 - protein, 5 aa, Artificial Sequence
  Antibody clone 1A01 CDRH1
DYYMN

SEQ ID NO: 52 - protein, 17 aa, Artificial Sequence
  Antibody clone 1A01 CDRH2
LIGWDGGSTYYADSVKG

SEQ ID NO: 53 - protein, 9 aa, Artificial Sequence
  Antibody clone 1A01 CDRH3
AYSGYELDY

SEQ ID NO: 54 - protein, 13 aa, Artificial Sequence
  Antibody clone 1A01 CDRL1
SGSSSNIGNNAVN

SEQ ID NO: 55 - protein, 7 aa, Artificial Sequence
  Antibody clone 1A01 CDRL2
DNNNRPS

SEQ ID NO: 56 - protein, 11 aa, Artificial Sequence
  Antibody clone 1A01 CDRL3
AAWDDSLNASI

SEQ ID NO: 57 - protein, 5 aa, Artificial Sequence
  Antibody clone 1B07 CDRH1
SYGMH

SEQ ID NO: 58 - protein, 17 aa, Artificial Sequence
  Antibody clone 1B07 CDRH2
FTRYDGSNKYYADSVRG

SEQ ID NO: 59 - protein, 8 aa, Artificial Sequence
  Antibody clone 1B07 CDRH3
ENIDAFDV

SEQ ID NO: 60 - protein, 13 aa, Artificial Sequence
  Antibody clone 1B07 CDRL1
SGSSSNIGNNAVN

SEQ ID NO: 61 - protein, 7 aa, Artificial Sequence
  Antibody clone 1B07 CDRL2
DNQQRPS

SEQ ID NO: 62 - protein, 9 aa, Artificial Sequence
  Antibody clone 1B07 CDRL3
WDDRLFGPV

SEQ ID NO: 63 - protein, 5 aa, Artificial Sequence
  Antibody clone 1C04 CDRH1
SYAMS

SEQ ID NO: 64 - protein, 17 aa, Artificial Sequence
  Antibody clone 1C04 CDRH2
SISDSGAGRYYADSVEG

SEQ ID NO: 65 - protein, 13 aa, Artificial Sequence
  Antibody clone 1C04 CDRH3
THDSGELLDAFDI

SEQ ID NO: 66 - protein, 13 aa, Artificial Sequence
  Antibody clone 1C04 CDRL1
SGSSSNIGSNHVL

SEQ ID NO: 67 - protein, 7 aa, Artificial Sequence
  Antibody clone 1C04 CDRL2
GNSNRPS

SEQ ID NO: 68 - protein, 11 aa, Artificial Sequence
  Antibody clone 1C04 CDRL3
AAWDDSLNGWV

SEQ ID NO: 69 - protein, 5 aa, Artificial Sequence
  Antibody clone 1E05 CDRH1
TYAMN

SEQ ID NO: 70 - protein, 17 aa, Artificial Sequence
  Antibody clone 1E05 CDRH2
VISYDGSNKNYVDSVKG

SEQ ID NO: 71 - protein, 15 aa, Artificial Sequence
  Antibody clone 1E05 CDRH3
NFDNSGYAIPDAFDI

SEQ ID NO: 72 - protein, 14 aa, Artificial Sequence
  Antibody clone 1E05 CDRL1
TGSSSNIGAGYDVH

SEQ ID NO: 73 - protein, 7 aa, Artificial Sequence
  Antibody clone 1E05 CDRL2
DNNSRPS

SEQ ID NO: 74 - protein, 11 aa, Artificial Sequence
  Antibody clone 1E05 CDRL3
AAWDDSLGGPV

SEQ ID NO: 75 - protein, 5 aa, Artificial Sequence
  Antibody clone 2A09 CDRH1
NAWMS

SEQ ID NO: 76 - protein, 17 aa, Artificial Sequence
  Antibody clone 2A09 CDRH2
YISRDADITHYPASVKG

SEQ ID NO: 77 - protein, 12 aa, Artificial Sequence
  Antibody clone 2A09 CDRH3
GFDYAGDDAFDI

SEQ ID NO: 78 - protein, 13 aa, Artificial Sequence
  Antibody clone 2A09 CDRL1
SGSSSNIGSNAVN

SEQ ID NO: 79 - protein, 7 aa, Artificial Sequence
  Antibody clone 2A09 CDRL2
GNSDRPS

SEQ ID NO: 80 - protein, 12 aa, Artificial Sequence
  Antibody clone 2A09 CDRL3
AAWDDSLNGRWV

SEQ ID NO: 81 - protein, 5 aa, Artificial Sequence
  Antibody clone 2B08 CDRH1
DYYMS

SEQ ID NO: 82 - protein, 17 aa, Artificial Sequence
  Antibody clone 2B08 CDRH2
LIGHDGNNKYYLDSLEG

SEQ ID NO: 83 - protein, 10 aa, Artificial Sequence
  Antibody clone 2B08 CDRH3
ATDSGYDLLY

SEQ ID NO: 84 - protein, 13 aa, Artificial Sequence
  Antibody clone 2B08 CDRL1
SGSSSNIGNNAVN

SEQ ID NO: 85 - protein, 7 aa, Artificial Sequence
  Antibody clone 2B08 CDRL2
YDDLLPS

SEQ ID NO: 86 - protein, 11 aa, Artificial Sequence
  Antibody clone 2B08 CDRL3
TTWDDSLSGVV

SEQ ID NO: 87 - protein, 5 aa, Artificial Sequence
  Antibody clone 2E08 CDRH1
DYYMS

SEQ ID NO: 88 - protein, 17 aa, Artificial Sequence
  Antibody clone 2E08 CDRH2
AIGFSDDNTYYADSVKG

SEQ ID NO: 89 - protein, 8 aa, Artificial Sequence
  Antibody clone 2E08 CDRH3
GDGSGWSF

SEQ ID NO: 90 - protein, 13 aa, Artificial Sequence
  Antibody clone 2E08 CDRL1
SGSSSNIGNNAVN

SEQ ID NO: 91 - protein, 7 aa, Artificial Sequence
  Antibody clone 2E08 CDRL2
DNNKRPS

SEQ ID NO: 92 - protein, 11 aa, Artificial Sequence
  Antibody clone 2E08 CDRL3
ATWDDSLRGWV

SEQ ID NO: 93 - protein, 5 aa, Artificial Sequence
  Antibody clone 5C04 CDRH1
NYGMH

SEQ ID NO: 94 - protein, 17 aa, Artificial Sequence
  Antibody clone 5C04 CDRH2
VISYDGSNKYYADSVKG

SEQ ID NO: 95 - protein, 7 aa, Artificial Sequence
  Antibody clone 5C04 CDRH2
WRDAFDI

SEQ ID NO: 96 - protein, 14 aa, Artificial Sequence
  Antibody clone 5C04 CDRL1
TGSSSNIGAGYDVH

SEQ ID NO: 97 - protein, 7 aa, Artificial Sequence
  Antibody clone 5C04 CDRL2
SDNQRPS

SEQ ID NO: 98 - protein, 12 aa, Artificial Sequence
  Antibody clone 5C04 CDRL3
AAWDDSLSGSWV

SEQ ID NO: 99 - protein, 5 aa, Artificial Sequence
  Antibody clone 5C05 CDRH1
TYGMH

SEQ ID NO: 100 - protein, 17 aa, Artificial Sequence
  Antibody clone 5C05 CDRH2
VISYDGSNKYYADSVKG

SEQ ID NO: 101 - protein, 8 aa, Artificial Sequence
  Antibody clone 5C05 CDRH3
ENFDAFDV

SEQ ID NO: 102 - protein, 14 aa, Artificial Sequence
  Antibody clone 5C05 CDRL1
TGSSSNIGAGYDVH

SEQ ID NO: 103 - protein, 7 aa, Artificial Sequence
  Antibody clone 5C05 CDRL2
SNSQRPS

SEQ ID NO: 104 - protein, 12 aa, Artificial Sequence
  Antibody clone 5C05 CDRL3
AAWDDSLNGQVV

SEQ ID NO: 105 - protein, 5 aa, Artificial Sequence
  Antibody clone 5D07 CDRH1
TYGMH

SEQ ID NO: 106 - protein, 17 aa, Artificial Sequence
  Antibody clone 5D07 CDRH2
VIAYDGSKKDYADSVKG

SEQ ID NO: 107 - protein, 8 aa, Artificial Sequence
  Antibody clone 5D07 CDRH3
EYRDAFDI

SEQ ID NO: 108 - protein, 14 aa, Artificial Sequence
  Antibody clone 5D07 CDRL1
TGSSSNIGAGYDVH

SEQ ID NO: 109 - protein, 7 aa, Artificial Sequence
  Antibody clone 5D07 CDRL2
GNSNRPS

SEQ ID NO: 110 - protein, 11 aa, Artificial Sequence
  Antibody clone 5D07 CDRL3
AAWDDSVSGWM

SEQ ID NO: 111 - protein, 5 aa, Artificial Sequence
  Antibody clone 5E12 CDRH1
SYGMH

SEQ ID NO: 112 - protein, 17 aa, Artificial Sequence
  Antibody clone 5E12 CDRH2
VISYDGINKDYADSMKG

SEQ ID NO: 113 - protein, 8 aa, Artificial Sequence
  Antibody clone 5E12 CDRH3
ERKDAFDI

SEQ ID NO: 114 - protein, 14 aa, Artificial Sequence
  Antibody clone 5E12 CDRL1
TGSSSNIGAGYDVH

SEQ ID NO: 115 - protein, 7 aa, Artificial Sequence
  Antibody clone 5E12 CDRL2
SNNQRPS

SEQ ID NO: 116 - protein, 11 aa, Artificial Sequence
  Antibody clone 5E12 CDRL3
ATWDDSLNGLV

SEQ ID NO: 117 - protein, 5 aa, Artificial Sequence
  Antibody clone 5G08 CDRH1
NYGMH

SEQ ID NO: 118 - protein, 17 aa, Artificial Sequence
  Antibody clone 5G08 CDRH2
VISYDGSNRYYADSVKG

SEQ ID NO: 119 - protein, 8 aa, Artificial Sequence
  Antibody clone 5G08 CDRH3
DRWNGMDV

SEQ ID NO: 120 - protein, 14 aa, Artificial Sequence
  Antibody clone 5G08 CDRL1
SGSSSNIGAGYDVH

SEQ ID NO: 121 - protein, 7 aa, Artificial Sequence
  Antibody clone 5G08 CDRL2
ANNQRPS

SEQ ID NO: 122 - protein, 12 aa, Artificial Sequence
  Antibody clone 5G08 CDRL3
AAWDDSLNGPWV

SEQ ID NO: 123 - protein, 5 aa, Artificial Sequence
  Antibody clone 5H06 CDRH1
SYGMH

SEQ ID NO: 124 - protein, 17 aa, Artificial Sequence
  Antibody clone 5H06 CDRH2
VISYDGSDTAYADSVKG

SEQ ID NO: 125 - protein, 10 aa, Artificial Sequence
  Antibody clone 5H06 CDRH3
DHSVIGAFDI

SEQ ID NO: 126 - protein, 13 aa, Artificial Sequence
  Antibody clone 5H06 CDRL1
SGSSSNIGSNTVN

SEQ ID NO: 127 - protein, 7 aa, Artificial Sequence
  Antibody clone 5H06 CDRL2
DNNKRPS

SEQ ID NO: 128 - protein, 10 aa, Artificial Sequence
  Antibody clone 5H06 CDRL3
SSYAGSNNVV

SEQ ID NO: 129 - protein, 5 aa, Artificial Sequence
  Antibody clone 6A09 CDRH1
SYGMH

SEQ ID NO: 130 - protein, 17 aa, Artificial Sequence
  Antibody clone 6A09 CDRH2
VTSYDGNTKYYANSVKG

SEQ ID NO: 131 - protein, 10 aa, Artificial Sequence
  Antibody clone 6A09 CDRH3
EDCGGDCFDY

SEQ ID NO: 132 - protein, 14 aa, Artificial Sequence
  Antibody clone 6A09 CDRL1
TGSSSNIGAGYDVH

SEQ ID NO: 133 - protein, 7 aa, Artificial Sequence
  Antibody clone 6A09 CDRL2
GNSNRPS

SEQ ID NO: 134 - protein, 11 aa, Artificial Sequence
  Antibody clone 6A09 CDRL3
AAWDDSLNEGV

SEQ ID NO: 135 - protein, 5 aa, Artificial Sequence
  Antibody clone 6B01 CDRH1
NYGMH

SEQ ID NO: 136 - protein, 17 aa, Artificial Sequence
  Antibody clone 6B01 CDRH2
VISYDGSNKYYADSVKG

SEQ ID NO: 137 - protein, 9 aa, Artificial Sequence
  Antibody clone 6B01 CDRH3
DQLGEAFDI

SEQ ID NO: 138 - protein, 14 aa, Artificial Sequence
  Antibody clone 6B01 CDRL1
TGSSSNIGAGYDVH

SEQ ID NO: 139 - protein, 7 aa, Artificial Sequence
  Antibody clone 6B01 CDRL2
DNNKRPS

SEQ ID NO: 140 - protein, 11 aa, Artificial Sequence
  Antibody clone 6B01 CDRL3
ATWDDSLSGPV

SEQ ID NO: 141 - protein, 5 aa, Artificial Sequence
  Antibody clone 6C11 CDRH1
DYGMS

SEQ ID NO: 142 - protein, 17 aa, Artificial Sequence
  Antibody clone 6C11 CDRH2
AISGSGSSTYYADSVKG

SEQ ID NO: 143 - protein, 8 aa, Artificial Sequence
  Antibody clone 6C11 CDRH3
GDIDYFDY

SEQ ID NO: 144 - protein, 14 aa, Artificial Sequence
  Antibody clone 6C11 CDRL1
TGSSSNFGAGYDVH

SEQ ID NO: 145 - protein, 7 aa, Artificial Sequence
  Antibody clone 6C11 CDRL3
ENNKRPS

SEQ ID NO: 146 - protein, 11 aa, Artificial Sequence
  Antibody clone 6C11 CDRL3
AAWDDSLNGPV

SEQ ID NO: 147 - protein, 5 aa, Artificial Sequence
  Antibody clone 6C12 CDRH1
SYGMH

SEQ ID NO: 148 - protein, 17 aa, Artificial Sequence
  Antibody clone 6C12 CDRH2
VISYDGSNKYYADSVKG

SEQ ID NO: 149 - protein, 8 aa, Artificial Sequence
  Antibody clone 6C12 CDRH3
ERRDAFDI

SEQ ID NO: 150 - protein, 14 aa, Artificial Sequence
  Antibody clone 6C12 CDRL1
TGSSSNIGAGYDVH

SEQ ID NO: 151 - protein, 7 aa, Artificial Sequence
  Antibody clone 6C12 CDRL2
SDNQRPS

SEQ ID NO: 152 - protein, 9 aa, Artificial Sequence
  Antibody clone 6C12 CDRL3
ATWDSDTPV

SEQ ID NO: 153 - protein, 5 aa, Artificial Sequence
  Antibody clone 6D01 CDRH1
SYGMH

SEQ ID NO: 154 - protein, 17 aa, Artificial Sequence
  Antibody clone 6D01 CDRH2
VISYDGSNKYYADSVKG

SEQ ID NO: 155 - protein, 10 aa, Artificial Sequence
  Antibody clone 6D01 CDRH3
DHSAAGYFDY

SEQ ID NO: 156 - protein, 13 aa, Artificial Sequence
  Antibody clone 6D01 CDRL1
SGSSSNIGSNTVN

SEQ ID NO: 157 - protein, 7 aa, Artificial Sequence
  Antibody clone 6D01 CDRL2
GNSIRPS

SEQ ID NO: 158 - protein, 11 aa, Artificial Sequence
  Antibody clone 6D01 CDRL3
ASWDDSLSSPV

SEQ ID NO: 159 - protein, 5 aa, Artificial Sequence
  Antibody clone 6G03 CDRH1
SYGMH

SEQ ID NO: 160 - protein, 17 aa, Artificial Sequence
  Antibody clone 6G03 CDRH2
GISWDSAIIDYAGSVKG

SEQ ID NO: 161 - protein, 10 aa, Artificial Sequence
  Antibody clone 6G03 CDRH3
DEAAAGAFDI

SEQ ID NO: 162 - protein, 14 aa, Artificial Sequence
  Antibody clone 6G03 CDRL1
TGSSSNIGAGYDVH

SEQ ID NO: 163 - protein, 7 aa, Artificial Sequence
  Antibody clone 6G03 CDRL2
GNTDRPS

SEQ ID NO: 164 - protein, 12 aa, Artificial Sequence
  Antibody clone 6G03 CDRL3
AAWDDSLSGPVV

SEQ ID NO: 165 - protein, 5 aa, Artificial Sequence
  Antibody clone 6G08 CDRH1
SYGIS

SEQ ID NO: 166 - protein, 17 aa, Artificial Sequence
  Antibody clone 6G08 CDRH2
GISGSGGNTYYADSVKG

SEQ ID NO: 167 - protein, 12 aa, Artificial Sequence
  Antibody clone 6G08 CDRH3
SVGAYANDAFDI

SEQ ID NO: 168 - protein, 14 aa, Artificial Sequence
  Antibody clone 6G08 CDRL1
TGSSSNIGAGYDVH

SEQ ID NO: 169 - protein, 7 aa, Artificial Sequence
  Antibody clone 6G08 CDRL2
GDTNRPS

SEQ ID NO: 170 - protein, 11 aa, Artificial Sequence
  Antibody clone 6G08 CDRL3
AAWDDSLNGPV

SEQ ID NO: 171 - protein, 5 aa, Artificial Sequence
  Antibody clone 6G11 CDRH1
SYGMH

SEQ ID NO: 172 - protein, 17 aa, Artificial Sequence
  Antibody clone 6G11 CDRH2
VISYDGSNKYYADSVKG

SEQ ID NO: 173 - protein, 8 aa, Artificial Sequence
  Antibody clone 6G11 CDRH3
ELYDAFDI

SEQ ID NO: 174 - protein, 14 aa, Artificial Sequence
  Antibody clone 6G11 CDRL1
TGSSSNIGAGYDVH

SEQ ID NO: 175 - protein, 7 aa, Artificial Sequence
  Antibody clone 6G11 CDRL2
ADDHRPS

SEQ ID NO: 176 - protein, 11 aa, Artificial Sequence
  Antibody clone 6G11 CDRL3
ASWDDSQRAVI

SEQ ID NO: 177 - protein, 5 aa, Artificial Sequence
  Antibody clone 6H08 CDRH1
NYGMH

SEQ ID NO: 178 - protein, 17 aa, Artificial Sequence
  Antibody clone 6H08 CDRH2
VISYDGSNKYYADSVKG

SEQ ID NO: 179 - protein, 8 aa, Artificial Sequence
  Antibody clone 6H08 CDRH3
EYKDAFDI

SEQ ID NO: 180 - protein, 13 aa, Artificial Sequence
  Antibody clone 6H08 CDRL1
TGSSSNIGSNTVN

SEQ ID NO: 181 - protein, 7 aa, Artificial Sequence
  Antibody clone 6H08 CDRL2
DNNKRPS

SEQ ID NO: 182 - protein, 9 aa, Artificial Sequence
  Antibody clone 6H08 CDRL3
QAWGTGIRV

SEQ ID NO: 183 - protein, 5 aa, Artificial Sequence
  Antibody clone 7C07 CDRH1
SYGMH

SEQ ID NO: 184 - protein, 17 aa, Artificial Sequence
  Antibody clone 7C07 CDRH2
VISYDGSNKYYADSVKG

SEQ ID NO: 185 - protein, 9 aa, Artificial Sequence
  Antibody clone 7C07 CDRH3
EFGYIILDY

SEQ ID NO: 186 - protein, 13 aa, Artificial Sequence
  Antibody clone 7C07 CDRL1
SGSSSNIGSNTVN

SEQ ID NO: 187 - protein, 7 aa, Artificial Sequence
  Antibody clone 7C07 CDRL2
RDYERPS

SEQ ID NO: 188 - protein, 11 aa, Artificial Sequence
  Antibody clone 7C07 CDRL1
MAWDDSLSGVV

SEQ ID NO: 189 - protein, 5 aa, Artificial Sequence
  Antibody clone 4B02 CDRH1
NHGMH

SEQ ID NO: 190 - protein, 17 aa, Artificial Sequence
  Antibody clone 4B02 CDRH2
VISYDGTNKYYADSVRG

SEQ ID NO: 191 - protein, 8 aa, Artificial Sequence
  Antibody clone 4B02 CDRH3
ETWDAFDV

SEQ ID NO: 192 - protein, 13 aa, Artificial Sequence
  Antibody clone 4B02 CDRL1
SGSSSNIGSNNAN

SEQ ID NO: 193 - protein, 7 aa, Artificial Sequence
  Antibody clone 4B02 CDRL2
DNNKRPS

SEQ ID NO: 194 - protein, 9 aa, Artificial Sequence
  Antibody clone 4B02 CDRL3
QAWDSSTVV

SEQ ID NO: 195 - protein, 330 aa, Homo sapiens
  IgG constant region heavy (CH)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 196 - protein, 330 aa, Mus musculus
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYASTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 197 - protein, 105 aa, Mus musculus
QPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

(19) United States
(12) Patent Application Publication
(10) Pub. No.: US 2024/0092912 A1
(43) Pub. Date: Mar. 21, 2024

(54) NOVEL COMBINATIONS OF ANTIBODIES AND USES THEREOF

(71) Applicants: BIOINVENT INTERNATIONAL AB, Lund (SE); UNIVERSITY OF SOUTHAMPTON, Southhampton, Hampshire (GB)

(72) Inventors: Björn FRENDÉUS, Lund (SE); Linda MÅRTENSSON, Bjärred (SE); Ingrid TEIGE, Lund (SE); Mark CRAGG, Southampton (GB); Robert OLDHAM, Southampton (GB); Stephen BEERS, Southampton (GB); Ali ROGHANIAN, Southampton (GB)

(21) Appl. No.: 18/281,530

(22) PCT Filed: Mar. 9, 2022
(86) PCT No.: PCT/EP2022/056037

§ 371 Date: Sep. 11, 2023

(30) Foreign Application Priority Data
Mar. 9, 2021 (EP) 21161460.7

(51) Int. Cl. C07K 16/28; A61P 35/00

(52) U.S. Cl. CPC C07K 16/283; A61P 35/00; C07K 16/2818; C07K 16/2827; A61K 2039/507

(57) ABSTRACT

The present invention generally relates to antibody combinations and uses thereof.

### FIELD OF THE DISCLOSURE

The present disclosure generally relates to antibody combinations and uses thereof.

### Background

Immunotherapy with therapeutic antibodies has increased survival for patients with hematologic and solid cancers. The clinically successful antibodies exert anti-tumour activity by targeting tumour cells directly [1-4], or by targeting and activating immune cells that seek and kill cancer cells in the tumour microenvironment (so called “immune checkpoint antibodies”) [5-13]. While both types of antibody are highly potent with cancer curative potential, a significant proportion of patients fail to respond, or acquire resistance during the course of therapy [14-17].

It has long been known that there is a critical role for FcγRs in controlling therapeutic activity of tumour-targeting antibodies. However, the role of FcγRs controlling efficacy and resistance of immune modulatory antibodies, e.g. those targeting the immune inhibitory checkpoints CTLA-4 and PD-1/PD-L1, is less predictable. Antibodies targeting CTLA-4, PD-1 and PD-L1 were developed based on their ability to block inhibitory signaling in effector T cells, i.e. “unleashing the brakes” of the immune system to eradicate cancer cells, which themselves do not typically express CTLA-4, PD-1/PD-L1 or FcγRs.

Surprisingly therefore, the inventors recently reported a role for FcγRs underlying the therapeutic activity of the CTLA-4 specific antibody ipilimumab [18]. Melanoma patients expressing high affinity single nucleotide polymorphisms (SNPs) of activating FcγRIIIA showed improved survival in response to ipilimumab therapy, despite melanoma tumours being negative for CTLA-4. Conversely, independent studies of antibodies directed to PD-1 have indicated a detrimental role for FcγRs in their anti-tumour activity, with conflicting proposed underlying mechanisms and roles of activating and inhibitory FcγRs [19, 20]. The effect of FcγR-blockade on the therapeutic effect of anti-CTLA-4 and anti-PD-1/PD-L1 antibodies is therefore unpredictable.

Against that background, the inventors have assessed the effect of FcγR-blockade using FcγR specific antibodies on the therapeutic activity of anti-CTLA-4 and PD-1 antibodies in vivo. Surprisingly, it has been found that FcγR-blockade using antibodies engineered for silenced Fc:FcγR-engagement enhances the therapeutic activity of anti-CTLA-4 and anti-PD1/PD-L1 antibodies when used in combination. This has implications for treatment of patients that are resistant to treatment with anti-CTLA-4 and anti-PD1/PD-L1 antibodies. Furthermore, the inventors have also found that FcγR-blockade using antibodies engineered for silenced Fc:FcγR-engagement unexpectedly allows a lower therapeutic dose of an anti-CTLA-4 antibody to be used, thereby reducing the possibility of unwanted side effects and toxicity.

### SUMMARY

As discussed above, the disclosure generally relates to a combination comprising a first antibody molecule, a second antibody molecule and a third antibody molecule. The first to seventh aspects of the disclosure relating to this are discussed below: In a first aspect, the disclosure provides a combination comprising a first antibody molecule that specifically binds to FcγRIIb via its Fab region, and that lacks an Fc region or has reduced binding to Fcγ receptors via its Fc region, a second antibody molecule that specifically binds to PD-1 or PD-L1, and a third antibody molecule that specifically binds to CTLA-4 and that binds to at least one Fcγ receptor via its Fc region, for use in treating cancer in a patient, wherein the cancer is resistant to treatment with an antibody molecule that specifically binds to PD-1 or PD-L1, and/or an antibody molecule that specifically binds to CTLA-4.

In a second aspect, the disclosure provides the use of a first antibody molecule that specifically binds to FcγRIIb via its Fab region, and that lacks an Fc region or has reduced binding to Fcγ receptors via its Fc region, a second antibody molecule that specifically binds to PD-1 or PD-L1, and a third antibody molecule that specifically binds to CTLA-4 and that binds to at least one Fcγ receptor via its Fc region, in the manufacture of a medicament for treating cancer in a patient, wherein the cancer is resistant to treatment with an antibody molecule that specifically binds to PD-1 or PD-L1, and/or an antibody molecule that specifically binds to CTLA-4.

In a third aspect, the disclosure provides a method for treating cancer in a patient, the method comprising administering to the patient (i) a first antibody molecule that specifically binds to FcγRIIb via its Fab region, and that lacks an Fc region or has reduced binding to Fcγ receptors via its Fc region, (ii) a second antibody molecule that specifically binds to PD-1 or PD-L1, and (iii) a third antibody molecule that specifically binds to CTLA-4 and that binds to at least one Fcγ receptor via its Fc region, wherein the cancer is resistant to treatment with an antibody molecule that specifically binds to PD-1 or PD-L1, and/or an antibody molecule that specifically binds to CTLA-4.

In a fourth aspect, the disclosure provides a first antibody molecule that specifically binds to FcγRIIb via its Fab region, and that lacks an Fc region or has reduced binding to Fcγ receptors via its Fc region, for use in combination with (i) a second antibody molecule that specifically binds to PD-1 or PD-L1, and (ii) a third antibody molecule that specifically binds to CTLA-4 and that binds to at least one Fcγ receptor via its Fc region, for treating cancer in a patient, wherein the cancer is resistant to treatment with an antibody molecule that specifically binds to PD-1 or PD-L1, and/or an antibody molecule that specifically binds to CTLA-4.

In a fifth aspect, the disclosure provides a first antibody molecule that specifically binds to FcγRIIb via its Fab region, and that lacks an Fc region or has reduced binding to Fcγ receptors via its Fc region, for use in treating cancer in a patient; characterised in that the first antibody molecule reduces and/or prevents resistance in the cancer to treatment with an antibody molecule that specifically binds to PD-1 or PD-L1, and/or an antibody molecule that specifically binds to CTLA-4.

In a sixth aspect, the disclosure provides a pharmaceutical composition comprising a first antibody molecule that specifically binds to FcγRIIb via its Fab region, and that lacks an Fc region or has reduced binding to Fcγ receptors via its Fc region, a second antibody molecule that specifically binds to PD-1 or PD-L1, and a third antibody molecule that specifically binds to CTLA-4 and that binds to at least one Fcγ receptor via its Fc region.

In a seventh aspect, the disclosure provides a kit comprising a first antibody molecule that specifically binds to FcγRIIb via its Fab region, and that lacks an Fc region or has reduced binding to Fcγ receptors via its Fc region, a second antibody molecule that specifically binds to PD-1 or PD-L1, and a third antibody molecule that specifically binds to CTLA-4 and that binds to at least one Fcγ receptor via its Fc region.

The first antibody molecule described herein specifically binds to FcγRIIb via its Fab region, and lacks an Fc region or has reduced binding to Fcγ receptors via its Fc region. Fc receptors are well known in the art as membrane proteins which are found on the cell surface of immune effector cells, such as macrophages. The name is derived from their binding specificity for the Fc region of antibodies, which is the usual way an antibody binds to the receptor. However, certain antibodies can also bind the Fc receptors via the antibodies' CDR sequences in the case of antibodies specifically binding to one or more Fc receptors.

A subgroup of the Fc receptors are Fcγ receptors (Fc-gamma receptors, FcgammaR), which are specific for IgG antibodies. There are two types of Fcγ receptors: activating Fcγ receptors (also denoted activatory Fcγ receptors) and inhibitory Fcγ receptors. The activating and the inhibitory receptors transmit their signals via immunoreceptor tyrosine-based activation motifs (ITAM) or immunoreceptor tyrosine-based inhibitory motifs (ITIM), respectively. In humans, FcγRIIb (CD32b) is an inhibitory Fcγ receptor, while FcγRI (CD64), FcγRIIa (CD32a), FcγRIIc (CD32c), FcγRIIIa (CD16a) and FcγRIV are activating Fcγ receptors. FcγγRIIIb is a GPI-linked receptor expressed on neutrophils that lacks an ITAM motif but through its ability to cross-link lipid rafts and engage with other receptors is also considered activatory. In mice, the activating receptors are FcγRI, FcγRIII and FcγRIV.

It is well-known that antibodies modulate immune cell activity through interaction with Fcγ receptors. Specifically, how antibody immune complexes modulate immune cell activation is determined by their relative engagement of activating and inhibitory Fcγ receptors. Different antibody isotypes bind with different affinity to activating and inhibitory Fcγ receptors, resulting in different A:I ratios (activation:inhibition ratios) (Nimmerjahn et al; Science. 2005 Dec. 2; 310(5753):1510-2).

By binding to an inhibitory Fcγ receptor, an antibody can inhibit, block and/or downmodulate effector cell functions.

By binding to an activatory Fcγ receptor, an antibody can activate effector cell functions and thereby trigger mechanisms such as antibody-dependent cellular cytotoxicity (ADCC), antibody dependent cellular phagocytosis (ADCP), cytokine release, and/or antibody dependent endocytosis, as well as NETosis (i.e. activation and release of NETs, Neutrophil extracellular traps) in the case of neutrophils. Antibody binding to an activating Fcγ receptor can also lead to an increase in certain activation markers, such as CD40, MHCII, CD38, CD80 and/or CD86.

The first antibody molecule according to the disclosure that specifically binds FcγRIIb, binds to or interacts with this Fcγ receptor via the Fab region of the antibody, i.e. via the antigen-binding region on an antibody that binds to antigens which is composed of one constant and one variable domain of each of the heavy and the light chain. In particular, it binds to FcγRIIb present on an immune effector cell, and in particular to FcγRIIb present on the surface of an immune effector cell. If this antibody would have had a usual or ordinary Fc region, the antibody could also have bound to an activating Fcγ receptor through normal interaction between the Fc region and Fc receptor. However, according to the disclosure, the antibody molecule that specifically binds FcγRIIb completely lacks Fc region or has reduced binding to Fcγ receptors, which means that the antibody molecule that specifically binds or interacts with FcγRIIb via the Fab region binds poorly to or cannot at all bind to or interact with Fcγ receptors.

This appears to have at least two therapeutically important consequences: First, the lack of Fc-mediated binding to activating FcγRs leaves a greater number of activating Fc gamma receptors available for binding to Fcs of (other) therapeutic anti-cancer antibodies, for instance the second and/or third antibody molecules as defined herein. This is important since clustering of an increasing number of activating FcγRs (vs inhibitory FcγRs; Nimmerjahn et al; Science. 2005 Dec. 2; 310(5753):1510-2) is known to increase effector cell mediated target cell deletion, a mechanism underlying activity of both checkpoint inhibitor, immune agonist, and other immunomodulatory antibodies, such as anti-IL-2R.

Second, the lack of, or reduced, Fc-mediated binding to inhibitory FcγR was shown to reduce inhibitory signalling in FcγR-expressing immune effector cells. Thus, lack of or reduced Fc-mediated binding to FcγR of the FcγRIIB targeting antibody likely improves therapeutic efficacy by at least two mechanisms, involving both improved activatory FcγR and reduced inhibitory Fcγ signalling in immune effector cells in response to a second immunomodulatory anti-cancer antibody.

In the case of the above first to seventh aspects of the present disclosure, this is advantageous as it allows the antibodies that specifically bind CTLA-4 (and/or PD-1/PD-L1, in some embodiments) to both bind their target molecules on immune effector cells, which upregulates the immune response to cancer cells, and also allows these antibodies to bind specifically to activating FcγRs, further upregulating the immune response. This effect can surprisingly restore the therapeutic effect of antibodies that specifically bind CTLA-4/PD-1/PD-L1 in patients who are resistant to such therapies.

By “lacks an Fc region” we include any antibody or antibody fragment thereof that has no Fc region, which therefore prevents Fc mediated binding of the antibody or antibody fragment to Fcγ receptors. Such antibodies retain specific binding to the FcγRIIb via the Fab region. Examples of antibody fragments that lack an Fc region and that are compatible with this embodiment of the disclosure include, but are not limited to: Fab, Fab′, F(ab)2, Fv, scFv, dsFv, VH, VL, or PEGYLATED versions thereof.

By “reduced binding to Fcγ receptors” (also referred to as “binding with reduced affinity”) we include that the antibody molecule has reduced Fc mediated binding to Fcγ receptors, or in other words that the Fc region of the antibody molecule that specifically binds FcγRIIb binds to an activating Fcγ receptor with lower affinity than the Fc region of a normal human IgG1. The reduction in binding can be assessed using techniques such as surface plasmon resonance. In this context “normal IgG1” means a conventionally produced IgG1 with a non-mutated Fc region that has not been produced so as to alter its glycosylation. As a reference for this “normal IgG1” it is possible to use rituximab produced in CHO cells without any modifications (Tipton et al, Blood 2015 125:1901-1909; rituximab is described in, for example, EP 0 605 442). Human IgG2 and human IgG4 are examples of antibody isotypes that bind with reduced affinity to Fcγ receptors compared with human IgG1. Therefore, antibodies based on human IgG2 and IgG4 have “reduced binding to Fcγ receptors” within the meaning of this term.

“Reduced binding” may mean that binding of the Fc region of the antibody molecule that specifically binds FcγRIIb binds to an activating Fcγ receptor is at least 10 fold reduced for all Fc receptors compared to the binding of the Fc region of a normal human IgG1 to the same receptors. In some embodiments it is at least 20 fold reduced. In some embodiments it is at least 30 fold reduced. In some embodiments it is at least 40 fold reduced. In some embodiments it is at least 50 fold reduced. In some embodiments it is at least 60 fold reduced. In some embodiments it is at least 70 fold reduced.

In some embodiments, the antibody molecule that specifically binds FcγRIIb may be a llama antibody, and in particular a llama hcIgG. Like all mammals, camelids produce conventional antibodies made of two heavy chains and two light chains bound together with disulphide bonds in a Y shape (IgG₁). However, they also produce two unique subclasses of immunoglobulin G, IgG₂ and IgG₃, also known as heavy chain IgG (hcIgG). These antibodies are made of only two heavy chains that lack the CH1 region but still bear an antigen binding domain at their N-terminus called V_{H}H. Conventional Ig requires the association of variable regions from both heavy and light chains to allow a high diversity of antigen-antibody interactions. Although isolated heavy and light chains still show this capacity, they exhibit very low affinity⁴ when compared to paired heavy and light chains. The unique feature of hcIgG is the capacity of their monomeric antigen binding regions to bind antigens with specificity, affinity and especially diversity that are comparable to conventional antibodies without the need of pairing with another region.

In some embodiments reduced binding means that the antibody has a 20 fold reduced affinity with regards to binding to FcγRI.

In order to obtain reduced binding of an IgG antibody, such as an IgG1 or IgG2 antibody, to an Fc receptor, it is possible to modify the Fc region of the IgG antibody by aglycosylation. Such aglycosylation, for example of an IgG1 antibody, may for example be achieved by an amino acid substitution of the asparagine in position 297 (N297X) in the antibody chain. The substation may be with a glutamine (N297Q), or with an alanine (N297A), or with a glycine (N297G), or with an asparagine (N297D), or by a serine (N297S). In some preferred embodiments, the substitution is with a glutamine (N297Q).

The Fc region may be modified by further substitutions, for example as described by Jacobsen F W et al., JBC 2017, 292, 1865-1875, (see e.g. Table 1). Such additional substitutions include L242C, V259C, A287C, R292C, V302C, L306C, V323C, I332C, and/or K334C. Such modifications also include the following combinations of substitutions in an IgG1:

### L242C, N297G, K334C,

### A287C, N297G, L306C,

### R292C, N297G, V302C,

### N297G, V323C, 1332C, and

### V259C, N297G, L306C.

Alternatively, the carbohydrate in the Fc region can be cleaved enzymatically and/or the cells used for producing the antibody can be grown in media that impairs carbohydrate addition and/or cells engineered to lack the ability to add the sugars can be used for the antibody production, or by production of antibodies in host cells that do not glycosylate or do not functionally glycosylate antibodies e.g. prokaryotes including E. coli, as explained above.

Reduced affinity for Fc gamma receptors can further be achieved through engineering of amino acids in the antibody Fc region (such modifications have previously been described by e.g. Xencor, Macrogenics, and Genentech), or by production of antibodies in host cells that do not glycosylate or does not functionally glycosylate antibodies e.g. prokaryotes including E. coli.

In addition to having reduced binding to Fcγ receptors through the Fc region, it is in some embodiments preferred that the antibody molecule that specifically binds FcγRIIb does not give rise to phosphorylation of FcγRIIb when binding the target. Phosphorylation of the ITIM of FcγRIIb is an inhibitory event that blocks the activity in the immune cell.

Fc gamma receptor expressing immune effector cell refers herein to principally innate effector cells, and includes specifically macrophages, neutrophils, monocytes, natural killer (NK) cells, basophils, eosinophils, mast cells, and platelets. Cytotoxic T cells and memory T cells do not typically express FcγRs, but may do so in specific circumstances. In some embodiments the immune effector cell is an innate immune effector cell. In some embodiments, the immune effector cell is a macrophage.

In some embodiments the antibody molecule that specifically binds FcγRIIb is a human antibody.

In some embodiments, the antibody molecule that specifically binds FcγRIIb is an antibody of human origin, i.e. an originally human antibody that has been modified as described herein.

In some embodiments, the antibody molecule that specifically binds FcγRIIb is a humanized antibody, i.e. an originally non-human antibody that has been modified to increase its similarity to a human antibody. The humanized antibodies may, for example, be murine antibodies or llama antibodies.

As discussed above, the first antibody may be a monoclonal antibody or an antibody molecule of monoclonal origin.

In some embodiments, the antibody molecule that specifically binds FcγRIIb comprises the following constant regions (CH and CL):

IgG1-CH
[SEQ ID NO: 1]
ASTKGPSVFPLAPSSKSTSGGTAALGCLVKDYFPEPVTVS
WNSGALTSGVHTFPAVLQSSGLYSLSSVVTVPSSSLGTQT
YICNVNHKPSNTKVDKKVEPKSCDKTHTCPPCPAPELLGG
PSVFLFPPKPKDTLMISRTPEVTCVVVDVSHEDPEVKFNW
YVDGVEVHNAKTKPREEQYNSTYRVVSVLTVLHQDWLNGK
EYKCKVSNKALPAPIEKTISKAKGQPREPQVYTLPPSRDE
LTKNQVSLTCLVKGFYPSDIAVEWESNGPENNYKTTPP-V
LDSDGSFFLYSKLTVDKSRWQQGNVFSCSVMHEALHNHYT
QKSLSLSPGK
IgG1-CL
[SEQ ID NO: 2]
QPKAAPSVTLFPPSSEELQANKATLVCLISDFYPGAVTVA
WKADSSPVKAGVETTTPSKQSNNKYAASSYLSLTPEQWKS
HRSYSCQVTHEGSTVEKTVAPTECS

These constant regions (SEQ ID NO: 1 and SEQ ID NO: 2) are of human origin. The Fc region is further modified for reduced binding to Fcγ receptors via its Fc region. As mentioned herein, it is in some embodiments preferred that SEQ ID NO: 1 has been aglycosylated through an N297Q substitution, and the IgG1-CH has then the following CH sequence [SEQ ID NO: 195], with the 297 Q residue is marked in bold:

ASTKGPSVFPLAPSSKSTSGGTAALGCLVKDYFPEPVTVS
WNSGALTSGVHTFPAVLQSSGLYSLSSVVTVPSSSLGTQT
YICNVNHKPSNTKVDKKVEPKSCDKTHTCPPCPAPELLGG
PSVFLFPPKPKDTLMISRTPEVTCVVVDVSHEDPEVKFNW
YVDGVEVHNAKTKPREEQYQSTYRVVSVLTVLHQDWLNGK
EYKCKVSNKALPAPIEKTISKAKGQPREPQVYTLPPSRDE
LTKNQVSLTCLVKGFYPSDIAVEWESNGQPENNYKTTPPV
LDSDGSFFLYSKLTVDKSRWQQGNVFSCSVMHEALHNHYT
QKSLSLSPGK

In some embodiments and/or examples, murine antibody molecules are used. These may also be used for surrogate antibodies. These may then comprise the following constant regions (CH and CL):

CH
[SEQ ID NO: 196]
AKTTAPSVYPLAPVCGDTTGSSVTLGCLVKGYFPEPVTLT
WNSGSLSSGVHTFPAVLQSDLYTLSSSVTVTSSTWPSQSI
TCNVAHPASSTKVDKKIEPRGPTIKPCPPCKCPAPNLLGG
PSVFIFPPKIKDVLMISLSPIVTCVVVDVSEDDPDVQISW
FVNNVEVHTAQTQTHREDYASTLRVVSALPIQHQDWMSGK
EFKCKVNNKDLPAPIERTISKPKGSVRAPQVYVLPPPEEE
MTKKQVTLTCMVTDFMPEDIYVEWTNNGKTELNYKNTEPV
LDSDGSYFMYSKLRVEKKNWVERNSYSCSVVHEGLHNHHT
TKSFSRTPGK
CL
[SEQ ID NO: 197]
QPKSSPSVTLFPPSSEELETNKATLVCTITDFYPGVVTVD
WKVDGTPVTQGMETTQPSKQSNNKYMASSYLTLTARAWER
HSSYSCQVTHEGHTVEKSLSRADCS

These constant regions (SEQ ID NO: 196 and SEQ ID NO: 197) are thus of murine origin. SEQ ID NO: 196 comprises the N297A mutation (the 297 A residue is marked in bold in the sequence above). This N297A mutation in the murine sequence corresponds to the N297Q mutation in the human sequence.

In some embodiments, the antibody molecule that specifically binds FcγRIIb comprises one or more sequences of the following clones:

Antibody clone: 1A01
1A01-VH
[SEQ ID NO: 3]
EVQLLESGGGLVQPGGSLRLSCAASGFTFSDYYMNWIRQT
PGKGLEWVSLIGWDGGSTYYADSVKGRFTISRDNSENTLY
LQMNSLRAEDTAVYYCARAYSGYELDYWGQGTLVTVSS
1A01-VL
[SEQ ID NO: 27]
QSVLTQPPSASGTPGQRVTISCSGSSSNIGNNAVNWYQQL
PGTAPKLLIYDNNNRPSGVPDRFSGSKSGTSASLAISGLR
SEDEADYYCAAWDDSLNASIFGGGTKLTVLG
CDR regions
CDRH1:
[SEQ ID NO: 51]
DYYMN
CDRH2:
[SEQ ID NO: 52]
LIGWDGGSTYYADSVKG
CDRH3:
[SEQ ID NO: 53]
AYSGYELDY
CDRL1:
[SEQ ID NO: 54]
SGSSSNIGNNAVN
CDRL2:
[SEQ ID NO: 55]
DNNNRPS
CDRL3:
[SEQ ID NO: 56]
AAWDDSLNASI
Antibody clone: 1B07
1B07-VH
[SEQ ID NO: 4]
EVQLLESGGGLVQPGGSLRLSCAASGFTFSSYGMHWVRQA
PGKGLEWVAFTRYDGSNKYYADSVRGRFTISRDNSKNTLY
LQMNSLRAEDTAVYYCARENIDAFDVWGQGTLVTVSS
1B07-VL
[SEQ ID NO: 28]
QSVLTQPPSASGTPGQRVTISCSGSSSNIGNNAVNWYQQL
PGTAPKLLIYDNQQRPSGVPDRFSGSKSGTSASLAISGLR
SEDEADYYCEAWDDRLFGPVFGGGTKLTVLG
CDR regions
CDRH1:
[SEQ ID NO: 57]
SYGMH
CDRH2:
[SEQ ID NO: 58]
FTRYDGSNKYYADSVRG
CDRH3:
[SEQ ID NO: 59]
ENIDAFDV
CDRL1:
[SEQ ID NO: 60]
SGSSSNIGNNAVN
CDRL2:
[SEQ ID NO: 61]
DNQQRPS
CDRL3:
[SEQ ID NO: 62]
WDDRLFGPV
Antibody clone: 1C04
1C04-VH
[SEQ ID NO: 5]
EVQLLESGGGLVQPGGSLRLSCAASGFTFSSYAMSWVRQA
PGKGLEWVSSISDSGAGRYYADSVEGRFTISRDNSKNTLY
LQMNSLRAEDTAVYYCARTHDSGELLDAFDIWGQGTLVTV
SS
1C04-VL
[SEQ ID NO: 29]
QSVLTQPPSASGTPGQRVTISCSGSSSNIGSNHVLWYQQL
PGTAPKLLIYGNSNRPSGVPDRFSGSKSGTSASLAISGLR
SEDEADYYCAAWDDSLNGWVFGGGTKLTVLG
CDR regions
CDRH1:
[SEQ ID NO: 63]
SYAMS
CDRH2:
[SEQ ID NO: 64]
SISDSGAGRYYADSVEG
CDRH3:
[SEQ ID NO: 65]
THDSGELLDAFDI
CDRL1:
[SEQ ID NO: 66]
SGSSSNIGSNHVL
CDRL2:
[SEQ ID NO: 67]
GNSNRPS
CDRL3:
[SEQ ID NO: 68]
AAWDDSLNGWV
Antibody clone: 1E05
1E05-VH
[SEQ ID NO: 6]
EVQLLESGGGLVQPGGSLRLSCAASGFTFSTYAMNWVRQV
PGKGLEWVAVISYDGSNKNYVDSVKGRFTISRDNSKNTLY
LQMNSLRAEDTAVYYCARNEDNSGYAIPDAFDIWGQGTLV
TVSS
1E05-VL
[SEQ ID NO: 30]
QSVLTQPPSASGTPGQRVTISCTGSSSNIGAGYDVHWYQQ
LPGTAPKLLIYDNNSRPSGVPDRFSGSKSGTSASLAISGL
RSEDEADYYCAAWDDSLGGPVFGGGTKLTVLG
CDR regions
CDRH1:
[SEQ ID NO: 69]
TYAMN
CDRH2:
[SEQ ID NO: 70]
VISYDGSNKNYVDSVKG
CDRH3:
[SEQ ID NO: 71]
NFDNSGYAIPDAFDI
CDRL1:
[SEQ ID NO: 72]
TGSSSNIGAGYDVH
CDRL2:
[SEQ ID NO: 73]
DNNSRPS
CDRL3:
[SEQ ID NO: 74]
AAWDDSLGGPV
Antibody clone: 2A09
2A09-VH
[SEQ ID NO: 7]
EVQLLESGGGLVQPGGSLRLSCAASGFTFSNAWMSWVRQA
PGKGLEWVAYISRDADITHYPASVKGRFTISRDNSKNTLY
LQMNSLRAEDTAVYYCTTGFDYAGDDAFDIWGQGTLVTVS
S
2A09-VL
[SEQ ID NO: 31]
QSVLTQPPSASGTPGQRVTISCSGSSSNIGSNAVNWYQQL
PGTAPKLLIYGNSDRPSGVPDRFSGSKSGTSASLAISGLR
SEDEADYYCAAWDDSLNGRWVFGGGTKLTVLG
CDR regions
CDRH1:
[SEQ ID NO: 75]
NAWMS
CDRH2:
[SEQ ID NO: 76]
YISRDADITHYPASVKG
CDRH3:
[SEQ ID NO: 77]
GFDYAGDDAFDI
CDRL1:
[SEQ ID NO: 78]
SGSSSNIGSNAVN
CDRL2:
GNSDRPS
[SEQ ID NO: 79]
CDRL3:
[SEQ ID NO: 80]
AAWDDSLNGRWV
Antibody clone: 2B08
2B08-VH
[SEQ ID NO: 8]
EVQLLESGGGLVQPGGSLRLSCAASGFTFSDYYMSWVRQA
PGKGLEWVALIGHDGNNKYYLDSLEGRFTISRDNSKNTLY
LQMNSLRAEDTAVYYCARATDSGYDLLYWGQGTLVTVSS
2B08-VL
[SEQ ID NO: 32]
QSVLTQPPSASGTPGQRVTISCSGSSSNIGNNAVNWYQQL
PGTAPKLLIYYDDLLPSGVPDRFSGSKSGTSASLAISGLR
SEDEADYYCTTWDDSLSGVVFGGGTKLTVLG
CDR regions
CDRH1:
[SEQ ID NO: 81]
DYYMS
CDRH2:
[SEQ ID NO: 82]
LIGHDGNNKYYLDSLEG
CDRH3:
[SEQ ID NO: 83]
ATDSGYDLLY
CDRL1:
[SEQ ID NO: 84]
SGSSSNIGNNAVN
CDRL2:
[SEQ ID NO: 85]
YDDLLPS
CDRL3:
[SEQ ID NO: 86]
TTWDDSLSGVV
Antibody clone: 2E8-VH
2E8-VH
[SEQ ID NO: 9]
EVQLLESGGGLVQPGGSLRLSCAASGFTFSDYYMSWIRQA
PGKGLEWVSAIGFSDDNTYYADSVKGRFTISRDNSKNTLY
LQMNSLRAEDTAVYYCAGGDGSGWSFWGQGTLVTVSS
2E8-VL
[SEQ ID NO: 33]
QSVLTQPPSASGTPGQRVTISCSGSSSNIGNNAVNWYQQL
PGTAPKLLIYDNNKRPSGVPDRFSGSKSGTSASLAISGLR
SEDEADYYCATWDDSLRGWVFGGGTKLTVLG
CDR regions
CDRH1:
[SEQ ID NO: 87]
DYYMS
CDRH2:
[SEQ ID NO: 88]
AIGFSDDNTYYADSVKG
CDRH3:
[SEQ ID NO: 89]
GDGSGWSF
CDRL1:
[SEQ ID NO: 90]
SGSSSNIGNNAVN
CDRL2:
[SEQ ID NO: 91]
DNNKRPS
CDRL3:
[SEQ ID NO: 92]
ATWDDSLRGWV
Antibody clone: 5C04
5C04-VH
[SEQ ID NO: 10]
EVQLLESGGGLVQPGGSLRLSCAASGFTFSNYGMHWVRQA
PGKGLEWVAVISYDGSNKYYADSVKGRFTISRDNSKNTLY
LQMNSLRAEDTAVYYCAREWRDAFDIWGQGTLVTVSS
5C04-VL
[SEQ ID NO: 34]
QSVLTQPPSASGTPGQRVTISCTGSSSNIGAGYDVHWYQQ
LPGTAPKLLIYSDNQRPSGVPDRFSGSKSGTSASLAISGL
RSEDEADYYCAAWDDSLSGSWVFGGGTKLTVLG
CDR regions
CDRH1:
[SEQ ID NO: 93]
NYGMH
CDRH2:
[SEQ ID NO: 94]
VISYDGSNKYYADSVKG
CDRH3:
[SEQ ID NO: 95]
WRDAFDI
CDRL1:
[SEQ ID NO: 96]
TGSSSNIGAGYDVH
CDRL2:
[SEQ ID NO: 97]
SDNQRPS
CDRL3:
[SEQ ID NO: 98]
AAWDDSLSGSWV
Antibody clone: 5C05
5C05-VH
[SEQ ID NO: 11]
EVQLLESGGGLVQPGGSLRLSCAASGFTFSTYGMHWVRQA
PGKGLEWVAVISYDGSNKYYADSVKGRFTISRDNSKNTLY
LQMNSLRAEDTAVYYCARENFDAFDVWGQGTLVTVSS
5C05-VL
[SEQ ID NO: 35]
QSVLTQPPSASGTPGQRVTISCTGSSSNIGAGYDVHWYQQ
LPGTAPKLLIYSNSQRPSGVPDRFSGSKSGTSASLAISGL
RSEDEADYYCAAWDDSLNGQVVFGGGTKLTVLG
CDR regions
CDRH1:
[SEQ ID NO: 99]
TYGMH
CDRH2:
[SEQ ID NO: 100]
VISYDGSNKYYADSVKG
CDRH3:
[SEQ ID NO: 101]
ENFDAFDV
CDRL1:
[SEQ ID NO: 102]
TGSSSNIGAGYDVH
CDRL2:
[SEQ ID NO: 103]
SNSQRPS
CDRL3:
[SEQ ID NO: 104]
AAWDDSLNGQVV
Antibody clone: 5D07
5D07-VH
[SEQ ID NO: 12]
EVQLLESGGGLVQPGGSLRLSCAASGFTFSTYGMHWVRQA
PGKGLEWVAVIAYDGSKKDYADSVKGRFTISRDNSKNTLY
LQMNSLRAEDTAVYYCAREYRDAFDIWGQGTLVTVSS
5D07-VL
[SEQ ID NO: 36]
QSVLTQPPSASGTPGQRVTISCTGSSSNIGAGYDVHWYQQ
LPGTAPKLLIYGNSNRPSGVPDRFSGSKSGTTASLAISGL
RSEDEADYYCAAWDDSVSGWMFGGGTKLTVLG
CDR regions
CDRH1:
[SEQ ID NO: 105]
TYGMH
CDRH2:
[SEQ ID NO: 106]
VIAYDGSKKDYADSVKG
CDRH3:
[SEQ ID NO: 107]
EYRDAFDI
CDRL1:
[SEQ ID NO: 108]
TGSSSNIGAGYDVH
CDRL2:
[SEQ ID NO: 109]
GNSNRPS
CDRL3:
[SEQ ID NO: 110]
AAWDDSVSGWM
Antibody clone: 5E12
5E12-VH
[SEQ ID NO: 13]
EVQLLESGGGLVQPGGSLRLSCAASGFTFSSYGMHWVRQA
PGKGLEWVAVISYDGINKDYADSMKGRFTISRDNSKNTLY
LQMNSLRAEDTAVYYCARERKDAFDIWGQGTLVTVSS
5E12-VL
[SEQ ID NO: 37]
QSVLTQPPSASGTPGQRVTISCTGSSSNIGAGYDVHWYQQ
LPGTAPKLLIYSNNQRPSGVPDRFSGSKSGTSASLAISGL
RSEDEADYYCATWDDSLNGLVFGGGTKLTVLG
CDR regions
CDRH1:
[SEQ ID NO: 111]
SYGMH
CDRH2:
[SEQ ID NO: 112]
VISYDGINKDYADSMKG
CDRH3:
[SEQ ID NO: 113]
ERKDAFDI
CDRL1:
[SEQ ID NO: 114]
TGSSSNIGAGYDVH
CDRL2:
[SEQ ID NO: 115]
SNNQRPS
CDRL3:
[SEQ ID NO: 116]
ATWDDSLNGLV
Antibody clone: 5G08
5G08-VH
[SEQ ID NO: 14]
EVQLLESGGGLVQPGGSLRLSCAASGFTFNNYGMHWVRQA
PGKGLEWVAVISYDGSNRYYADSVKGRFTMSRDNSKNTLY
LQMNSLRAEDTAVYYCARDRWNGMDVWGQGTLVTVSS
5G08-VL
[SEQ ID NO: 38]
QSVLTQPPSASGTPGQRVTISCSGSSSNIGAGYDVHWYQQ
LPGTAPKLLIYANNQRPSGVPDRFSGSKSGTSASLAISGL
RSEDEADYYCAAWDDSLNGPWVFGGGTKLTVLG
CDR regions
CDRH1:
[SEQ ID NO: 117]
NYGMH
CDRH2:
[SEQ ID NO: 118]
VISYDGSNRYYADSVKG
CDRH3:
[SEQ ID NO: 119]
DRWNGMDV
CDRL1:
[SEQ ID NO: 120]
SGSSSNIGAGYDVH
CDRL2:
[SEQ ID NO: 121]
ANNQRPS
CDRL3:
[SEQ ID NO: 122]
AAWDDSLNGPWV
Antibody clone: 5H06
5H06-VH
[SEQ ID NO: 15]
EVQLLESGGGLVQPGGSLRLSCAASGFTFSSYGMHWVRQA
PGKGLEWVAVISYDGSDTAYADSVKGRFTISRDNSKNTLY
LQMNSLRAEDTAVYYCARDHSVIGAFDIWGQGTLVTVSS
5H06-VL
[SEQ ID NO: 39]
QSVLTQPPSASGTPGQRVTISCSGSSSNIGSNTVNWYQQL
PGTAPKLLIYDNNKRPSGVPDRFSGSKSGTSASLAISGLR
SEDEADYYCSSYAGSNNVVFGGGTKLTVLG
CDR regions
CDRH1:
[SEQ ID NO: 123]
SYGMH
CDRH2:
[SEQ ID NO: 124]
VISYDGSDTAYADSVKG
CDRH3:
[SEQ ID NO: 125]
DHSVIGAFDI
CDRL1:
[SEQ ID NO: 126]
SGSSSNIGSNTVN
CDRL2:
[SEQ ID NO: 127]
DNNKRPS
CDRL3:
[SEQ ID NO: 128]
SSYAGSNNVV
Antibody clone: 6A09
6A09-VH
[SEQ ID NO: 16]
EVQLLESGGGLVQPGGSLRLSCAASGFTFSSYGMHWVRQA
PGKGLEWVAVTSYDGNTKYYANSVKGRFTISRDNSKNTLY
LQMNSLRAEDTAVYYCAREDCGGDCFDYWGQGTLVTVSS
6A09-VL
[SEQ ID NO: 40]
QSVLTQPPSASGTPGQRVTISCTGSSSNIGAGYDVHWYQQ
LPGTAPKLLIYGNSNRPSGVPDRFSGSKSGTSASLAISGL
RSEDEADYYCAAWDDSLNEGVFGGGTKLTVLG
CDR regions
CDRH1:
[SEQ ID NO: 129]
SYGMH
CDRH2:
[SEQ ID NO: 130]
VTSYDGNTKYYANSVKG
CDRH3:
[SEQ ID NO: 131]
EDCGGDCFDY
CDRL1:
[SEQ ID NO: 132]
TGSSSNIGAGYDVH
CDRL2:
[SEQ ID NO: 133]
GNSNRPS
CDRL3:
[SEQ ID NO: 134]
AAWDDSLNEGV
Antibody clone: 6B01
6B01-VH
[SEQ ID NO: 17]
EVQLLESGGGLVQPGGSLRLSCAASGFTFSNYGMHWVRQA
PGKGLEWVAVISYDGSNKYYADSVKGRFTISRDNSKNTLY
LQMNSLRAEDTAVYYCARDQLGEAFDIWGQGTLVTVSS
6B01-VL
[SEQ ID NO: 41]
QSVLTQPPSASGTPGQRVTISCTGSSSNIGAGYDVHWYQQ
LPGTAPKLLIYDNNKRPSGVPDRFSGSKSGTSASLAISGL
RSEDEADYYCATWDDSLSGPVFGGGTKLTVLG
CDR regions
CDRH1:
[SEQ ID NO: 135]
NYGMH
CDRH2:
[SEQ ID NO: 136]
VISYDGSNKYYADSVKG
CDRH3:
[SEQ ID NO: 137]
DQLGEAFDI
CDRL1:
[SEQ ID NO: 138]
TGSSSNIGAGYDVH
CDRL2:
[SEQ ID NO: 139]
DNNKRPS
CDRL3:
[SEQ ID NO: 140]
ATWDDSLSGPV
Antibody clone: 6C11
6C11-VH
[SEQ ID NO: 18]
EVQLLESGGGLVQPGGSLRLSCAASGFTFDDYGMSWVRQA
PGKGLEWVSAISGSGSSTYYADSVKGRFTISRDNSKNTLY
LQMNSLRAEDTAVYYCAGGDIDYFDYWGQGTLVTVSS
6C11-VL
[SEQ ID NO: 42]
QSVLTQPPSASGTPGQRVTISCTGSSSNFGAGYDVHWYQQ
LPGTAPKLLIYENNKRPSGVPDRFSGSKSGTSASLAISGL
RSEDEADYYCAAWDDSLNGPVFGGGTKLTVLG
CDR regions
CDRH1:
[SEQ ID NO: 141]
DYGMS
CDRH2:
[SEQ ID NO: 142]
AISGSGSSTYYADSVKG
CDRH3:
[SEQ ID NO: 143]
GDIDYFDY
CDRL1:
[SEQ ID NO: 144]
TGSSSNFGAGYDVH
CDRL2:
[SEQ ID NO: 145]
ENNKRPS
CDRL3:
[SEQ ID NO: 146]
AAWDDSLNGPV
Antibody clone: 6C12
6C12-VH
[SEQ ID NO: 19]
EVQLLESGGGLVQPGGSLRLSCAASGFTFSSYGMHWVRQA
PGKGLEWVAVISYDGSNKYYADSVKGRFTISRDNSKNTLY
LQMNSLRAEDTAVYYCARERRDAFDIWGQGTLVTVSS
6C12-VL
[SEQ ID NO: 43]
QSVLTQPPSASGTPGQRVTISCTGSSSNIGAGYDVHWYQQ
LPGTAPKLLIYSDNQRPSGVPDRFSGSKSGTSASLAISGL
RSEDEADYYCATWDSDTPVFGGGTKLTVLG
CDR regions
CDRH1:
[SEQ ID NO: 147]
SYGMH
CDRH2:
[SEQ ID NO: 148]
VISYDGSNKYYADSVKG
CDRH3:
[SEQ ID NO: 149]
ERRDAFDI
CDRL1:
[SEQ ID NO: 150]
TGSSSNIGAGYDVH
CDRL2:
[SEQ ID NO: 151]
SDNQRPS
CDRL3:
[SEQ ID NO: 152]
ATWDSDTPV
Antibody clone: 6D01
6D01-VH
[SEQ ID NO: 20]
EVQLLESGGGLVQPGGSLRLSCAASGFTFSSYGMHWVRQA
PGKGLEWVAVISYDGSNKYYADSVKGRFTISRDNSKNTLY
LQMNSLRAEDTAMYYCARDHSAAGYFDYWGQGTLVTVSS
6D01-VL
[SEQ ID NO: 44]
QSVLTQPPSASGTPGQRVTISCSGSSSNIGSNTVNWYQQL
PGTAPKLLIYGNSIRPSGGPDRFSGSKSGTSASLAISGLR
SEDEADYYCASWDDSLSSPVFGGGTKLTVLG
CDR regions
CDRH1:
[SEQ ID NO: 153]
SYGMH
CDRH2:
[SEQ ID NO: 154]
VISYDGSNKYYADSVKG
CDRH3:
[SEQ ID NO: 155]
DHSAAGYFDY
CDRL1:
[SEQ ID NO: 156]
SGSSSNIGSNTVN
CDRL2:
[SEQ ID NO: 157]
GNSIRPS
CDRL3:
[SEQ ID NO: 158]
ASWDDSLSSPV
Antibody clone: 6G03
6G03-VH
[SEQ ID NO: 21]
EVQLLESGGGLVQPGGSLRLSCAASGFTFGSYGMHWVRQA
PGKGLEWVSGISWDSAIIDYAGSVKGRFTISRDNSKNTLY
LQMNSLRAEDTAVYYCAKDEAAAGAFDIWGQGTLVTVSS
6G03-VL
[SEQ ID NO: 45]
QSVLTQPPSASGTPGQRVTISCTGSSSNIGAGYDVHWYQQ
LPGTAPKLLIYGNTDRPSGVPDRFSGSKSGTSASLAISGL
RSEDEADYYCAAWDDSLSGPVVFGGGTKLTVLG
CDR regions
CDRH1:
[SEQ ID NO: 159]
SYGMH
CDRH2:
[SEQ ID NO: 160]
GISWDSAIIDYAGSVKG
CDRH3:
[SEQ ID NO: 161]
DEAAAGAFDI
CDRL1:
[SEQ ID NO: 162]
TGSSSNIGAGYDVH
CDRL2:
[SEQ ID NO: 163]
GNTDRPS
CDRL3:
[SEQ ID NO: 164]
AAWDDSLSGPVV
Antibody clone: 6G08
6G08-VH
[SEQ ID NO: 22]
EVQLLESGGGLVQPGGSLRLSCAASGFTLSSYGISWVRQA
PGKGLEWVSGISGSGGNTYYADSVKGRFTISRDNSKNTLY
LQMNSLRAEDTAVYYCASSVGAYANDAFDIWGQGTLVTVS
S
6G08-VL
[SEQ ID NO: 46]
QSVLTQPPSASGTPGQRVTISCTGSSSNIGAGYDVHWYQQ
LPGTAPKLLIYGDTNRPSGVPDRFSGSKSGTSASLAISGL
RSEDEADYYCAAWDDSLNGPVFGGGTKLTVLG
CDR regions
CDRH1:
[SEQ ID NO: 165]
SYGIS
CDRH2:
[SEQ ID NO: 166]
GISGSGGNTYYADSVKG
CDRH3:
[SEQ ID NO: 167]
SVGAYANDAFDI
CDRL1:
[SEQ ID NO: 168]
TGSSSNIGAGYDVH
CDRL2:
[SEQ ID NO: 169]
GDTNRPS
CDRL3:
[SEQ ID NO: 170]
AAWDDSLNGPV
Antibody clone: 6G11
6G11-VH
[SEQ ID NO: 23]
EVQLLESGGGLVQPGGSLRLSCAASGFTFSSYGMHWVRQA
PGKGLEWMAVISYDGSNKYYADSVKGRFTISRDNSKNTLY
LQMNSLRAEDTAVYYCARELYDAFDIWGQGTLVTVSS
6G11-VL
[SEQ ID NO: 47]
QSVLTQPPSASGTPGQRVTISCTGSSSNIGAGYDVHWYQQ
LPGTAPKLLIYADDHRPSGVPDRFSGSKSGTSASLAISGL
RSEDEADYYCASWDDSQRAVIFGGGTKLTVLG
CDR regions
CDRH1:
[SEQ ID NO: 171]
SYGMH
CDRH2:
[SEQ ID NO: 172]
VISYDGSNKYYADSVKG
CDRH3:
[SEQ ID NO: 173]
ELYDAFDI
CDRL1:
[SEQ ID NO: 174]
TGSSSNIGAGYDVH
CDRL2:
[SEQ ID NO: 175]
ADDHRPS
CDRL3:
[SEQ ID NO: 176]
ASWDDSQRAVI
Antibody clone: 6H08
6H08-VH
[SEQ ID NO: 24]
EVQLLESGGGLVQPGGSLRLSCAASGFTFNNYGMHWVRQA
PGKGLEWVAVISYDGSNKYYADSVKGRFTISKDNSKNTLY
LQMNSLRAEDTAVYYCAREYKDAFDIWGQGTLVTVSS
6H08-VL
[SEQ ID NO: 48]
QSVLTQPPSASGTPGQRVTISCTGSSSNIGSNTVNWYQQL
PGTAPKLLIYDNNKRPSGVPDRFSGSKSGTSASLAISGLR
SEDEADYYCQAWGTGIRVFGGGTKLTVLG
CDR regions
CDRH1:
[SEQ ID NO: 177]
NYGMH
CDRH2:
[SEQ ID NO: 178]
VISYDGSNKYYAD SVKG
CDRH3:
[SEQ ID NO: 179]
EYKDAFDI
CDRL1:
[SEQ ID NO: 180]
TGSSSNIGSNTVN
CDRL2:
[SEQ ID NO: 181]
DNNKRPS
CDRL3:
[SEQ ID NO: 182]
QAWGTGIRV
Antibody clone: 7C07
7C07-VH
[SEQ ID NO: 25]
EVQLLESGGGLVQPGGSLRLSCAASGFTFSSYGMHWVRQA
PGKGLEWVAVISYDGSNKYYADSVKGRFTISRDNSQNTLY
LQMNSLRAEDTAVYYCAREFGYIILDYWGQGTLVTVSS
7C07-VL
[SEQ ID NO: 49]
QSVLTQPPSASGTPGQRVTISCSGSSSNIGSNTVNWYQQL
PGTAPKLLIYRDYERPSGVPDRFSGSKSGTSASLAISGLR
SEDEADYYCMAWDDSLSGVVFGGGTKLTVLG
CDR regions
CDRH1:
[SEQ ID NO: 183]
SYGMH
CDRH2:
VISYDGSNKYYADSVKG
[SEQ ID NO: 184]
CDRH3:
[SEQ ID NO: 185]
EFGYIILDY
CDRL1:
[SEQ ID NO: 186]
SGSSSNIGSNTVN
CDRL2:
[SEQ ID NO: 187]
RDYERPS
CDRL3:
[SEQ ID NO: 188]
MAWDDSLSGVV
Antibody clone: 4B02
4B02-VH
[SEQ ID NO: 26]
EVQLLESGGGLVQPGGSLRLSCAASGFTFSNHGMHWVRQA
PGKGLEWVAVISYDGTNKYYADSVRGRFTISRDNSKNTLY
LQMNSLRAEDTAVYYCARETWDAFDVWGQGTLVTVSS
4B02-VL
[SEQ ID NO: 50]
QSVLTQPPSASGTPGQRVTISCSGSSSNIGSNNANWYQQL
PGTAPKLLIYDNNKRPSGVPDRFSGSKSGTSASLAISGLR
SEDEADYYCQAWDSSTVVFGGGTKLTVLG
CDR regions
CDRH1:
[SEQ ID NO: 189]
NHGMH
CDRH2:
[SEQ ID NO: 190]
VISYDGTNKYYADSVRG
CDRH3:
[SEQ ID NO: 191]
ETWDAFDV
CDRL1:
[SEQ ID NO: 192]
SGSSSNIGSNNAN
CDRL2:
[SEQ ID NO: 193]
DNNKRPS
CDRL3:
[SEQ ID NO: 194]
QAWDSSTVV

Therefore, in some embodiments, the first antibody molecule may comprise a variable heavy chain (VH) comprising the following CDRs:
(i) SEQ ID NO: 51 and SEQ ID NO: 52 and SEQ ID NO: 53; or
(ii) SEQ ID NO: 57 and SEQ ID NO: 58 and SEQ ID NO: 59; or
(iii) SEQ ID NO: 63 and SEQ ID NO: 64 and SEQ ID NO: 65; or
(iv) SEQ ID NO: 69 and SEQ ID NO: 70 and SEQ ID NO: 71; or
(v) SEQ ID NO: 75 and SEQ ID NO: 76 and SEQ ID NO: 77; or
(vi) SEQ ID NO: 81 and SEQ ID NO: 82 and SEQ ID NO: 83; or
(vii) SEQ ID NO: 87 and SEQ ID NO: 88 and SEQ ID NO: 89; or
(viii) SEQ ID NO: 93 and SEQ ID NO: 94 and SEQ ID NO: 95; or
(ix) SEQ ID NO: 99 and SEQ ID NO: 100 and SEQ ID NO: 101; or
(x) SEQ ID NO: 105 and SEQ ID NO: 106 and SEQ ID NO: 107; or
(xi) SEQ ID NO: 111 and SEQ ID NO: 112 and SEQ ID NO: 113; or
(xii) SEQ ID NO: 117 and SEQ ID NO: 118 and SEQ ID NO: 119; or
(xiii) SEQ ID NO: 123 and SEQ ID NO: 124 and SEQ ID NO: 125; or
(xiv) SEQ ID NO: 129 and SEQ ID NO: 130 and SEQ ID NO: 131; or
(xv) SEQ ID NO: 135 and SEQ ID NO: 136 and SEQ ID NO: 137; or
(xvi) SEQ ID NO: 141 and SEQ ID NO: 142 and SEQ ID NO: 143; or
(xvii) SEQ ID NO: 147 and SEQ ID NO: 148 and SEQ ID NO: 149; or
(xviii) SEQ ID NO: 153 and SEQ ID NO: 154 and SEQ ID NO: 155; or
(xix) SEQ ID NO: 159 and SEQ ID NO: 160 and SEQ ID NO: 161; or
(xx) SEQ ID NO: 165 and SEQ ID NO: 166 and SEQ ID NO: 167; or
(xxi) SEQ ID NO: 171 and SEQ ID NO: 172 and SEQ ID NO: 173; or
(xxii) SEQ ID NO: 177 and SEQ ID NO: 178 and SEQ ID NO: 179; or
(xxiii) SEQ ID NO: 183 and SEQ ID NO: 184 and SEQ ID NO: 185; or
(xxiv) SEQ ID NO: 189 and SEQ ID NO: 190 and SEQ ID NO: 191.

In some additional or alternative embodiments, the first antibody molecule comprises a variable light chain (VL) comprising the following CDRs:
(i) SEQ ID NO: 54 and SEQ ID NO: 55 and SEQ ID NO: 56; or
(ii) SEQ ID NO: 60 and SEQ ID NO: 61 and SEQ ID NO: 62; or
(iii) SEQ ID NO: 66 and SEQ ID NO: 67 and SEQ ID NO: 68; or
(iv) SEQ ID NO: 72 and SEQ ID NO: 73 and SEQ ID NO: 74; or
(v) SEQ ID NO: 78 and SEQ ID NO: 79 and SEQ ID NO: 80; or
(vi) SEQ ID NO: 84 and SEQ ID NO: 85 and SEQ ID NO: 86; or
(vii) SEQ ID NO: 90 and SEQ ID NO: 91 and SEQ ID NO: 92; or
(viii) SEQ ID NO: 96 and SEQ ID NO: 97 and SEQ ID NO: 98; or
(ix) SEQ ID NO: 102 and SEQ ID NO: 103 and SEQ ID NO: 104; or
(x) SEQ ID NO: 108 and SEQ ID NO: 109 and SEQ ID NO: 110; or
(xi) SEQ ID NO: 114 and SEQ ID NO: 115 and SEQ ID NO: 116; or
(xii) SEQ ID NO: 120 and SEQ ID NO: 121 and SEQ ID NO: 122; or
(xiii) SEQ ID NO: 126 and SEQ ID NO: 127 and SEQ ID NO: 128; or
(xiv) SEQ ID NO: 132 and SEQ ID NO: 133 and SEQ ID NO: 134; or
(xv) SEQ ID NO: 138 and SEQ ID NO: 139 and SEQ ID NO: 140; or
(xvi) SEQ ID NO: 144 and SEQ ID NO: 145 and SEQ ID NO: 146; or
(xvii) SEQ ID NO: 150 and SEQ ID NO: 151 and SEQ ID NO: 152; or
(xviii) SEQ ID NO: 156 and SEQ ID NO: 157 and SEQ ID NO: 158; or
(xix) SEQ ID NO: 162 and SEQ ID NO: 163 and SEQ ID NO: 164; or
(xx) SEQ ID NO: 168 and SEQ ID NO: 169 and SEQ ID NO: 170; or
(xxi) SEQ ID NO: 174 and SEQ ID NO: 175 and SEQ ID NO: 176; or
(xxii) SEQ ID NO: 180 and SEQ ID NO: 181 and SEQ ID NO: 182; or
(xxiii) SEQ ID NO: 186 and SEQ ID NO: 187 and SEQ ID NO: 188; or
(xxiv) SEQ ID NO: 192 and SEQ ID NO: 193 and SEQ ID NO: 194.

In some additional or alternative embodiments, the first antibody molecule comprises a variable heavy chain (VH) amino acid sequence selected from the group consisting of: SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; and SEQ ID NO: 26.

In some additional or alternative embodiments, the first antibody molecule comprises a variable light chain (VL) amino acid sequence selected from the group consisting of: SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 38; SEQ ID NO: 39; SEQ ID NO: 40; SEQ ID NO: 41; SEQ ID NO: 42; SEQ ID NO: 43; SEQ ID NO: 44; SEQ ID NO: 45; SEQ ID NO: 46; SEQ ID NO: 47; SEQ ID NO: 48; SEQ ID NO: 49; and SEQ ID NO: 50.

In some additional or alternative embodiments, the first antibody molecule comprises the following CDR amino acid sequences:
(i) SEQ ID NO: 51 and SEQ ID NO: 52 and SEQ ID NO: 53 and SEQ ID NO: 54 and SEQ ID NO: 55 and SEQ ID NO: 56; or
(ii) SEQ ID NO: 57 and SEQ ID NO: 58 and SEQ ID NO: 59 and SEQ ID NO: 60 and SEQ ID NO: 61 and SEQ ID NO: 62; or
(iii) SEQ ID NO: 63 and SEQ ID NO: 64 and SEQ ID NO: 65 and SEQ ID NO: 66 and SEQ ID NO: 67 and SEQ ID NO: 68; or
(iv) SEQ ID NO: 69 and SEQ ID NO: 70 and SEQ ID NO: 71 and SEQ ID NO: 72 and SEQ ID NO: 73 and SEQ ID NO: 74; or
(v) SEQ ID NO: 75 and SEQ ID NO: 76 and SEQ ID NO: 77 and SEQ ID NO: 78 and SEQ ID NO: 79 and SEQ ID NO: 80; or
(vi) SEQ ID NO: 81 and SEQ ID NO: 82 and SEQ ID NO: 83 and SEQ ID NO: 84 and SEQ ID NO: 85 and SEQ ID NO: 86; or
(vii) SEQ ID NO: 87 and SEQ ID NO: 88 and SEQ ID NO: 89 and SEQ ID NO: 90 and SEQ ID NO: 91 and SEQ ID NO: 92; or
(viii) SEQ ID NO: 93 and SEQ ID NO: 94 and SEQ ID NO: 95 and SEQ ID NO: 96 and SEQ ID NO: 97 and SEQ ID NO: 98; or
(ix) SEQ ID NO: 99 and SEQ ID NO: 100 and SEQ ID NO: 101 and SEQ ID NO: 102 and SEQ ID NO: 103 and SEQ ID NO: 104; or
(x) SEQ ID NO: 105 and SEQ ID NO: 106 and SEQ ID NO: 107 and SEQ ID NO: 108 and SEQ ID NO: 109 and SEQ ID NO: 110; or
(xi) SEQ ID NO: 111 and SEQ ID NO: 112 and SEQ ID NO: 113 and SEQ ID NO: 114 and SEQ ID NO: 115 and SEQ ID NO: 116; or
(xii) SEQ ID NO: 117 and SEQ ID NO: 118 and SEQ ID NO: 119 and SEQ ID NO: 120 and SEQ ID NO: 121 and SEQ ID NO: 122; or
(xiii) SEQ ID NO: 123 and SEQ ID NO: 124 and SEQ ID NO: 125 and SEQ ID NO: 126 and SEQ ID NO: 127 and SEQ ID NO: 128; or
(xiv) SEQ ID NO: 129 and SEQ ID NO: 130 and SEQ ID NO: 131 and SEQ ID NO: 132 and SEQ ID NO: 133 and SEQ ID NO: 134; or
(xv) SEQ ID NO: 135 and SEQ ID NO: 136 and SEQ ID NO: 137 and SEQ ID NO: 138 and SEQ ID NO: 139 and SEQ ID NO: 140; or
(xvi) SEQ ID NO: 141 and SEQ ID NO: 142 and SEQ ID NO: 143 and SEQ ID NO: 144 and SEQ ID NO: 145 and SEQ ID NO: 146; or
(xvii) SEQ ID NO: 147 and SEQ ID NO: 148 and SEQ ID NO: 149 and SEQ ID NO: 150 and SEQ ID NO: 151 and SEQ ID NO: 152; or
(xviii) SEQ ID NO: 153 and SEQ ID NO: 154 and SEQ ID NO: 155 and SEQ ID NO: 156 and SEQ ID NO: 157 and SEQ ID NO: 158; or
(xix) SEQ ID NO: 159 and SEQ ID NO: 160 and SEQ ID NO: 161 and SEQ ID NO: 162 and SEQ ID NO: 163 and SEQ ID NO: 164; or
(xx) SEQ ID NO: 165 and SEQ ID NO: 166 and SEQ ID NO: 167 and SEQ ID NO: 168 and SEQ ID NO: 169 and SEQ ID NO: 170; or
(xxi) SEQ ID NO: 171 and SEQ ID NO: 172 and SEQ ID NO: 173 and SEQ ID NO: 174 and SEQ ID NO: 175 and SEQ ID NO: 176; or
(xxii) SEQ ID NO: 177 and SEQ ID NO: 178 and SEQ ID NO: 179 and SEQ ID NO: 180 and SEQ ID NO: 181 and SEQ ID NO: 182; or
(xxiii) SEQ ID NO: 183 and SEQ ID NO: 184 and SEQ ID NO: 185 and SEQ ID NO: 186 and SEQ ID NO: 187 and SEQ ID NO: 188; or
(xxiv) SEQ ID NO: 189 and SEQ ID NO: 190 and SEQ ID NO: 191 and SEQ ID NO: 192 and SEQ ID NO: 193 and SEQ ID NO: 194.

In some additional or alternative embodiments, the first antibody molecule comprises the following amino acid sequences:
(i) SEQ ID NO: 3 and SEQ ID NO: 27; or
(ii) SEQ IS NO: 4 and SEQ ID NO: 28; or
(iii) SEQ IS NO: 5 and SEQ ID NO: 29; or
(iv) SEQ ID NO: 6 and SEQ ID NO: 30; or
(v) SEQ ID NO: 7 and SEQ ID NO: 31; or
(vi) SEQ ID NO: 8 and SEQ ID NO: 32; or
(vii) SEQ ID NO: 9 and SEQ ID NO: 33; or
(viii) SEQ ID NO: 10 and SEQ ID NO: 34; or
(ix) SEQ ID NO: 11 and SEQ ID NO: 35; or
(x) SEQ ID NO: 12 and SEQ ID NO: 36; or
(xi) SEQ ID NO: 13 and SEQ ID NO: 37; or
(xii) SEQ ID NO: 14 and SEQ ID NO: 38; or
(xiii) SEQ ID NO: 15 and SEQ ID NO: 39; or
(xiv) SEQ ID NO: 16 and SEQ ID NO: 40; or
(xv) SEQ ID NO: 17 and SEQ ID NO: 41; or
(xvi) SEQ ID NO: 18 and SEQ ID NO: 42; or
(xvii) SEQ ID NO: 19 and SEQ ID NO: 43; or
(xviii) SEQ ID NO: 20 and SEQ ID NO: 44; or
(xix) SEQ ID NO: 21 and SEQ ID NO: 45; or
(xx) SEQ ID NO: 22 and SEQ ID NO: 46; or
(xxi) SEQ ID NO: 23 and SEQ ID NO: 47; or
(xxii) SEQ ID NO: 24 and SEQ ID NO: 48; or
(xxiii) SEQ ID NO: 25 and SEQ ID NO: 49; or
(xxiv) SEQ ID NO: 26 and SEQ ID NO: 50.

In some embodiments, which are sometimes preferred embodiments, the antibody molecule that specifically binds FcγRIIb comprises the following CDR regions: SEQ ID NO: 171 (CDRH1), SEQ ID NO: 172 (CDRH2), SEQ ID NO: 173 (CDRH3), SEQ ID NO: 174 (CDRL1), SEQ ID NO: 175 (CDRL2) and SEQ ID NO: 176 (CDRL3), i.e. the CDR regions of clone 6G11.

In some embodiments, which are sometimes preferred embodiments, the antibody molecule that specifically binds FcγRIIb comprises the following constant regions: SEQ ID NO: 1 (CH) and SEQ ID NO: 2 (CL) and the following variable regions: SEQ ID NO: 23 (VL) and SEQ ID NO: 47 (VH) i.e. the constant and variable regions of clone 6G11, which antibody molecule has further been modified to have reduced binding to Fcγ receptors via its Fc region. In some embodiments, which are sometimes preferred embodiments, the antibody molecule that specifically binds FcγRIIb comprises the following constant regions: SEQ ID NO: 195 (CH) and SEQ ID NO: 2 (CL) and the following variable regions: SEQ ID NO: 23 (VL) and SEQ ID NO: 47 (VH) i.e. the constant and variable regions of clone 6G11 including the N297Q mutation.

As defined herein in the first to seventh aspects, the second antibody molecule specifically binds to PD-1 or PD-L1. The third antibody molecule as defined herein specifically binds to CTLA-4 and binds to at least one Fcγ receptor via its Fc region. In some embodiments, the antibody molecule that specifically binds to PD-1 also binds to at least one Fcγ receptor via its Fc region. In some embodiments, the antibody molecule that specifically binds to PD-L1 also binds to at least one Fcγ receptor via its Fc region.

The second antibody molecule may specifically bind to programmed death-ligand 1 (PD-L1), also known as CD274 or B7 homolog 1 (B7-H1).

In some embodiments, the antibody molecule that specifically binds to PD-L1 is selected from one or more of the following, non-limiting examples of anti-PD-L1 antibodies: Atezolizumab (currently approved for use), Durvalumab (currently approved for use), Avelumab (currently approved for use), CS1001 (currently in clinical development), KN035 (Envafolimab)—a PD-L1 antibody with subcutaneous formulation currently under clinical evaluations in the US, China, and Japan, and CK-301 (currently in clinical development by Checkpoint Therapeutics).

In a preferred embodiment, the antibody that binds specifically to PD-L1 is Atezolizumab, Durvalumab, or Avelumab. In some embodiments, the antibody that binds specifically to PD-L1 is a combination of two or more of these antibodies.

In alternative or additional embodiments, the second antibody molecule may bind specifically to programmed cell death-protein 1 (PD1), also known as CD 279.

In some embodiments, the antibody molecule that specifically binds to PD-1 is selected from one or more of the following, non-limiting examples of anti-PD-1 antibodies: Pembrolizumab (currently approved for use), Nivolumab (currently approved for use), Cemiplimab (currently approved for use), Camrelizumab (currently approved for use), Spartalizumab (currently in clinical development), Dostarlimab (currently in clinical development), Tislelizumab (currently in clinical development), JTX-4014 (currently in clinical development), Sintilimab (IBI308) (currently in clinical development), Toripalimab (JS 001) (currently in clinical development), AMP-224 (currently in clinical development), and AMP-514 (MEDI0680) (currently in clinical development).

In a preferred embodiment, the antibody that binds specifically to PD-1 is Pembrolizumab, Nivolumab, Cemiplimab, or Camrelizumab. In some embodiments, the antibody that binds specifically to PD-1 is a combination of two or more of these antibodies. In a preferred embodiment, the antibody that binds specifically to PD-1 is Pembrolizumab.

The third antibody molecule specifically binds to CTLA-4. CTLA-4, or CTLA4, which stands for cytotoxic T-lymphocyte-associate protein 4, is also known as CD152. It is a protein receptor, that functioning as an immune checkpoint, downregulates immune responsive. CTLA4 is constitutively expressed in regulatory T cells but only upregulated in conventional T cells after activation—a phenomenon which is particularly notable in cancers. In some embodiments, the third antibody molecule is ipilimumab (such as Yervoy® from Bristol-Myers Squibb). In some embodiments the third antibody molecule is tremelimumab (formerly denoted ticilimumab and, CP-675,206), which is a fully human monoclonal antibody against CTLA-4, previously in development by Pfizer and now in clinical development by MedImmune. In a preferred embodiment, the antibody that binds specifically to CTLA-4 is ipilimumab.

Checkpoint inhibitory receptors CTLA-4 and PD-1/PD-L1 function to limit T cell activation and proliferation, and as such are important in controlling immune homeostasis and prevention of reaction against self Δt the same time, tumors may circumvent immune attack, by release of soluble factors or through cognate interactions, that upregulate and/or engage these inhibitory immune receptors limiting T cell activation and proliferation. For example, tumors in response to exposure of interferon-gamma may upregulate PD-L1, which upon ligation of PD-1 molecules (on effector T cells) reduces effector T cell activation, proliferation, and ultimately effector T cell-mediated antitumor immunity. Tumor release of other factors e.g. cytokines or chemokines may promote maturation of e.g. tumor-associated macrophages, myeloid-derived suppressor cells or T regulatory cells with concomitant upregulation of immune inhibitory receptors which, when ligating CTLA-4 or PD-1 on effector T cells, limit T cell proliferation and activation, reducing T cell mediated anti-tumor immunity.

Accordingly, one mechanism by which antibodies to CTLA-4, PD-1 and PD-L1 may increase antitumor activity is by blocking CTLA-4 and/or PD-1 interactions with their natural ligands, and associated inhibitory signalling in effector T cells, which may be CD8+ or CD4+.

In contrast, FcγR differently modulate therapeutic activity of antibodies to CTLA-4, PD-1 and PD-L1. While therapeutic activity of anti-CTLA-4 antibodies is enhanced by their engagement of FcγRs [18, 21], anti-PD-1 antibodies activity is hampered by FcγR-engagement [19, 20] and published patent application WO 2021/009358. Tumor microenvironment context-dependent enhancement of anti-PD-L1 antibodies therapeutic activity by FcγRs have been described [19][22].

While mechanisms governing FcγR modulation of in particular anti-PD-1 and anti-PD-L1 antibodies are incompletely characterized, available data suggests that FcγR-engagement is beneficial for antibodies whose targets are sufficiently highly expressed on immune suppressive cells, but not on immune effector cells, to trigger FcγR-mediated target cell depletion. For example, CTLA-4 is highly expressed and higher expressed on intratumoral Tregs compared to effector T cells. Accordingly, in FcγR-humanized mice FcγR-engaging anti-CTLA-4 antibodies of e.g. human IgG1 isotype efficiently depleted Treg but not CD8+ effector cells [18]. Further consistent with a positive role for FcγRs in therapeutic activity of anti-CTLA-4 antibodies, melanoma patients carrying high affinity SNPs of FcγRIIIa showed improved survival compared with patients expressing lower affinity SNPs when treated with the human FcγR-engaging IgG1 anti-CTLA-4 antibody ipilimumab.

Conversely, PD-1 is highly expressed on effector CD8+ T cells [19], including human intratumoral CD8+ T cells (see, for example, published patent application WO 2021/009358) and may be higher expressed on effector compared with (immune suppressive) Treg cells [19]. Whether by mechanisms of FcγR-dependent depletion of antibody-coated PD-1 expressing CD8+ T cell [19] or FcγR-dependent transfer of anti-PD-1 antibodies from CD8+ T cell to tumor-associated macrophages [20], FcγRs have been shown to reduce efficacy of anti-PD-1 antibodies in vivo. Relevance to the human clinical setting is provided by in vitro mechanistic studies on clinically relevant nivolumab and pembrolizumab anti-PD-1 antibodies, human FcγR-expressing macrophages and human T cells expressing PD-1 at levels relevant to the human intratumoral setting ([20] and published patent application WO 2021/009358).

The second antibody molecule may also bind via its Fc region to a at least one Fcγ receptor. The third antibody molecule binds via its Fc region to a at least one Fcγ receptor.

As discussed above, Fcγ receptors are present on immune effector cells. The at least one Fcγ receptor may be present on the same immune effector cell as the FcγRIIb to which the first antibody molecule binds and/or it may be an Fcγ receptor present on another immune effector cell.

The immune effector cell may include, but is not limited to, the following: macrophages, neutrophils, monocytes, natural killer (NK) cells, basophils, eosinophils, mast cells, platelets, cytotoxic T cells, and memory T cells. In some preferred embodiments, the immune effector cell is a macrophage.

In some embodiments, the second antibody molecule and/or the third antibody molecule is selected from the group consisting of a human antibody molecule, a humanized antibody molecule, and an antibody molecule of human origin.

In some additional or alternative embodiments, the second antibody molecule and/or third antibody molecule is a monoclonal antibody molecule or an antibody molecule of monoclonal origin.

In some additional or alternative embodiments, the second antibody molecule and/or third antibody molecule is selected from the group consisting of a full-size antibody, a chimeric antibody, a single chain antibody, and an antigen-binding fragment thereof retaining the ability to bind an Fcγ receptor via its Fc region.

In some additional or alternative embodiments, the second antibody molecule and/or third antibody molecule is a human IgG antibody, a humanized IgG antibody molecule or an IgG antibody molecule of human origin.

The Fcγ receptor bound specifically by the Fc region of the third antibody molecule (and in some embodiments, the second antibody molecule) may, in some preferred embodiments, be an activating Fcγ receptor as described herein. This allows activation of effector cell functions and thereby trigger mechanisms such as antibody-dependent cellular cytotoxicity (ADCC), antibody dependent cellular phagocytosis (ADCP), cytokine release, and/or antibody dependent endocytosis, as well as NETosis (i.e. activation and release of NETs, Neutrophil extracellular traps) in the case of neutrophils. Antibody binding to an activating Fcγ receptor can also lead to an increase in certain activation markers, such as CD40, MHCII, CD38, CD80 and/or CD86.

In some embodiments, the second antibody molecule and/or third antibody molecule is engineered for improved binding to activating Fc gamma receptors. For instance, in order to be able to bind to an activating Fcγ receptor, the Fc region of the second antibody molecule and/or the third antibody can, in some embodiments, be glycosylated at position 297. The carbohydrate residue in this position helps binding to Fcγ receptors. In some embodiments it is preferred that these residues are biantennary carbohydrates which contain GlnNAc, mannose, with terminal galactose residues and sialic acid. It should contain the CH₂ part of the Fc molecule. In a preferred embodiment, the second antibody molecule specifically binds PD-L1, and is engineered for improved binding to activating Fc gamma receptors.

In other embodiments the second antibody may be engineered for reduced binding to FcγRs, e.g. the anti-PD-1 antibody tislelizumab (Beigene; IgG4 S228P, E233P, F234V, L235A, D265A, R409K) and/or the anti-PD-L1 antibody atezolizumab (Roche/Genentech; IgG1 N297A).

The combination of a first antibody molecule, a second antibody molecule, and a third antibody molecule described herein can be used use in the treatment of cancer in a patient.

The pharmaceutical composition and kit comprising the first antibody molecule, second antibody molecule, and third antibody molecule disclosed herein are also suitable for use in the treatment of cancer in a patient, and the following embodiments will also be understood to apply to such uses of the pharmaceutical compositions and kits disclosed herein.

“Patient” (or “subject”) as the term is used herein refers to an animal, including human, that has been diagnosed as having cancer or has been identified as likely to have cancer and/or that exhibits symptoms of cancer. We include that the cancer is an FcγRIIb negative cancer or a cancer that is considered as likely to be FcγRIIb negative cancer. We also include that the cancer is an FcγRIIb positive cancer or a cancer that is considered as likely to be FcγRIIb positive cancer.

We include that the patient could be mammalian or non-mammalian. Preferably, the patient is a human or is a mammalian, such as a horse, or a cow, or a sheep, or a pig, or a camel, or a dog, or a cat. Most preferably, the mammalian patient is a human.

By “exhibits”, we include that the subject displays a cancer symptom and/or a cancer diagnostic marker, and/or the cancer symptom and/or a cancer diagnostic marker can be measured, and/or assessed, and/or quantified.

It would be readily apparent to the person skilled in medicine what the cancer symptoms and cancer diagnostic markers would be and how to measure and/or assess and/or quantify whether there is a reduction or increase in the severity of the cancer symptoms, or a reduction or increase in the cancer diagnostic markers; as well as how those cancer symptoms and/or cancer diagnostic markers could be used to form a prognosis for the cancer.

Cancer treatments are often administered as a course of treatment, which is to say that the therapeutic agent is administered over a period of time. The length of time of the course of treatment will depend on a number of factors, which could include the type of therapeutic agent being administered, the type of cancer being treated, the severity of the cancer being treated, and the age and health of the patient, amongst others reasons.

In some embodiments, the cancer is a FcγRIIb-positive B-cell cancer. By “FcγRIIb-positive cancer”, we include any cancer that expresses Fc RIIB, albeit at different levels. Fc RIIB expression is most pronounced in chronic lymphocytic leukaemia and mantle cell lymphomas, moderately so in diffuse large B cell lymphoma and least pronounced in follicular lymphomas. However, in some cases subjects with cancers that generally express low levels of FcγRIIB (e.g. follicular lymphomas) may have very high levels of Fc RIIB expression. The expression level of Fc RIIB in different types of B cell cancer (and, in particular, those mentioned above) correlates with rate of internalization of the antibody molecule Rituximab. Therefore, the expression of FcγRIIB and the associated internalization of antibody molecules is believed to be a common mechanism that is shared by B cell cancers (Lim et al., 2011, Blood, 118(9):2530-40). The FcγRIIB-dependent initialization of an antibody molecule can be blocked by herein disclosed antibodies to FcγRIIB.

Accordingly, the combinations of antibodies disclosed herein may be used in treating B cell cancers, and, in particular, relapsed mantle cell lymphoma and/or refractory mantle cell lymphoma, and/or relapsed follicular lymphoma and/or refractory follicular lymphoma, and/or relapsed diffuse large B cell lymphoma and/or refractory diffuse large B cell lymphoma.

In some other embodiments, which may be more preferred, the cancer is a FcγRIIb negative cancer. By “FcγRIIb negative cancer” we include any cancer that does not present any FcγRIIb receptors. This can be tested using anti-FcγRIIB specific antibodies in a variety of methods including immunohistochemistry and flow cytometry such as indicated in Tutt et al, J Immunol, 2015, 195 (11) 5503-5516.

In some preferred embodiments, the cancer is selected from the group consisting of carcinomas, sarcomas, and lymphomas.

In some embodiments, the cancer is a carcinoma selected from the group consisting of adenocarcinoma, squamous cell carcinoma, adenosquamous carcinoma, anaplastic or undifferentiated carcinoma, large cell carcinoma and small cell carcinoma.

In some embodiments, the cancer is a sarcoma selected from the group consisting of osteosarcoma, chondrosarcoma, liposarcoma, and leiomyosarcoma.

FcγRIIb negative cancer may be selected from the group consisting of melanoma, breast cancer, ovarian cancer, cervical cancer, prostate cancer, metastatic hormone-refractory prostate cancer, colorectal cancer, lung cancer, small cell lung carcinoma, small cell lung cancer (SCLC), non-small cell lung cancer, urothelial carcinoma, bladder cancer, kidney cancer, mesothelioma, Merkel cell carcinoma, head and neck cancer, and pancreatic cancer.

In additional or alternative embodiments, the cancer as described herein is intended to include any cancer where treatment with antibodies that specifically bind to CTLA-4 and/or PD-1 and/or PD-L1 are indicated. By “indicated” we mean where said antibodies have been approved for use in treatment of said cancers, or have been used in clinical trials against these cancers, or have been suggested as potentially useful in treating these cancers (e.g. from in vivo animal models or in vitro studies).

Each one of the above described cancers is well-known, and the symptoms and cancer diagnostic markers are well described, as are the therapeutic agents used to treat those cancers. Accordingly, the symptoms, cancer diagnostic markers, and therapeutic agents used to treat the above mentioned cancer types would be known to those skilled in medicine.

Clinical definitions of the diagnosis, prognosis and progression of a large number of cancers rely on certain classifications known as staging. Those staging systems act to collate a number of different cancer diagnostic markers and cancer symptoms to provide a summary of the diagnosis, and/or prognosis, and/or progression of the cancer. It would be known to the person skilled in oncology how to assess the diagnosis, and/or prognosis, and/or progression of the cancer using a staging system, and which cancer diagnostic markers and cancer symptoms should be used to do so.

By “cancer staging”, we include the Rai staging, which includes stage 0, stage I, stage II, stage III and stage IV, and/or the Binet staging, which includes stage A, stage B and stage C, and/or the Ann Arbour staging, which includes stage I, stage II, stage III and stage IV.

It is known that cancer can cause abnormalities in the morphology of cells. These abnormalities often reproducibly occur in certain cancers, which means that examining these changes in morphology (otherwise known as histological examination) can be used in the diagnosis or prognosis of cancer. Techniques for visualizing samples to examine the morphology of cells, and preparing samples for visualization, are well known in the art; for example, light microscopy or confocal microscopy.

By “histological examination”, we include the presence of small, mature lymphocyte, and/or the presence of small, mature lymphocytes with a narrow border of cytoplasm, the presence of small, mature lymphocytes with a dense nucleus lacking discernible nucleoli, and/or the presence of small, mature lymphocytes with a narrow border of cytoplasm, and with a dense nucleus lacking discernible nucleoli, and/or the presence of atypical cells, and/or cleaved cells, and/or prolymphocytes.

It is well known that cancer is a result of mutations in the DNA of the cell, which can lead to the cell avoiding cell death or uncontrollably proliferating. Therefore, examining these mutations (also known as cytogenetic examination) can be a useful tool for assessing the diagnosis and/or prognosis of a cancer. An example of this is the deletion of the chromosomal location 13q14.1 which is characteristic of chronic lymphocytic leukaemia. Techniques for examining mutations in cells are well known in the art; for example, fluorescence in situ hybridization (FISH).

By “cytogenetic examination”, we include the examination of the DNA in a cell, and, in particular the chromosomes. Cytogenetic examination can be used to identify changes in DNA which may be associated with the presence of a refractory cancer and/or relapsed cancer. Such may include: deletions in the long arm of chromosome 13, and/or the deletion of chromosomal location 13q14.1, and/or trisomy of chromosome 12, and/or deletions in the long arm of chromosome 12, and/or deletions in the long arm of chromosome 11, and/or the deletion of 11 q, and/or deletions in the long arm of chromosome 6, and/or the deletion of 6q, and/or deletions in the short arm of chromosome 17, and/or the deletion of 17p, and/or the t(11:14) translocation, and/or the (ql3:q32) translocation, and/or antigen gene receptor rearrangements, and/or BCL2 rearrangements, and/or BCL6 rearrangements, and/or t(14:18) translocations, and/or t(11:14) translocations, and/or (ql3:q32) translocations, and/or (3:v) translocations, and/or (8:14) translocations, and/or (8:v) translocations, and/or t(11:14) and (ql3:q32) translocations.

It is known that patients with cancer exhibit certain physical symptoms, which are often as a result of the burden of the cancer on the body. Those symptoms often reoccur in the same cancer, and so can be characteristic of the diagnosis, and/or prognosis, and/or progression of the disease. A person skilled in medicine would understand which physical symptoms are associated with which cancers, and how assessing those physical systems can correlate to the diagnosis, and/or prognosis, and/or progression of the disease. By “physical symptoms”, we include hepatomegaly, and/or splenomegaly.

The combinations, uses, methods, pharmaceutical compositions and kits described herein are useful in the treatment of a cancer that is resistant to treatment with an antibody molecule that specifically binds to PD-1 or PD-L1, and/or an antibody molecule that specifically binds to CTLA-4.

By “resistant to treatment” we mean that the patient has a reduced level of responsiveness to treatment with an antibody molecule that specifically binds to PD-1 or PD-L1, and/or an antibody molecule that specifically binds to CTLA-4, compared to a previous level of responsiveness or expected level of responsiveness, or a level of responsiveness seen when treating other types of cancer. This includes the situation where a patient has previously been treated with said antibody molecules (i.e. they have acquired resistance), and also includes the situation where the patient has never been treated with said antibody molecules (i.e. they are inherently resistant).

Resistance to treatment can be measured in a variety of ways, for instance, by monitoring the patient to ensure that the cancer is receding in the expected way, and identifying patients not responding at all to the treatment. For example, resistance to treatment may be measured using an immunoscore test, as is known in the art and described herein.

By “resistant to treatment” we also include types of cancer that have not yet been indicated for treatment with antibodies that specifically bind to PD-1 and/or PD-L1 and/or CTLA-4, for example if it has been previously found that these antibodies (or combinations of antibodies) do not exert a measurable therapeutic effect.

We also include cancers that may have a low tumour mutational burden (TMB). By “tumour mutational burden” we mean the number of gene mutations within cancer cells. Such a measurement can be determined by laboratory tests known in the art. It is known that cells with a high TMB are more likely to be recognised as abnormal and attacked by the immune system, and high TMB has been identified as a response biomarker for PD-1/PD-L1 blockade (Goodman et al., 2017, Mol Cancer Ther., 16(11): 2598-2608). Therefore, cancers with a low TMB may be successfully targeted with the combination of the disclosure, which can enhance the effects of such immune blockade antibodies as described above. Multiple biomarkers have been associated with immune checkpoint inhibitor (“CPI”) response to date, which can be broadly grouped into the following categories: i) sources of antigen which elicit T cell response; ii) mechanisms of immune evasion which drive resistance; and iii) markers of immune infiltration. Additional factors that regulate response to immune checkpoint blockade and antibody-based cancer immunotherapy and, conversely, lack of response and resistance have been described and are continuously being identified. For example, a recent systematic pan-tumor analyses comprising collat whole-exome and transcriptomic data for >1000 CPI-treated patients across eight tumor-types, utilizing standardized bioinformatics-workflows and clinical outcome-criteria to validate multivariate predictors of CPI-sensitization identified Clonal-TMB as the strongest predictor of CPI response, followed by TMB and CXCL9 expression [23]. Discovery analysis identified two additional determinants of CPI-response supported by prior functional evidence: 9q34.3 (TRAF2) loss and CCND1 amplification, both of which were independently validated in >1600 CPI-treated patients. Further, scRNA sequencing of clonal neoantigen-reactive CD8-TILs, combined with bulk RNAseq analysis of CPI responding tumors, identified CCR5 and CXCL13 as T cell-intrinsic mediators of CPI-sensitisation.

A person skilled in the art will appreciate that new markers of likelihood of response, and conversely lack of response and resistance, are continuously being identified and may relate to e.g. sources of antigen which elicit T cell response, mechanisms of immune evasion which drive resistance, and markers of immune infiltration, for example as recently described and discussed in [23].

It will be appreciated that cancer that is resistant to treatment may be a relapsed and/or refractory cancer, in some embodiments.

A relapsed cancer is a cancer that has previously been treated and, as a result of that treatment, the subject made a complete or partial recovery (i.e. the subject is said to be in remission), but that after the cessation of the treatment the cancer returned or worsened. Put another way, a relapsed cancer is one that has become resistant to a treatment, after a period in which it was effective and the subject made a complete or partial recovery.

A refractory cancer is a cancer that has been treated but which has not responded to that treatment, and/or has been treated but which has progressed during treatment. Put another way, a refractory cancer is one that is resistant to a treatment.

It will be appreciated that a cancer may be a refractory cancer due to an intrinsic resistance. By “intrinsic resistance”, we include the meaning that the cancer and/or the subject and/or the target cell is resistant to a particular treatment from the first time at which it is administered, or before it is administered at all.

It will be appreciated that a cancer may be a relapsed cancer, or a relapsed cancer and a refractory cancer, due to an acquired resistance. By “acquired resistance”, we include that the cancer and/or the subject and/or the target cell was not resistant to a particular treatment prior to the first time it was administered, but became resistant after or during at least the first time it was administered—for example: after the second time; after the third time; after the fourth time; after the fifth time; after the sixth time; after the seventh time; after the eighth time; after the ninth time; after the tenth time; after the eleventh time; after the twelfth time the treatment was administered.

A relapsed cancer and/or refractory cancer would be readily diagnosed by one skilled in the art of medicine.

The present disclosure may be particularly useful in treating cancers that are not typically well targeted by the immune system (which are also known in the art as “cold tumours”). Such cold tumours can be classified into the following types: immune deserted tumours, i.e. there is a total lack of immune response in the tumour due to a lack of tumor-infiltrating T cells, immune excluded tumours, i.e. responsive T cells are generated but are unable to penetrate the tumour to mount a response against it, T cells may be present at the tumour periphery, and tumours with a poor immune infiltration, i.e. the level of penetration of immune cells (T cells) into the tumour microenvironment is reduced or eliminated.

Methods for identifying and/or classifying such tumours will be known to those skilled in the art. For example, immunohistochemistry may be used to detect presence or absence of CD8+ T cells in a tumour, and such approaches are used (albeit with different cut-offs and reagents) to generate “immunoscores”.

Cancers that may fall into these “cold tumour” subtypes include, but are not limited, to the following: melanoma, pancreatic cancer, prostate cancer, colorectal cancer, hepatocellular carcinoma, lung cancer, bladder cancer, kidney cancer, gastric cancer, cervical cancer, Merkel cell carcinoma, or ovarian cancer.

Therefore, the present disclosure may be particularly useful in combating resistance to anti-CTLA-4, anti-PD1 and/or anti-PD-L1 therapies in patients with these types of tumours, through simultaneous blockade of FcγRIIb and enhancement of immune effector cell activation which in turn enhances the therapeutic effect of the second and/or third antibody molecules.

In some embodiments, the patient that is resistant to treatment with an antibody molecule that specifically binds to PD-1 or PD-L1, and/or an antibody molecule that specifically binds to CTLA-4 has previously been treated with an antibody molecule that specifically binds to PD-1 or PD-L1, and/or an antibody molecule that specifically binds to CTLA-4, optionally wherein the patient has become resistant following said treatment.

In this embodiment, we include the situation where the patient has previously been treated with a first antibody that specifically binds to PD-1, and the second antibody molecule of the present disclosure is a second antibody that specifically binds to PD-1 (i.e. the second antibody molecule of the present disclosure is different to the anti-PD-1 antibody previously used to treat the patient). In some alternative embodiments, the antibody that specifically binds to PD-1 that was previously used to treat the patient is the same as the second antibody molecule of the present disclosure that specifically binds PD-1.

In this embodiment, we also include the situation where the patient has previously been treated with a first antibody that specifically binds to PD-L1, and the second antibody molecule of the present disclosure is a second antibody that specifically binds to PD-L1 (i.e. the second antibody molecule of the present disclosure is different to the anti-PD-L1 antibody previously used to treat the patient). In some alternative embodiments, the antibody that specifically binds to PD-L1 that was previously used to treat the patient is the same as the second antibody molecule of the present disclosure that specifically binds PD-L1.

In this embodiment, we also include the situation where the patient has previously been treated with a first antibody that specifically binds to CTLA-4, and the third antibody molecule of the present disclosure is a third antibody that specifically binds to CTLA-4 (i.e. the third antibody molecule of the present disclosure is different to the anti-CTLA-4 antibody previously used to treat the patient). In some alternative embodiments, the antibody that specifically binds to CTLA-4 that was previously used to treat the patient is the same as the third antibody molecule of the present disclosure that specifically binds CTLA-4.

As discussed above, in some alternative embodiments, the patient has not previously been treated with an antibody molecule that specifically binds to PD-1 or PD-L1, and/or an antibody molecule that specifically binds to CTLA-4. In this embodiment, the patient may be inherently resistant to said treatment.

In some embodiments, patients that may benefit from treatment with the combination of the first, second and third antibody molecules defined herein may be identified using an immunoscore test, which determines whether the tumour is positive or negative for certain target antigens, in this case CTLA-4 and/or PD-1 and/or PD-L1. Such a determination may be made by histological staining for the antigen in question, and a sample is described as positive if the percentage of cells expressing that antigen (by either total or partial staining) is above a pre-determined cut-off value. Such scoring is termed a Tumour Proportion Score (TPS). The TPS can be used to predict if a patient will be responsive to a monoclonal antibody therapy targeting that antigen.

For example, in the case of antibody molecules targeting PD-1 or PD-L1, it has been established that a sample is considered PD-L1 positive if the TPS is determined as 50% or greater (for viable tumour cells exhibiting membrane staining at any intensity). See, for example, the FDA approved test at: https://www.accessdata.fda.gov/cdrh_does/pdf15/P150013B.pdf.

Therefore, in order to determine which patients may benefit from the treatment with the combination of the first, second and third antibody molecules defined herein, it may be advantageous to identify patients who are determined as PD-L1 negative using the above mentioned test, i.e. that have from 1% to less than 50% of viable tumour cells staining for PD-L1. This patient group is unlikely to be responsive to therapy with anti-PD-1 or anti-PD-L1 antibodies alone (or in combination with antibodies targeting CTLA-4), but is more likely to be responsive to the combination therapy described herein for the reasons described above. Therefore, in some embodiments, the patients as defined herein are defined as PD-L1 negative using an established diagnostic test or IHC methods. Such tests may include an immunoscore test (as are known in the art and are discussed herein) to detect and evaluate the percentage of immune cells and/or tumour cells that are positive for a particular marker, such as PD-L1. The skilled person will understand that similar tests can be carried out to determine the CTLA-4 status of a patient. Further, using similar methodology, tumors can be analysed for T cell and additional tumor-infiltrating lymphocytes status, indicating whether the tumor is of “hot” T cell inflamed or “cold” Immune excluded or immune desert phenotypes—indicating whether a particular patient is likely to be resistant to ant-PD-1/L1 and/or anti-CTLA-4 immune checkpoint blockade, yet be responsive to herein disclosed combination treatment(s).

In some additional or alternative embodiments, patients that may benefit from treatment with the combination of the first, second and third antibody molecules defined herein may be identified by an immunohistochemical analysis to determine if the number of immune cells infiltrating the tumour is reduced. By “reduced”, we mean that the number of infiltrating immune cells (e.g. T cells) in the tumour is lower than expected for normal tumours where immune infiltration is observed.

The use of such a test will be particularly advantageous in the situation where a patient has never been treated with antibodies that are specific for PD-1, PD-L1 or CTLA-4 before. In this case, the standard tests described above would classify many patients as not being responsive to these treatments, in which case they may not be used. The present disclosure can widen the potential use of these treatments to patient groups that were previously thought not to be responsive.

In some embodiments the first antibody molecule that specifically binds FcγRIIb and the second antibody molecule and/or the third antibody molecule are administered simultaneously to the patient, meaning that they are either administered together at one or separately very close in time to each other.

In some embodiments the antibody molecule that specifically binds FcγRIIb is administered to the patient prior to administration of the second antibody molecule. In some embodiments the antibody molecule that specifically binds FcγRIIb is administered to the patient prior to administration of the third antibody molecule.

Such sequential administration may be achieved by temporal separation of the antibodies. Alternatively, or in combination with the first option, the sequential administration may also be achieved by spatial separation of the antibody molecules, by administration of the antibody molecule that specifically binds FcγRIIb in a way, such as intratumoural, so that it reaches the cancer prior to the second and/or third antibody molecule, which is then administered in a way, such as systemically, so that it reaches the cancer after the antibody molecule that specifically binds FcγRIIb.

In some embodiments the second antibody molecule is administered to the patient prior to administration of the antibody molecule that specifically binds FcγRIIb. In some embodiments the third antibody molecule is administered to the patient prior to administration of the antibody molecule that specifically binds FcγRIIb.

It would be known to the person skilled in medicine, that medicines can be modified with different additives, for example to change the rate in which the medicine is absorbed by the body; and can be modified in different forms, for example to allow for a particular administration route to the body.

Accordingly, we include that the antibodies and compositions described herein may be combined with an excipient and/or a pharmaceutically acceptable carrier and/or a pharmaceutically acceptable diluent and/or an adjuvant.

We also include that the combination, and/or composition, and/or antibody, and/or medicament of the disclosure may be suitable for parenteral administration including aqueous and/or non-aqueous sterile injection solutions which may contain anti-oxidants, and/or buffers, and/or bacteriostats, and/or solutes which render the formulation isotonic with the blood of the intended recipient; and/or aqueous and/or non-aqueous sterile suspensions which may include suspending agents and/or thickening agents. The combination, and/or composition, and/or antibody, and/or agent, and/or medicament of the disclosure may be presented in unit-dose or multi-dose containers, for example sealed ampoules and vials, and may be stored in a freeze-dried (i.e. lyophilised) condition requiring only the addition of the sterile liquid carrier, for example water for injections, immediately prior to use.

Extemporaneous injection solutions and suspensions may be prepared from sterile powders, and/or granules, and/or tablets of the kind previously described.

For parenteral administration to human patients, the daily dosage level of the antibody molecule that specifically binds FcγRIIb and/or the second antibody molecule and/or the third antibody molecule will usually be from 1 mg/kg bodyweight of the patient to 20 mg/kg, or in some cases even up to 100 mg/kg administered in single or divided doses. In some embodiments, the dose of the antibody molecules is 10 mg/kg, 3 mg/kg or 1 mg/kg. Lower doses may be used in special circumstances, for example in combination with prolonged administration. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this disclosure.

Typically, the composition and/or medicament of the disclosure will contain the antibody molecule that specifically binds FcγRIIb and/or the second/third antibody at a concentration of between approximately 2 mg/ml and 150 mg/ml or between approximately 2 mg/ml and 200 mg/ml. In a preferred embodiment, the medicaments and/or compositions of the disclosure will contain the antibody molecule that specifically binds FcγRIIb and/or the second/third antibody molecule at a concentration of 10 mg/ml.

Generally, in humans, oral or parenteral administration of the composition, and/or antibody, and/or agent, and/or medicament of the disclosure is the preferred route, being the most convenient. For veterinary use, the composition, and/or antibody, and/or agent and/or medicament of the disclosure are administered as a suitably acceptable formulation in accordance with normal veterinary practice and the veterinary surgeon will determine the dosing regimen and route of administration which will be most appropriate for a particular animal. Thus, the present disclosure provides a pharmaceutical formulation comprising an amount of an antibody and/or agent of the disclosure effective to treat various conditions (as described above and further below). Preferably, the composition, and/or antibody, and/or agent, and/or medicament is adapted for delivery by a route selected from the group comprising: intravenous (IV); subcutaneous (SC), intramuscular (IM), or intratumoural. In some preferred embodiments, the administration is intravenous.

In some embodiments, the first antibody molecule and/or the second antibody and/or the third antibody molecule may be administered through the use of plasmids or viruses. Such plasmids then comprise nucleotide sequences encoding either the first antibody molecule and/or the second antibody and/or the third antibody molecule. In some embodiments, nucleotide sequences encoding parts of or the full sequences of the first antibody molecule and/or the second antibody and/or the third antibody molecule are integrated in a cell or viral genome or in a viriome in a virus; such a cell or virus then act as a delivery vehicle for the first antibody molecule and/or the second antibody and/or the third antibody molecule (or a delivery vehicle for a nucleotide sequence encoding the first antibody molecule and/or the second antibody and/or the third antibody molecule). For example, in some embodiments, such a virus may be in the form of a therapeutic oncolytic virus comprising nucleotide sequences encoding at least one of the antibody molecules described herein. In some embodiments, such an oncolytic virus comprises nucleotide sequences encoding a full-length human IgG antibody. Oncolytic viruses are known to those skilled in the arts of medicine and virology.

The present disclosure also includes the composition, and/or antibody, and/or agent, and/or medicament comprising pharmaceutically acceptable acid or base addition salts of the polypeptide binding moieties. The acids which are used to prepare the pharmaceutically acceptable acid addition salts of the aforementioned base compounds useful in this disclosure are those which form non-toxic acid addition salts, i.e. salts containing pharmacologically acceptable anions, such as the hydrochloride, hydrobromide, hydroiodide, nitrate, sulphate, bisulphate, phosphate, acid phosphate, acetate, lactate, citrate, acid citrate, tartrate, bitartrate, succinate, maleate, fumarate, gluconate, saccharate, benzoate, methanesulphonate, ethanesulphonate, benzenesulphonate, p-toluenesulphonate and pamoate [i.e. 1,1′-methylene-bis-(2-hydroxy-3 naphthoate)] salts, among others. Pharmaceutically acceptable base addition salts may also be used to produce pharmaceutically acceptable salt forms of the agents according to the present disclosure. The chemical bases that may be used as reagents to prepare pharmaceutically acceptable base salts of the present agents that are acidic in nature are those that form non-toxic base salts with such compounds. Such non-toxic base salts include, but are not limited to those derived from such pharmacologically acceptable cations such as alkali metal cations (e.g. potassium and sodium) and alkaline earth metal cations (e.g. calcium and magnesium), ammonium or water-soluble amine addition salts such as N-methylglucamine-(meglumine), and the lower alkanolammonium and other base salts of pharmaceutically acceptable organic amines, among others. The agents and/or polypeptide binding moieties of the disclosure may be lyophilised for storage and reconstituted in a suitable carrier prior to use. Any suitable lyophilisation method (e.g. spray drying, cake drying) and/or reconstitution techniques can be employed. It will be appreciated by those skilled in the art that lyophilisation and reconstitution can lead to varying degrees of antibody activity loss (e.g. with conventional immunoglobulins, IgM antibodies tend to have greater activity loss than IgG antibodies) and that use levels may have to be adjusted upward to compensate. In one embodiment, the lyophilised (freeze dried) polypeptide binding moiety loses no more than about 20%, or no more than about 25%, or no more than about 30%, or no more than about 35%, or no more than about 40%, or no more than about 45%, or no more than about 50% of its activity (prior to lyophilisation) when re-hydrated.

The disclosure also relates generally to a combination comprising a first and a second antibody, which is described herein in the eighth to fourteenth aspects of the disclosure:

In a further eighth aspect, the disclosure provides for use of a first antibody molecule that specifically binds to FcγRIIb via its Fab region, and that lacks an Fc region or has reduced binding to Fcγ receptors via its Fc region, for treating cancer in a patient;

characterised in that the first antibody molecule reduces and/or prevents resistance in the cancer to treatment with a second antibody molecule that specifically binds to CTLA-4. In one particular embodiment of this aspect, the dose of the antibody molecule that specifically binds to CTLA-4 is lower than the therapeutic dose.

In a ninth aspect, the disclosure also provides a combination comprising a first antibody molecule that specifically binds to FcγRIIb via its Fab region, and that lacks an Fc region or has reduced binding to Fcγ receptors via its Fc region; and a second antibody molecule that specifically binds to CTLA-4 and that binds to at least one Fcγ receptor via its Fc region, for use in treating cancer in a patient, wherein the combination comprises a dose of the second antibody molecule that is lower than the tolerated therapeutic dose.

In a tenth aspect, the disclosure provides use of a first antibody molecule that specifically binds to FcγRIIb via its Fab region, and that lacks an Fc region or has reduced binding to Fcγ receptors via its Fc region; and a second antibody molecule that specifically binds to CTLA-4 and that binds to at least one Fcγ receptor via its Fc region, in the manufacture of a medicament for treating cancer in a patient, wherein the combination comprises a dose of the second antibody molecule that is lower than the tolerated therapeutic dose.

In an eleventh aspect, the disclosure provides a method for treating cancer in an individual, the method comprising administering to the patient (i) a first antibody molecule that specifically binds to FcγRIIb via its Fab region, and that lacks an Fc region or has reduced binding to Fcγ receptors via its Fc region, and (ii) a second antibody molecule that specifically binds to CTLA-4 and that binds to at least one Fcγ receptor via its Fc region, wherein the dose of the second antibody molecule that is administered is lower than the tolerated therapeutic dose.

In a twelfth aspect, the disclosure provides a first antibody that specifically binds to FcγRIIb via its Fab region, and that lacks an Fc region or has reduced binding to Fcγ receptors via its Fc region, for use in combination with a second antibody molecule that specifically binds to CTLA-4 and that binds to at least one Fcγ receptor via its Fc region, for treating cancer in a patient, wherein the dose of the second antibody molecule that is used is lower than the tolerated therapeutic dose.

In a thirteenth aspect, the disclosure provides a pharmaceutical composition comprising a first antibody molecule that specifically binds to FcγRIIb via its Fab region, and that lacks an Fc region or has reduced binding to Fcγ receptors via its Fc region, and a second antibody molecule that specifically binds to CTLA-4 and that binds to at least one Fcγ receptor via its Fc region, wherein the second antibody molecule is present at a dose which is lower than the tolerated therapeutic dose.

In a fourteenth aspect, the disclosure provides a kit comprising a first antibody molecule that specifically binds to FcγRIIb via its Fab region, and that lacks an Fc region or has reduced binding to Fcγ receptors via its Fc region, and a second antibody molecule that specifically binds to CTLA-4 and that binds to at least one Fcγ receptor via its Fc region, wherein the second antibody molecule is present at a dose that is lower than the tolerated therapeutic dose.

As discussed above, the dose of the second antibody in accordance with the above aspects is or may be lower than the tolerated therapeutic dose.

Thus, it will be appreciated that the eighth to fourteenth aspects of the disclosure are based on the inventors' surprising discovery that, upon combination of the first and the second antibody molecule, the second antibody molecule can be used at a lower, better tolerated, dose with retained or greater therapeutic efficacy compared to when the second antibody is used alone at (the same or) higher doses.

By “tolerated therapeutic dose” we mean any dose that would be considered to be therapeutically active (i.e. produces the desired therapeutic effect in a patient or subject defined herein), but that is considered to be tolerated (i.e. does not produce unacceptable levels of toxicity or side effects in the patient). The skilled person will appreciate that the dose that is chosen is often a compromise between achieving a therapeutic effect, and not causing unacceptable toxicity to the patient.

By “therapeutically active” we include where the dose produces the desired therapeutic effect in a patient or subject. In the case of the second antibody molecule that specifically binds to CTLA-4 and that binds to at least one Fcγ receptor via its Fc region, such a therapeutic effect may be a reduction in tumour volume in the patient.

By “therapeutic effect” we include all effects that are attributable directly or indirectly to use of the therapy in question. This may be a measurable therapeutic effect, such as reduced tumour volume or reduced tumour size (which may be determined by a CT scan, for example). In other cases, this may be a more subjective effect, such as a reduction in severity of symptoms reported by the patient. The measurement of therapeutic effects in cancer patients in response to the administration of therapeutic antibodies is well known in the art. Furthermore, the level of survival of a patient or group of patients over a defined time period is an alternative read-out of therapeutic effect.

In some cases, the dose that is lower than the tolerated therapeutic dose is lower than the recommended tolerated therapeutic dose. The skilled person will be aware that for approved antibody therapies, certain doses (typically expressed in mg/kg) are recommended for use in certain patient groups or for patients with a particular type of cancer. Often recommended therapeutic doses are described in the labelling or prescription information of an approved antibody therapeutic. Otherwise, it would be apparent to the skilled person how to determine the recommended tolerated therapeutic dose using techniques well known in the art.

In some other cases, the dose that is lower than the tolerated therapeutic dose is lower than the calculated therapeutic dose. By “calculated therapeutic dose” we include the dose of the antibody that has been calculated for a particular patient, i.e. based on the type of cancer, the stage of the cancer, their weight, Body Mass Index (BMI) and other factors.

In some other cases, the dose that is lower than the tolerated therapeutic dose is lower than the maximum (or the maximum approved) tolerated therapeutic dose. By maximum tolerated therapeutic dose, we mean the highest dose that does not cause unacceptable side effects.

In some other cases, the dose that is lower than the tolerated therapeutic dose is lower than the minimum therapeutic dose (otherwise known as the minimum effective dose). By the minimum therapeutic dose, we mean the lowest dose that would be considered to generate a measurable therapeutic effect in a patient, as defined above.

In some other cases, the dose that is lower than the tolerated therapeutic dose is lower than the recommended tolerated therapeutic dose. In some embodiments, this may include the recommended dose for the indication included in the drug label.

The skilled person will appreciate that the actual dosage values of the tolerated therapeutic doses defined above will differ depending on the identity of the antibody that specifically binds to CTLA-4, and the patient in which the combination is used, or is suitable for use in.

It will be appreciated that the recommended dose is the dose approved by a regulatory agency such as the FDA or EMEA. This dose is typically identified following review of both efficacy and tolerability data often from late phase placebo-controlled blinded and randomized clinical trials, which may include different dose-levels. In cancer, approved doses will have therapeutic benefit and show acceptable toxicity. Through development (from early to later stage clinical trials) it is sometimes found that higher antibody doses are more efficacious but also associated with unacceptable toxicity.

It will be apparent to the skilled person in the art how the particular tolerated therapeutic dose is defined for any particular antibody, generally using dose escalation studies during clinical trials. For instance, the tolerated therapeutic doses of an example antibody that binds specifically to CTLA-4, ipilimumab, are set out in the drug label (see the FDA label for ipilimumab at: https://www.accessdata.fda.gov/drugsatfda_docs/label/2018/125277s094lb1.pdf).

As discussed therein, the tolerated therapeutic dose of ipilimumab may be as follows. For unresectable or metastatic melanoma: 3 mg/kg administered intravenously over 90 minutes every 3 weeks for a total of 4 doses. For adjuvant melanoma: 10 mg/kg administered intravenously over 90 minutes every 3 weeks for 4 doses, followed by 10 mg/kg every 12 weeks for up to 3 years or until documented disease recurrence or unacceptable toxicity. For advanced renal cell carcinoma: Nivolumab 3 mg/kg administered intravenously over 30 minutes followed by ipilimumab 1 mg/kg administered intravenously over 30 minutes on the same day, every 3 weeks for a maximum of 4 doses, then nivolumab 240 mg every 2 weeks or 480 mg every 4 weeks, administered intravenously over 30 minutes.

Therefore, the tolerated therapeutic dose of ipilimumab may be 1 mg/kg, 3 mg/kg or 10 mg/kg, in some embodiments.

Tolerated therapeutic doses for antibodies that have not yet been approved may be based on the tolerated therapeutic doses of similar antibodies that have been approved or have undergone extensive clinical testing.

By providing the combination of the first and second antibody described in the preceding aspects wherein the second antibody is used in a dose lower than the tolerated therapeutic dose, the inventors have devised a way of achieving a similar or comparable therapeutic effect to when much higher doses of the same antibody targeting CTLA-4 is used in isolation.

Using high doses of antibodies targeting CTLA-4 is generally undesirable from a tolerability perspective. CTLA-4 is expressed by activated T cells and transmits an inhibitory signal to T cells, thereby downregulating the T cell response. Blocking CTLA-4 using a therapeutic antibody that binds specifically to CTLA-4 prevents this inhibitory signal, thereby activating more T cells that can target the cancer. However, this mechanism is indiscriminate and can activate more T cells that target self-specific antigens not found on tumour cells, i.e. it can initiate an autoimmune response. Therefore, by reducing the dose of anti-CTLA-4 antibody required to produce a therapeutic effect, it is possible to reduce problems related to tolerability.

Using a lower dose and achieving the same therapeutic effect can be achieve using a combination with the first antibody molecule that specifically binds to FcγRIIb via its Fab region, and that lacks an Fc region or has reduced binding to Fcγ receptors via its Fc region.

The first antibody molecule blocks binding to the inhibitory FcγRIIb, which can in turn activate effector immune cells that can target cancer cells, e.g. CD8 effector T cells.

This is surprising as, especially in patients who are resistant to or who have developed resistance to or may develop resistance to anti-CTLA-4 treatment, it would not be expected that reducing the dose of anti-CTLA-4 antibody would lead to a comparable therapeutic effect as using a much higher dose of the same antibody.

Using a lower dose of the antibody molecule that specifically binds CTLA-4 is therefore advantageous, as by using lower doses, there is a reduced chance of the patient having issues related to tolerability (i.e. tolerability is improved), toxicity and unpleasant side effects. It also improves the cost-effectiveness of the treatment, as less antibody is required for administration.

In some embodiments, the continued use of lower doses of antibodies targeting CTLA-4 could reduce the risk of subjects becoming resistant. Without being bound by theory, the inventors believe that, for example, if intratumoral Treg depletion is better achieved and a therapeutically more efficacious mechanism of action occurs at low(er) anti-CTLA-4 doses, then low level blockade of inhibitory signalling in effector T cells combined with enhanced Treg depletion (using the combination of first and second antibody molecules described herein) could reduce and/or prevent resistance.

Therefore, the present disclosure makes it possible to extend the therapeutic window of antibodies that are specific to CTLA-4. By “therapeutic window” we mean the range of drug doses that can effectively treat a disease without having toxic effects or tolerability problems. Therefore, the present disclosure makes it possible to use lower doses of anti-CTLA-4 antibodies and achieve the same or similar therapeutic effects while lowering the possibility of adverse effects due to the lower doses.

In some embodiments, the dose of the second antibody molecule can be expressed as a percentage of the tolerated therapeutic dose as defined herein. In some embodiments, the dose of the second antibody is at least 30%, 35%, 40%, 45%, 50%, 55%, 60%, 65%, 70%, 75%, 80%, 85% or 90% lower than the tolerated therapeutic dose of the second antibody molecule. In some preferred embodiments, the dose of the second antibody is at least 50% lower than the tolerated therapeutic dose of the second antibody. In some other preferred embodiments, the dose of the second antibody is at least 70% lower than the tolerated therapeutic dose of the second antibody.

In some preferred embodiments, the dose of the second antibody is at least 80% lower (i.e. 80% or lower) than the tolerated therapeutic dose of the second antibody.

As discussed above, in some embodiments, the result of using the second antibody molecule at a dose that is lower than the tolerated therapeutic dose is that the therapeutic effect of the first antibody molecule and the second antibody molecule used at the lower dose is comparable to the therapeutic effect of the second antibody molecule in the absence of the first antibody molecule at the maximum tolerated therapeutic dose of the second antibody molecule. This effect would be readily measured by a person skilled in the art, as discussed above in relation to the meaning of the therapeutic effect.

As also discussed above, the use of the second antibody molecule at doses that are lower than the tolerated therapeutic dose may, in some embodiments, improve the tolerability of the second antibody molecule in the subject.

The term “tolerability” as used herein refers to the degree to which adverse effects of a therapeutic agent can be tolerated by a subject. By “adverse effect” we include any effect caused by the therapeutic agent, either directly or indirectly, that is not the desired therapeutic effect, or any other beneficial effect attributable to the therapeutic agent, either directly or indirectly.

In the context of the second antibody that binds specifically to CTLA-4, these adverse effects may include one or more of the following: infusion related reactions (IRRs), fatigue, diarrhoea, enterocolitis, nausea, vomiting, pruritus, rash, colitis, cough, headache, unintended weight loss, decreased appetite, insomnia, pyrexia, hepatitis, dermatitis, immune-mediated neuropathies, and immune-mediated endocrinopathies.

Tolerability issues may be of different grades, i.e. of different severity for the patients experiencing them. In some cases, they lead to discomfort for the patient, while in others they may cause severe problems that may prevent continued treatment with the therapeutic antibody molecule. In severe cases toxicities may manifest as intestinal problems (colitis) that can cause tears or holes (perforation) in the intestines, liver problems (hepatitis) that can lead to liver failure, skin problems that can lead to severe skin reaction, nerve problems that can lead to paralysis, hormone gland problems (especially the pituitary, adrenal, and thyroid glands), lung problems (pneumonitis), kidney problems, including nephritis and kidney failure, and/or inflammation of the brain (encephalitis). In the worst of cases, the tolerability issues may even lead to death of the patient.

It will be appreciated that the most severe types of tolerability issues are not acute but rather take time (days to weeks) to manifest (consistent with the immune system requiring up to two weeks to mount certain T cell mediated immune responses) and include gastrointestinal perforation.

The tolerability issues that may be improved as described herein are adverse events that may occur in connection with intravenous administration of the therapeutic antibody molecule to a subject.

In some embodiments, using a dose of the second antibody that is lower than the tolerated therapeutic dose reduces side effects and/or reduces toxicity in the subject associated with the use of the second antibody molecule.

By “side-effects” we include any of the “adverse effects” discussed above in relation to tolerability that has been caused by the therapeutic agent. It is known that reducing the dose of therapeutic antibodies reduces the associated side effects, however it is also known that this reduces the therapeutic effect (for ipilimumab, see Wolchok et al., 2010, Lancet Oncol., 11(2):155-164).

By “toxicity” we mean the degree to which a therapeutic substance can damage an organism. The skilled person will understand that toxicity and tolerability are interrelated and are both dependent on the dose administered.

In the case of therapeutic antibodies as discussed herein, toxicity may occur when large amounts of the therapeutic antibody build up in the body. It is therefore advantageous to administer lower doses of antibody therapeutics to minimise any issues relating to toxicity.

In some other embodiments, using a dose of the second antibody that is lower than the tolerated therapeutic dose may reduce any off-target effects and/or autoimmune reactions in the subject associated with the use of the second antibody molecule.

As described herein, the second antibody molecule specifically binds to CTLA-4 and binds to at least one Fcγ receptor via its Fc region. CTLA-4 is constitutively expressed in regulatory T cells but only upregulated in conventional T cells after activation—a phenomenon which is particularly notable in cancers. In some embodiments, the second antibody molecule is ipilimumab (such as Yervoy® from Bristol-Myers Squibb). In some embodiments the second antibody molecule is tremelimumab (formerly denoted ticilimumab and, CP-675,206), which is a fully human monoclonal antibody against CTLA-4, previously in development by Pfizer and now in clinical development by MedImmune.

Therefore, in some embodiments, the second antibody molecule is ipilimumab. The skilled person will understand that the standard tolerated therapeutic doses of ipilimumab can be determined from the approved drug labelling. In some embodiments, when the second antibody molecule is ipilimumab, the tolerated therapeutic dose is 10 mg/kg. Therefore, the dose of the second antibody that is lower than the tolerated therapeutic dose is any dose lower than 10 mg/kg. In this case, the treatment may be adjuvant therapy, i.e. for treatment of cancer that has already been treated with one or more primary treatments, e.g. surgery.

In some embodiments, when the second antibody molecule is ipilimumab, the tolerated therapeutic dose is 3 mg/kg. Therefore, the dose of the second antibody that is lower than the tolerated therapeutic dose is any dose lower than 3 mg/kg. For instance, in some preferred embodiments, the dose of the second antibody may be about 2 mg/kg or may be lower than 2 mg/kg, e.g. in the range of 1.5 mg/kg to 2.5 mg/kg. In some embodiments, the dose of the second antibody is 2 mg/kg. In some embodiments, the dose of the second antibody is 1 mg/kg, when the second antibody is ipilimumab.

In some embodiments, the tolerated therapeutic dose of the second antibody molecule when the second antibody molecule is ipilimumab is 1 mg/kg. Therefore, in this embodiment, the dose of the second antibody that is lower than the tolerated therapeutic dose is any dose lower than 1 mg/kg. In this embodiment, ipilimumab is approved for use in combination with another therapeutic antibody e.g. Nivolumab.

Therefore, in some embodiments, the use or method as described in relation to the eight to fourteenth aspects does not also involve administration of an antibody molecule that specifically binds PD-1 or PD-L1 and/or the pharmaceutical composition or kit does not also comprise an antibody molecule that specifically binds PD-1 or PD-L1. In these embodiments, the tolerated therapeutic dose of ipilimumab is typically 3 mg/kg or higher.

In the embodiments where the second antibody molecule is ipilimumab, the second antibody molecule may be administered in accordance with a dosage schedule as provided in the approved label, or alternatively a different dosage schedule may be possible using the lower doses contemplated herein.

In some additional or alternative embodiments, the second antibody molecule is tremelimumab.

In some embodiments, when the second antibody molecule is tremelimumab, the tolerated therapeutic dose is 750 mg. Therefore, the dose of the second antibody that is lower than the tolerated therapeutic dose is any dose lower than 750 mg. In some other embodiments, the tolerated therapeutic dose is 300 mg. Therefore, the dose of the second antibody that is lower than the tolerated therapeutic dose is any dose lower than 300 mg. In some other embodiments, the tolerated therapeutic dose is 75 mg. Therefore, the dose of the second antibody that is lower than the tolerated therapeutic dose is any dose lower than 75 mg.

In some embodiments, when the second antibody molecule is tremelimumab, the tolerated therapeutic dose is 10 mg/kg. Therefore, the dose of the second antibody that is lower than the tolerated therapeutic dose is any dose lower than 10 mg/kg.

In some embodiments, when the second antibody molecule is tremelimumab, the tolerated therapeutic dose is 3 mg/kg. Therefore, the dose of the second antibody that is lower than the tolerated therapeutic dose is any dose lower than 3 mg/kg. For instance, in some preferred embodiments, the dose of the second antibody may be about 2 mg/kg or may be lower than 2 mg/kg, e.g. in the range of 1.5 mg/kg to 2.5 mg/kg. In some embodiments, the dose of the second antibody is 2 mg/kg. In some embodiments, the dose of the second antibody is 1 mg/kg, when the second antibody is tremelimumab.

In some embodiments, the tolerated therapeutic dose of the second antibody molecule when the second antibody molecule is tremelimumab is 1 mg/kg. Therefore, in this embodiment, the dose of the second antibody that is lower than the tolerated therapeutic dose is any dose lower than 1 mg/kg.

In the embodiments where the second antibody molecule is tremelimumab, the second antibody molecule may be administered in accordance with a dosage schedule as provided in the approved label or approved clinical trial schedule, or alternatively a different dosage schedule may be possible using the lower doses contemplated herein.

The skilled person will appreciate that the second antibody molecule may be a combination of any of the antibodies specific for CTLA-4 discussed herein, for example, the second antibody molecule that specifically binds to CTLA-4 may be a combination of ipilimumab and tremelimumab.

It will also be appreciated that other antibodies that are specific for CTLA-4 are also contemplated by the disclosure, aside from those discussed specifically.

As discussed above, the second antibody molecule may reduce and/or prevent resistance in the cancer to treatment with a second antibody molecule that specifically binds to CTLA-4.

In some embodiments, the disclosure described in the eighth to fourteenth embodiments is for use in treating subject who have a cancer that is resistant to treatment. In some embodiments, the cancer may be relapsed or refractory cancer. In some embodiments, the cancer may be resistant to treatment with antibodies that target the immune checkpoint blockade, for example, antibodies that are specific for CTLA-4.

By “resistant”, “resistance”, or “resistant to treatment” we mean that the patient has a reduced level of responsiveness to treatment with an antibody molecule that specifically binds to CTLA-4, compared to a previous level of responsiveness or expected level of responsiveness. This includes the situation where a patient has previously been treated with said antibody molecules (i.e. they have acquired resistance), and also includes the situation where the patient has never been treated with said antibody molecules (i.e. they are inherently resistant). In some additional or alternative embodiments, the patient may have a reduced level of responsiveness to antibody molecules that specifically bind to PD-1 and/or PD-L1.

Resistance to treatment can be measured in a variety of ways, for instance, by monitoring the patient to ensure that the cancer is receding in the expected way, and identifying patients not responding at all to the treatment.

By “resistant to treatment” we also include types of cancer that have not yet been indicated for treatment with antibodies that specifically bind to CTLA-4, for example if it has been previously found that these antibodies (or combinations of antibodies) do not exert a measurable therapeutic effect.

We also include cancers that may have a low tumour mutational burden (TMB). By “tumour mutational burden” we mean the number of gene mutations within cancer cells. Such a measurement can be determined by laboratory tests known in the art. It is known that cells with a high TMB are more likely to be recognised as abnormal and attacked by the immune system, and high TMB has been identified as a response biomarker for PD-1/PD-L1 blockade (Goodman et al., 2017, Mol Cancer Ther., 16(11): 2598-2608). Therefore, cancers with a low TMB may be successfully targeted with the combination of the disclosure, which can enhance the effects of such immune blockade antibodies as described above.

It will be appreciated that cancer that is resistant to treatment may be a relapsed and/or refractory cancer, in some embodiments.

A relapsed cancer is a cancer that has previously been treated and, as a result of that treatment, the subject made a complete or partial recovery (i.e. the subject is said to be in remission), but that after the cessation of the treatment the cancer returned or worsened. Put another way, a relapsed cancer is one that has become resistant to a treatment, after a period in which it was effective and the subject made a complete or partial recovery.

A refractory cancer is a cancer that has been treated but which has not responded to that treatment, and/or has been treated but which has progressed during treatment. Put another way, a refractory cancer is one that is resistant to a treatment.

It will be appreciated that a cancer may be a refractory cancer due to an intrinsic resistance. By “intrinsic resistance”, we include the meaning that the cancer and/or the subject and/or the target cell is resistant to a particular treatment from the first time at which it is administered, or before it is administered at all.

It will be appreciated that a cancer may be a relapsed cancer, or a relapsed cancer and a refractory cancer, due to an acquired resistance. By “acquired resistance”, we include that the cancer and/or the subject and/or the target cell was not resistant to a particular treatment prior to the first time it was administered, but became resistant after or during at least the first time it was administered—for example: after the second time; after the third time; after the fourth time; after the fifth time; after the sixth time; after the seventh time; after the eighth time; after the ninth time; after the tenth time; after the eleventh time; after the twelfth time the treatment was administered.

A relapsed cancer and/or refractory cancer would be readily diagnosed by one skilled in the art of medicine.

In some embodiments, the patient that is resistant to treatment with an antibody molecule that specifically binds to CTLA-4 has previously been treated with an antibody molecule that specifically binds to CTLA-4, optionally wherein the patient has become resistant following said treatment.

In this embodiment, we also include the situation where the patient has previously been treated with a first antibody that specifically binds to CTLA-4, and the second antibody molecule of the present disclosure is a second antibody that specifically binds to CTLA-4 (i.e. the second antibody molecule of the present disclosure is different to the anti-CTLA-4 antibody previously used to treat the patient). In some alternative embodiments, the antibody that specifically binds to CTLA-4 that was previously used to treat the patient is the same as the second antibody molecule of the present disclosure that specifically binds CTLA-4.

As discussed above, in some alternative embodiments, the patient has not previously been treated with an antibody molecule that specifically binds to CTLA-4. In this embodiment, the patient may be inherently resistant to said treatment.

In some embodiments, the cancer is a FcγRIIb-positive B-cell cancer. By “FcγRIIb-positive cancer”, we any cancer that expresses FcγRIIB, albeit at different levels. Fc RIIB expression is most pronounced in chronic lymphocytic leukaemia and mantle cell lymphomas, moderately so in diffuse large B cell lymphoma and least pronounced in follicular lymphomas. However, in some cases subjects with cancers that generally express low levels of Fc RIIB (e.g. follicular lymphomas) may have very high levels of Fc RIIB expression. The expression level of Fc RIIB in different types of B cell cancer (and, in particular, those mentioned above) correlates with rate of internalization of the antibody molecule Rituximab. Therefore, the expression of Fc RIIB and the associated internalization of antibody molecules is believed to be a common mechanism that is shared by B cell cancers (Lim et al., 2011). The Fc RIIB-dependent initialization of an antibody molecule can be blocked by herein disclosed antibodies to Fc RIIB.

Accordingly, the combinations disclosed herein may be used in treating B cell cancers, and, in particular, relapsed mantle cell lymphoma and/or refractory mantle cell lymphoma, and/or relapsed follicular lymphoma and/or refractory follicular lymphoma, and/or relapsed diffuse large B cell lymphoma and/or refractory diffuse large B cell lymphoma.

In some other more preferred embodiments the cancer is a FcγRIIb negative cancer. By “FcγRIIb negative cancer” we include any cancer that does not present any FcγRIIb receptors. This can be tested using anti-FcγRIIB specific antibodies in a variety of methods including immunohistochemistry and flow cytometry such as indicated in Tutt et al, J Immunol, 2015, 195 (11) 5503-5516.

In some preferred embodiments, the cancer is selected from the group consisting of carcinomas, sarcomas, and lymphomas.

In some embodiments, the cancer is a carcinoma selected from the group consisting of adenocarcinoma, squamous cell carcinoma, adenosquamous carcinoma, anaplastic or undifferentiated carcinoma, large cell carcinoma and small cell carcinoma.

In some embodiments, the cancer is a sarcoma selected from the group consisting of osteosarcoma, chondrosarcoma, liposarcoma, and leiomyosarcoma.

FcγRIIb negative cancer may be selected from the group consisting of melanoma, breast cancer, ovarian cancer, prostate cancer, metastatic hormone-refractory prostate cancer, colorectal cancer, lung cancer, small cell lung carcinoma (NSCLC), small cell lung cancer (SCLC), non-small cell lung cancer, urothelial carcinoma, bladder cancer, kidney cancer, mesothelioma, Merkel cell carcinoma, head and neck cancer, and pancreatic cancer.

Each one of the above described cancers is well-known, and the symptoms and cancer diagnostic markers are well described, as are the therapeutic agents used to treat those cancers. Accordingly, the symptoms, cancer diagnostic markers, and therapeutic agents used to treat the above mentioned cancer types would be known to those skilled in medicine, as discussed above in relation to the first to seventh aspects of the disclosure

In some embodiments the first antibody molecule that specifically binds FcγRIIb and the second antibody molecule that specifically binds CTLA-4 are administered simultaneously to the patient, meaning that they are either administered together at one or separately very close in time to each other.

In some embodiments the antibody molecule that specifically binds FcγRIIb is administered to the patient prior to administration of the second antibody molecule that specifically binds CTLA-4.

Such sequential administration may be achieved by temporal separation of the antibodies. Alternatively, or in combination with the first option, the sequential administration may also be achieved by spatial separation of the antibody molecules, by administration of the antibody molecule that specifically binds FcγRIIb in a way, such as intratumoural, so that it reaches the cancer prior to the second antibody molecule, which is then administered in a way, such as systemically, so that it reaches the cancer after the antibody molecule that specifically binds FcγRIIb.

In some embodiments the second antibody molecule that specifically binds CTLA-4 is administered to the patient prior to administration of the antibody molecule that specifically binds FcγRIIb, for example, using the spatial or temporal modes described above.

It would be known to the person skilled in medicine, that medicines can be modified with different additives, for example to change the rate in which the medicine is absorbed by the body; and can be modified in different forms, for example to allow for a particular administration route to the body.

Accordingly, we include that the antibodies and compositions described herein may be combined with an excipient and/or a pharmaceutically acceptable carrier and/or a pharmaceutically acceptable diluent and/or an adjuvant.

We also include that the combination, and/or composition, and/or antibody, and/or medicament of the disclosure may be suitable for parenteral administration including aqueous and/or non-aqueous sterile injection solutions which may contain anti-oxidants, and/or buffers, and/or bacteriostats, and/or solutes which render the formulation isotonic with the blood of the intended recipient; and/or aqueous and/or non-aqueous sterile suspensions which may include suspending agents and/or thickening agents. The composition, and/or antibody, and/or agent, and/or medicament of the disclosure may be presented in unit-dose or multi-dose containers, for example sealed ampoules and vials, and may be stored in a freeze-dried (i.e. lyophilised) condition requiring only the addition of the sterile liquid carrier, for example water for injections, immediately prior to use.

Extemporaneous injection solutions and suspensions may be prepared from sterile powders, and/or granules, and/or tablets of the kind previously described.

For parenteral administration to human patients, the daily dosage level of the antibody molecule that specifically binds FcγRIIb and/or the second antibody molecule and/or the third antibody molecule as defined herein, unless otherwise defined, will usually be from 1 mg/kg bodyweight of the patient to 20 mg/kg, or in some cases even up to 100 mg/kg administered in single or divided doses. In some preferred embodiments, the dose of the antibody molecule that specifically binds FcγRIIb will be 10 mg/kg. Lower doses may be used in some circumstances, for example in combination with prolonged administration. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this disclosure.

Typically, the composition and/or medicament of the disclosure will contain the antibody molecule that specifically binds FcγRIIb at a concentration of between approximately 2 mg/ml and 150 mg/ml or between approximately 2 mg/ml and 200 mg/ml. In a preferred embodiment, the medicaments and/or compositions of the disclosure will contain the antibody molecule that specifically binds FcγRIIb at a concentration of 10 mg/ml.

Generally, in humans, oral or parenteral administration of the composition, and/or antibody, and/or agent, and/or medicament of the disclosure is the preferred route, being the most convenient. For veterinary use, the composition, and/or antibody, and/or agent and/or medicament of the disclosure are administered as a suitably acceptable formulation in accordance with normal veterinary practice and the veterinary surgeon will determine the dosing regimen and route of administration which will be most appropriate for a particular animal. Thus, the present disclosure provides a pharmaceutical formulation comprising an amount of an antibody and/or agent of the disclosure effective to treat various conditions (as described above and further below). Preferably, the composition, and/or antibody, and/or agent, and/or medicament is adapted for delivery by a route selected from the group comprising: intravenous (IV); subcutaneous (SC), intramuscular (IM), or intratumoural. In some preferred embodiments, administration is intravenous.

In some embodiments, either the first antibody molecule or the second antibody or both may be administered through the use of plasmids or viruses. Such plasmids then comprise nucleotide sequences encoding either the first antibody molecule or the second antibody or both. In some embodiments, nucleotide sequences encoding parts of or the full sequences of either the first antibody molecule or the second antibody or both integrated in a cell or viral genome or in a viriome in a virus; such a cell or virus then act as a delivery vehicle for either the first antibody molecule or the second antibody or both (or a delivery vehicle for a nucleotide sequence encoding either the first antibody molecule or the second antibody or both). For example, in some embodiments, such a virus may be in the form of a therapeutic oncolytic virus comprising nucleotide sequences encoding at least one of the antibody molecules described herein. In some embodiments, such an oncolytic virus comprises nucleotide sequences encoding a full-length human IgG antibody. Oncolytic viruses are known to those skilled in the arts of medicine and virology.

The present disclosure also includes composition, and/or antibody, and/or agent, and/or medicament comprising pharmaceutically acceptable acid or base addition salts of the polypeptide binding moieties of the present disclosure The acids which are used to prepare the pharmaceutically acceptable acid addition salts of the aforementioned base compounds useful in this disclosure are those which form non-toxic acid addition salts, i.e. salts containing pharmacologically acceptable anions, such as the hydrochloride, hydrobromide, hydroiodide, nitrate, sulphate, bisulphate, phosphate, acid phosphate, acetate, lactate, citrate, acid citrate, tartrate, bitartrate, succinate, maleate, fumarate, gluconate, saccharate, benzoate, methanesulphonate, ethanesulphonate, benzenesulphonate, p-toluenesulphonate and pamoate [i.e. 1,1′-methylene-bis-(2-hydroxy-3 naphthoate)] salts, among others. Pharmaceutically acceptable base addition salts may also be used to produce pharmaceutically acceptable salt forms of the agents according to the present disclosure. The chemical bases that may be used as reagents to prepare pharmaceutically acceptable base salts of the present agents that are acidic in nature are those that form non-toxic base salts with such compounds. Such non-toxic base salts include, but are not limited to those derived from such pharmacologically acceptable cations such as alkali metal cations (e.g. potassium and sodium) and alkaline earth metal cations (e.g. calcium and magnesium), ammonium or water-soluble amine addition salts such as N-methylglucamine-(meglumine), and the lower alkanolammonium and other base salts of pharmaceutically acceptable organic amines, among others. The agents and/or polypeptide binding moieties of the disclosure may be lyophilised for storage and reconstituted in a suitable carrier prior to use. Any suitable lyophilisation method (e.g. spray drying, cake drying) and/or reconstitution techniques can be employed. It will be appreciated by those skilled in the art that lyophilisation and reconstitution can lead to varying degrees of antibody activity loss (e.g. with conventional immunoglobulins, IgM antibodies tend to have greater activity loss than IgG antibodies) and that use levels may have to be adjusted upward to compensate. In one embodiment, the lyophilised (freeze dried) polypeptide binding moiety loses no more than about 20%, or no more than about 25%, or no more than about 30%, or no more than about 35%, or no more than about 40%, or no more than about 45%, or no more than about 50% of its activity (prior to lyophilisation) when re-hydrated.

The first antibody as defined herein in relation to the eighth to fourteenth aspects may be the same antibody as defined herein in relation to the preceding aspects. All of the embodiments and examples relating to the identity and antibody sequence of the first antibody apply equally to the disclosure as described in the eighth to fourteenth aspects.

The second antibody as defined herein in relation to the eighth to fourteenth aspects may be the same antibody as the third antibody molecule defined herein in relation to the preceding first to seventh aspects. All of the embodiments and examples relating to the identity of the third antibody that specifically binds to CTLA-4 apply equally to the disclosure as described in the eighth to fourteenth aspects insofar as it relates to the second antibody molecule of these aspects.

Antibody molecules as referred to herein (i.e. the first antibody molecule, the second antibody molecule and the third antibody molecule) are well known to those skilled in the art of immunology and molecular biology. Typically, an antibody comprises two heavy (H) chains and two light (L) chains. Herein, we sometimes refer to this complete antibody molecule as a full-size or full-length antibody. The antibody's heavy chain comprises one variable domain (VH) and three constant domains (CH1, CH2 and CH3), and the antibody's molecule light chain comprises one variable domain (VL) and one constant domain (CL). The variable domains (sometimes collectively referred to as the Fv region) bind to the antibody's target, or antigen. Each variable domain comprises three loops, referred to as complementary determining regions (CDRs), which are responsible for target binding. The constant domains are not involved directly in binding an antibody to an antigen, but exhibit various effector functions. Depending on the amino acid sequence of the constant region of their heavy chains, antibodies or immunoglobulins can be assigned to different classes. There are five major classes of immunoglobulins: IgA, IgD, IgE, IgG and IgM, and in humans several of these are further divided into subclasses (isotypes), e.g., IgG1, IgG2, IgG3, and IgG4; IgA1 and IgA2.

Another part of an antibody is the Fc region (otherwise known as the fragment crystallisable domain), which comprises two of the constant domains of each of the antibody's heavy chains. As mentioned above, the Fc region is responsible for interactions between the antibody and Fc receptor.

The term antibody molecule, as used herein, encompasses full-length or full-size antibodies as well as functional fragments of full length antibodies and derivatives of such antibody molecules.

Functional fragments of a full-size antibody have the same antigen binding characteristics as the corresponding full-size antibody and include either the same variable domains (i.e. the VH and VL sequences) and/or the same CDR sequences as the corresponding full-size antibody. That the functional fragment has the same antigen binding characteristics as the corresponding full-size antibody means that it binds to the same epitope on the target as the full-size antibody. Such a functional fragment may correspond to the Fv part of a full-size antibody. Alternatively, such a fragment may be a Fab, also denoted F(ab), which is a monovalent antigen-binding fragment that does not contain a Fc part, or a F(ab′)₂, which is an divalent antigen-binding fragment that contains two antigen-binding Fab parts linked together by disulfide bonds, or a F(ab′), i.e. a monovalent-variant of a F(ab′)₂. Such a fragment may also be single chain variable fragment (scFv).

A functional fragment does not always contain all six CDRs of a corresponding full-size antibody. It is appreciated that molecules containing three or fewer CDR regions (in some cases, even just a single CDR or a part thereof) are capable of retaining the antigen-binding activity of the antibody from which the CDR(s) are derived. For example, in Gao et al., 1994, J. Biol. Chem., 269: 32389-93 it is described that a whole VL chain (including all three CDRs) has a high affinity for its substrate.

Molecules containing two CDR regions are described, for example, by Vaughan & Sollazzo 2001, Combinatorial Chemistry & High Throughput Screening, 4: 417-430. On page 418 (right column—3 Our Strategy for Design) a minibody including only the H1 and H2 CDR hypervariable regions interspersed within framework regions is described. The minibody is described as being capable of binding to a target. Pessi et al., 1993, Nature, 362: 367-9 and Bianchi et al., 1994, J. Mol. Biol., 236: 649-59 are referenced by Vaughan & Sollazzo and describe the H1 and H2 minibody and its properties in more detail. In Qiu et al., 2007, Nature Biotechnology, 25:921-9 it is demonstrated that a molecule consisting of two linked CDRs are capable of binding antigen. Quiocho 1993, Nature, 362: 293-4 provides a summary of “minibody” technology. Ladner 2007, Nature Biotechnology, 25:875-7 comments that molecules containing two CDRs are capable of retaining antigen-binding activity.

Antibody molecules containing a single CDR region are described, for example, in Laune et al., 1997, JBC, 272: 30937-44, in which it is demonstrated that a range of hexapeptides derived from a CDR display antigen-binding activity and it is noted that synthetic peptides of a complete, single, CDR display strong binding activity. In Monnet et al., 1999, JBC, 274: 3789-96 it is shown that a range of 12-mer peptides and associated framework regions have antigen-binding activity and it is commented on that a CDR3-like peptide alone is capable of binding antigen. In Heap et al., 2005, J. Gen. Virol., 86: 1791-1800 it is reported that a “micro-antibody” (a molecule containing a single CDR) is capable of binding antigen and it is shown that a cyclic peptide from an anti-HIV antibody has antigen-binding activity and function. In Nicaise et al., 2004, Protein Science, 13:1882-91 it is shown that a single CDR can confer antigen-binding activity and affinity for its lysozyme antigen.

Thus, antibody molecules having five, four, three or fewer CDRs are capable of retaining the antigen binding properties of the full-length antibodies from which they are derived.

The antibody molecule may also be a derivative of a full-length antibody or a fragment of such an antibody. When a derivative is used it should have the same antigen binding characteristics as the corresponding full-length antibody in the sense that it binds to the same epitope on the target as the full-length antibody.

Thus, by the term “antibody molecule”, as used herein, we include all types of antibody molecules and functional fragments thereof and derivatives thereof, including: monoclonal antibodies, polyclonal antibodies, synthetic antibodies, recombinantly produced antibodies, multi-specific antibodies, bi-specific antibodies, human antibodies, antibodies of human origin, humanized antibodies, chimeric antibodies, single chain antibodies, single-chain Fvs (scFv), Fab fragments, F(ab′)₂ fragments, F(ab′) fragments, disulfide-linked Fvs (sdFv), antibody heavy chains, antibody light chains, homo-dimers of antibody heavy chains, homo-dimers of antibody light chains, heterodimers of antibody heavy chains, heterodimers of antibody light chains, antigen binding functional fragments of such homo- and heterodimers.

Further, the term “antibody molecule”, as used herein, includes all classes of antibody molecules and functional fragments, including: IgG, IgG1, IgG2, IgG3, IgG4, IgA, IgM, IgD, and IgE, unless otherwise specified.

In some embodiments, the antibody is a human IgG1. The skilled person will appreciate that the mouse IgG2a and human IgG1 engage with activatory Fe gamma receptors, and share the ability to activate deletion of target cells through activation of activatory Fc gamma receptor bearing immune cells by e.g. ADCP and ADCC. As such, in embodiments where the mouse IgG2a is the preferred isotype for deletion in the mouse, human IgG1 is a preferred isotype for deletion in human in such embodiments.

As outlined above, different types and forms of antibody molecules are encompassed by the disclosure, and would be known to the person skilled in immunology. It is well known that antibodies used for therapeutic purposes are often modified with additional components which modify the properties of the antibody molecule.

Accordingly, we include that an antibody molecule of the disclosure or an antibody molecule used in accordance with the disclosure (for example, a monoclonal antibody molecule, and/or polyclonal antibody molecule, and/or bi-specific antibody molecule) comprises a detectable moiety and/or a cytotoxic moiety.

By “detectable moiety”, we include one or more from the group comprising of: an enzyme; a radioactive atom; a fluorescent moiety; a chemiluminescent moiety; a bioluminescent moiety.

The detectable moiety allows the antibody molecule to be visualised in vitro, and/or in vivo, and/or ex vivo.

By “cytotoxic moiety”, we include a radioactive moiety, and/or enzyme, wherein the enzyme is a caspase, and/or toxin, wherein the toxin is a bacterial toxin or a venom; wherein the cytotoxic moiety is capable of inducing cell lysis.

We further include that the antibody molecule may be in an isolated form and/or purified form, and/or may be PEGylated. PEGylation is a method by which polyethylene glycol polymers are added to a molecule such as an antibody molecule or derivative to modify its behaviour, for example to extend its half-life by increasing its hydrodynamic size, preventing renal clearance.

As discussed above, the CDRs of an antibody bind to the antibody target. The assignment of amino acids to each CDR described herein is in accordance with the definitions according to Kabat E A et al. 1991, In “Sequences of Proteins of Immunological Interest” Fifth Edition, NIH Publication No. 91-3242, pp xv-xvii.

As the skilled person would be aware, other methods also exist for assigning amino acids to each CDR. For example, the International ImMunoGeneTics information system (IMGT(R)) (http://www.imgt.org/and Lefranc and Lefranc “The Immunoglobulin FactsBook” published by Academic Press, 2001).

In a further embodiment, the antibody molecule of the present disclosure or used according to the disclosure is an antibody molecule that is capable of competing with the specific antibodies provided herein, for example antibody molecules comprising any of the amino acid sequences set out in for example SEQ ID NOs: 1-194 for binding to the specific target.

By “capable of competing for” we mean that the competing antibody is capable of inhibiting or otherwise interfering, at least in part, with the binding of an antibody molecule as defined herein to the specific target.

For example, such a competing antibody molecule may be capable of inhibiting the binding of an antibody molecule described herein by at least about 10%; for example at least about 20%, or at least about 30%, at least about 40%, at least about 50%, at least about 60%, at least about 70%, at least about 80%, at least about 90%, at least about 95%, about 100% and/or inhibiting the ability of the antibody described herein to prevent or reduce binding to the specific target by at least about 10%; for example at least about 20%, at least about 30%, at least about 40%, at least about 50%, at least about 60%, at least about 70%, at least about 80%, at least about 90%, at least about 95%, or about 100%.

Competitive binding may be determined by methods well known to those skilled in the art, such as Enzyme-linked immunosorbent assay (ELISA).

ELISA assays can be used to evaluate epitope-modifying or blocking antibodies. Additional methods suitable for identifying competing antibodies are disclosed in Antibodies: A Laboratory Manual, Harlow & Lane, which is incorporated herein by reference (for example, see pages 567 to 569, 574 to 576, 583 and 590 to 612, 1988, CSHL, NY, ISBN 0-87969-314-2).

It is well known that an antibody specifically binds to or interacts with a defined target molecule or antigen. That is to say, the antibody preferentially and selectively binds its target and not a molecule which is not a target.

Methods of assessing protein binding are known to the person skilled in biochemistry and immunology. It would be appreciated by the skilled person that those methods could be used to assess binding of an antibody to a target and/or binding of the Fc region of an antibody to an Fc receptor; as well as the relative strength, or the specificity, or the inhibition, or prevention, or reduction in those interactions. Examples of methods that may be used to assess protein binding are, for example, immunoassays, BIAcore, western blots, radioimmunoassay (RIA) and enzyme-linked immunosorbent assays (ELISAs) (See Fundamental Immunology Second Edition, Raven Press, New York at pages 332-336 (1989) for a discussion regarding antibody specificity).

Accordingly, by “antibody molecule that specifically binds” we include that the antibody molecule specifically binds a target but does not bind to non-target, or binds to a non-target more weakly (such as with a lower affinity) than the target.

We also include the meaning that the antibody specifically binds to the target at least two-fold more strongly, or at least five-fold more strongly, or at least 10-fold more strongly, or at least 20-fold more strongly, or at least 50-fold more strongly, or at least 100-fold more strongly, or at least 200-fold more strongly, or at least 500-fold more strongly, or at least than about 1000-fold more strongly than to a non-target.

Additionally, we include the meaning that the antibody specifically binds to the target if it binds to the target with a K_{d} of at least about 10⁻¹ K_{d}, or at least about 10⁻² K_{d}, or at least about 10⁻³ K_{d}, or at least about 10⁻⁴ K_{d}, or at least about 10⁻⁵ K_{d}, or at least about 10⁻⁶ K_{d}, or at least about 10⁻⁷ K_{d}, or at least about 10⁻⁸ K_{d}, or at least about 10⁻⁹ K_{d}, or at least about 10⁻¹⁰ K_{d}, or at least about 10⁻¹¹ K_{d}, or at least about 10⁻¹² K_{d}, or at least about 10⁻¹³ K_{d}, or at least about 10⁻¹⁴ K_{d}, or at least about 10⁻¹⁵ K_{d}.

Preferred, non-limiting examples which embody certain aspects of the disclosure will now be described, with reference to the following figures and examples:

### BRIEF DESCRIPTION OF THE DRAWINGS

FIG. 1: Fc:FcγR-binding proficient anti-FcγRIIB (AT-130-2 mIgG2a and mIgG1), but not Fc:FcγR-binding impaired anti-FcγRIIB (AT-130-2 mIgG1 NA), enhances anti-PD-1 antibody therapeutic efficacy and survival in vivo. MC38 tumor-bearing mice were treated three times (days 8, 12 and 15 post inoculation of 5×10⁵ tumor cells S.C. in 1001 PBS) with 200pg of anti-PD-1 (Clone 29F.1A12; Bioxcell) antibody alone or in combination with 200 pg indicated anti-FcγRIIB antibody variant or isotype control (WR17). For the first treatment AT 130-2 was administered 6 hours prior to anti-PD1 antibody. For subsequent treatments both antibodies were given together. All injections were I. P. in 200 μl PBS. Tumors were considered terminal when they reached an area of 225 mm² for MC38. Graphs show tumor growth and survival of animals. (**P<0.01; Log-Rank test). The experiments were done in female mice aged 8-14 weeks.

FIG. 2: Fc:FcγR-binding proficient anti-FcγRIIB (AT-130-2 mIgG2a and mIgG1), but not Fc:FcγR-binding impaired anti-FcγRIIB (AT-130-2 mIgG1 NA), enhances anti-PD-1 antibody therapeutic efficacy and survival in vivo. CT26 tumor-bearing mice were treated three times (days 8, 12 and 15 post inoculation of 5×10⁵ tumor cells S.C. in 100 μl PBS) with 200pg of anti-PD-1 (Clone 29F.1A12; Bioxcell) antibody alone or in combination with 200 pg indicated anti-FcγRIIB antibody variant or isotype control (WR17). For the first treatment AT 130-2 was administered 6 hours prior to anti-PD1 antibody. For subsequent treatments both antibodies were given together. All injections were I. P. in 200 μl PBS. Tumors were considered terminal when they reached an area of 400 mm² for CT26. Graphs show tumor growth and survival of animals. (**P<0.01; Log-Rank test). The experiments were done in female mice aged 8-14 weeks.

FIG. 3: Combined treatment with α-CTLA-4 and the BI-1607 surrogate AT130-2 mIgG1 N297A results in enhanced survival in the MC38 tumor model. 1×10⁶ MC38 cells were injected subcutaneously (s.c.) into C57BL/6 mice and allowed to establish. Mice were treated once tumors reached a diameter of 6 mm. Mice were treated with 200 g anti-FcγRIIB antibody (clone AT130-2) as a mIgG2a, or 400 g mIgG1 N297A and/or 200 g anti-CTLA-4 antibody (clone 9H10). Mice were subsequently treated 3 and 7 days after the first dose with single or combined mAbs. All antibodies were given intraperitoneally (i.p.) in 200j1 PBS. Tumors were measured 3 times a week with sizes presented as the tumor volume (width²×length×0.52) for individual mice in each treatment group. N═X per group. The figure is a summary of X different experiments.

FIG. 4: Combination therapy with anti-CTLA-4 and FcγRIIB blockade. 5×10⁵ CT26 cells were injected S.C into female BALB/c mice. Mice were randomised into treatment groups when tumour width×length was approximately 100 mm². Treatment was performed on days 0, 2, 4 and 11. 9H10 (hamster anti-mouse CTLA4) only mice received 200 pg antibody I. P in 200 μl PBS on each day. On day 0 combination mice received 100 μg AT130-2 N297A (anti-mouse CD32) in 200 μl PBS IP, 6 hours later they received 200 μg 9H10 I. P in 200 μl PBS. On days 2, 4 and 11 combination mice received both antibodies (200 μg 9H10 and 100 μg AT130-2 NA) in a single 200 μl I. P injection. The width and length of tumours was measured and mice were culled when tumour length×width exceeded 400 mm². Fig. A) represents the treatment schedule. Group 1: No Ab; Group 2: anti-mCD32 (AT130-2 NA; 100 μg); Group 3: anti-CTLA-4 (9H10; 200 μg); Group 4: combination (PC61) 6 hours after AT130-2. Tumours were allowed to establish and were treated at 100 mm². An extra dose was given on day 12. Fig. B) shows growth of the individual tumours. Fig. C) represents the mean tumour area+/−SD or SEM. Fig. D) represents animal survival. Fig. E) Composite from 2 separate experiments (n=10/group) displaying survival and demonstrated that the combination of 9H10 and AT130-2NA (NA combo) is significantly more potent at extending survival than the 9H10 alone (p=0.0179).

FIG. 5: Mice were inoculated with MC38 tumor cells and injected with antibodies once the tumors reached a size of approximately 7×7 mm. 24 h after 3 antibody injections, at day 7-8 after treatment start, mice were sacrificed, and tumors harvested. Tumor single cell suspensions were analyzed for immune cell content by FACS. FIG. 5 A-C show the percentage of different cell populations in the tumor and FIG. 5 D the CD8+/Treg ratio. Combined treatment with anti-CTLA4 and BI-1607 surrogate (AT130-2 mIgG1 N297A) results in decreased number of CD4+/CD25+ cells and an improved CD8/Treg ratio.

FIG. 6: After 10-12 days, the spleens were removed from mice, single cell suspension prepared and then injected i.p. into SCID mice (10-15×10⁶/mice). After 1 h the SCID mice were treated i.p. with 10 mg/kg of either Yervoy, anti-CD25 (basiliximab), Yervoy+BI-1607 surrogate (AT130-2 mIgG1 N297A) or isotype control mAb. 24 hours after antibody injection, i.p. fluid was collected from mice and the cells in the fluid were analyzed using FACS. FIG. 6 A shows the percentage of stained Tregs defined as CD45⁺CD3⁺CD4⁺CD25⁺CD127^{low/neg} out of the total numbers of human CD45+ cells. FIG. 6 B shows the percentage of effector T cells (CD8⁺) of the total number of human CD45+ cells. FIG. 6 C shows the CD8+/Treg ratio. When Yervoy is combined with the BI-1607 surrogate (AT130-2 mIgG1 N297A) the percentage of Tregs is decreased, the percentage of CD8+ increased and the CD8+/Treg ratio improved compard to Yervoy alone. n=4-5 per group.

FIG. 7: Assessment of Treg deletion with anti-IL2R mAb+/−FcγRIIB blockade with wild-type or NA mutant mAb. WT AT130-2 does not appear to give any improvement in deletion; whereas NA variant does. 100 μg AT130-2 NA or mIgG1 WT AT130-2 was given i.p. to female Balb/c mice. 100 μg PC61 given i.p 6 hours later. Tregs (FoxP3+) in spleen determined by FACs 4 days later. Mice were culled and single cell suspensions obtained from the spleen which was stained with antibodies against CD4, CD8 and B220 prior to intracellular FoxP3 staining before being analysed on a FACs canto. The white cell count for each tissue was determined. Tregs were defined as being CD8-CD4+FoxP3+ and the number of Tregs calculated using the white cell count. There was a significantly lower number of Tregs in the spleen of mice 4 days receiving the N297A antibody in combination with PC61 compared to a wild-type mIgG1 AT130-2 (unpaired T-test, P=0.044).

FIG. 8: Combined treatment with α-CTLA-4 and the BI-1607 surrogate AT130-2 mIgG1 N297A, results in enhanced efficacy and retained survival with lower dose of anti-CTLA-4. 1×10⁶ MC38 cells were injected s.c. into C57BL/6 mice and allowed to establish. Mice were treated once tumors reached a diameter of 6 mm. Mice were treated with 2 or 0.4 mg/kg of anti-CTLA-4 (clone 9H10) alone or in combination with 20 mg/kg of anti-FcγRIIB antibody (clone AT130-2) as mIgG1 N297A, 10 mg/kg of anti-CTLA-4, 20 mg/kg of AT130-2) as mIgG1 N297A or with isotype control. Mice were subsequently treated 3 and 7 days after the first dose with single or combined mAbs. All antibodies were given i.p. in 200 μl PBS. 8A Tumors were measured 3 times a week with sizes presented as the tumor volume (width 2×length×0.52) for individual mice in each treatment group. N=10 per group. 8B survival of the different groups of mice (not all groups shown).

FIG. 9: Addition of the BI-1607 surrogate AT130-2 mIgG1 N297A to combined CTLA-4/PD-1 treatment, results in enhanced therapeutic efficacy in the treatment resistant B16 model. 1×10⁶ B16 cells were injected s.c. into C57BL/6 mice. Mice were treated 4 days post tumor inoculation. Mice were treated with 10 mg/kg anti-PD-1+2 or 0.4 mg/kg of anti-CTLA-4 (clone 9H10) alone or in combination with 20 mg/kg of anti-FcγRIIB antibody (clone AT130-2) as mIgG1 N297A. In addition, control groups were treated with 10 mg/kg of anti-PD-1, 10 mg/kg of anti-PD-1+20 mg/kg of AT130-2-N297A or with isotype control. Mice were subsequently treated 3 and 7 days after the first dose with single or combined mAbs. All antibodies were given i.p. in 200 μl PBS. Tumors were measured 3 times a week with sizes presented as the tumor volume (width 2×length×0.52) for individual mice in each treatment group. N=4-10 per group.

FIG. 10: Addition of the BI-1607 surrogate AT130-2 mIgG1 N297A to combined CTLA-4/PD-1 treatment, results in enhanced survival in the treatment resistant B16 model. 1×10⁶ B16 cells were injected s.c. into C57BL/6 mice. Mice were treated 4 days post tumor inoculation. Mice were treated with 10 mg/kg anti-PD-1+2 or 0.4 mg/kg of anti-CTLA-4 (clone 9H10) alone or in combination with 20 mg/kg of anti-FcγRIIB antibody (clone AT130-2) as mIgG1 N297A. In addition, control groups were treated with 10 mg/kg of anti-PD-1, 10 mg/kg of anti-PD-1+20 mg/kg of AT130-2-N297A or with isotype control. Mice were subsequently treated 3 and 7 days after the first dose with single or combined mAbs. All antibodies were given i.p. in 200 μl PBS. Tumors were measured 3 times a week until they reached a size that was predetermined to be the ethical endpoint. N=4-10 per group.

### EXAMPLES

### Background

Generation of mFcγRIIB-Blocking Surrogate mAb

The inventors have previously generated human antibodies capable of effectively blocking the inhibitory FcγRIIB. Two antibody variants derived from the hFcγRIIB-specific antibody 6G11 were generated, a hIgG1 with a wild-type Fc domain proficient in binding both activating and inhibitory FcγRs, and a hIgG1_{N297A} with severely impaired Fc-binding to all FcγRs [24].

To assess the therapeutic potential of blocking FcγRs to enhance activity of immune modulatory antibodies e.g. anti-PD-1 and anti-CTLA-4 in solid cancer, the inventors previously generated surrogate mouse FcγRIIB-blocking antibodies suitable for study in immune competent syngeneic mouse tumour models.

Fc:FcγR-proficient and -deficient blocking antibodies, matching the human lead clinical candidate antibodies to FcγRIIB, were constructed by fusing Fv-sequences of the mouse FcγRIIB-specific antibody AT-130 to mouse IgG2a (Fc:FcγR-proficient) and mouse IgG1N297A (Fc:FcγR-deficient) constant domains, respectively. Anti-FcγR mIgG1 isotype antibodies, which bind only to one of the activating FcγRs (mFcγRIII) and the inhibitory mFcγRII and therefore show “intermediary” Fc:FcγR-binding capacity, were additionally generated. All generated antibodies showed highly specific, high affinity, Fv-mediated binding to mouse FcγRIIB as assessed in ELISA with recombinant protein, in vitro cellular binding and blocking assays [25].

Materials and Methods

Cells

The MC38 and CT26 murine colon carcinoma cell lines and the B16 murine melanoma cell line were obtained from ATCC. Cells were maintained in RPMI 1640 medium containing 2 mM L-glutamine supplemented with 10% Fetal Calf Serum (FCS). Logarithmic growth phase of cells was ensured before harvesting cells for grafting.

Human PBMCs (Hospital of Halmstad) were isolated using Ficoll Paque PLUS and after washing the cells were re-suspended in sterile PBS at 75×10⁶ cells/ml.

Test and Control Substances

The anti-murine CTLA-4 clone 9H10 and anti-murine PD-1 clone 29F.1A12 were purchased from Bioxcell, and Yervoy (ipilimumab) and Basiliximab were purchased from Apoteket. The AT130-2 (anti-FcγRIIB) antibodies were purified from hybridomas. The isotype variants of AT130-2 and isotype control antibodies were transiently expressed in HEK293 cells.

The specificity of the purified research batches was demonstrated in a luminescence-based ELISA or FACS analyses. Endotoxin-levels of antibodies were found to be <0.1 IU/mL as determined by the LAL-Amoebocyte test.

Mouse Models

Subcutaneous MC38 Tumour Model

Six to eight week-old (17-20 g) female C57/BL6 mice (n=10) (bred at Taconic, Denmark). One million (1×10⁶) of MC38 tumour cells in 100 μL of PBS were subcutaneously injected into the flank. Treatment was initiated when tumours reached a diameter of 6×6-8×8 mm (measured by a calliper) (day 1). Treatment was initiated (day 1) according to the treatment schedules below.

Subcutaneous CT26 Tumour Model

Six to eight week-old (17-20g) female Balb c mice (n=10) (bred at Southampton University with original breeders from Charles River). 5×10⁵ of CT26 tumour cells in 100 μL of PBS were subcutaneously injected into the flank. Treatment was initiated when tumours reached a diameter of x mm in diameter (measured by a calliper) (day 1).

Subcutaneous B16 Tumour Model

Six to eight week-old (17-20 g) female C57/BL6 mice (n=10) were obtained from Taconic. One million (1×10⁶) of B16 tumour cells in 100 μL of PBS were subcutaneously injected into the flank. Treatment was initiated 4 days after tumour cell inoculation (day 1) according to the treatment schedule below.

NOG-PBMC-SCID Mouse Model

NOG mice were injected intravenously (i.v.) with 15-20×10⁶ PBMC cells. Two weeks after injection, the spleens were isolated and rendered into a single cell suspension. The cells were resuspended in sterile PBS at 50×10⁶ cells/ml. SCID mice were injected intraperitoneally (i.p.) with 200 μl of the suspension corresponding to 1×10⁶ cells/mouse (comprising human T-cells). One hour later, mice were treated with 10 mg/kg of Yervoy, Basiliximab, Bd-1607 surrogate (AT130-2 mIgG1 N297A) or isotype control mAb (according to the second treatment schedule below). The intraperitoneal fluid of the mice was collected after 24 hours. Human T cell subsets were identified and quantified by FACS using following markers: CD45, CD3, CD4, CD8, CD25, CD127 (all from BD Biosciences).

Treatment Schedules

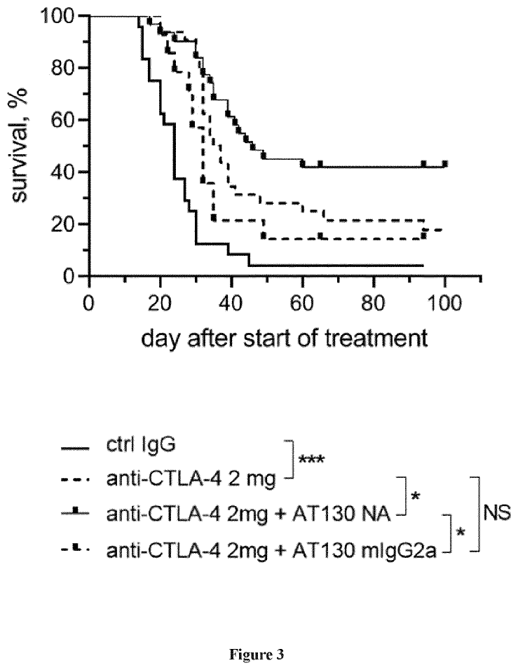

MC38 Model—Anti-CTLA-4 Administration (Example 1, FIG. 3)

| | No. | | Dose | Adm. | |
|---|---|---|---|---|---|
| Groups | mice | Treatment | (mg/kg/inj) | route | Schedule |
| 1 | 10 | anti-FITC (negative control) | 10 | ip | Day 1, 4 and 8 |
| 2 | 10 | Anti-CTLA-4 | 10 | ip | Day 1, 4 and 8 |
| 5 | 10 | Anti-CTLA-4 + AT130-2 | 10 + 10 | ip | Day 1, 4 and 8 |
| | | mIgG2a | | | |
| 6 | 10 | Anti-CTLA-4 + AT130-2 mIgG1 | 10 + 20 | ip | Day 1, 4 and 8 |
| | | N297A | | | |

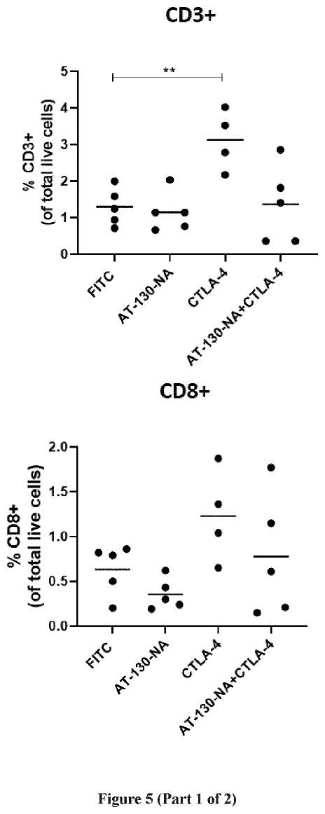

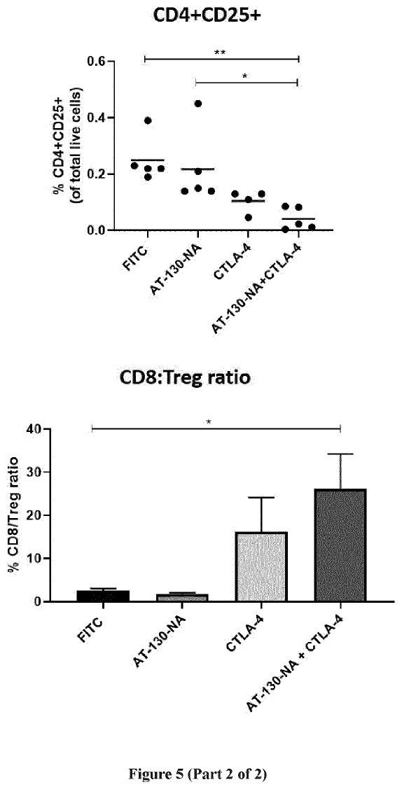

MC38 Model—Tumour Immune Infiltration Study (Example 2, FIG. 5)

| | No. | | Dose | Adm. | |
|---|---|---|---|---|---|
| Groups | mice | Treatment | (mg/kg/inj) | route | Schedule |
| 1 | 5 | FITC isotype control | 10 | ip | Day 1, 4 and 7 |
| 2 | 5 | AT130-2 mIgG1 N297A | 10 | ip | Day 1, 4 and 7 |
| 3 | 5 | Anti-CTLA-4 | 10 | ip | Day 1, 4 and 7 |
| 4 | 5 | IgG2a anti-CTLA-4 + AT130-2 | 10 | ip | Day 1, 4 and 7 |
| | | mIgG1 N297A | | | |

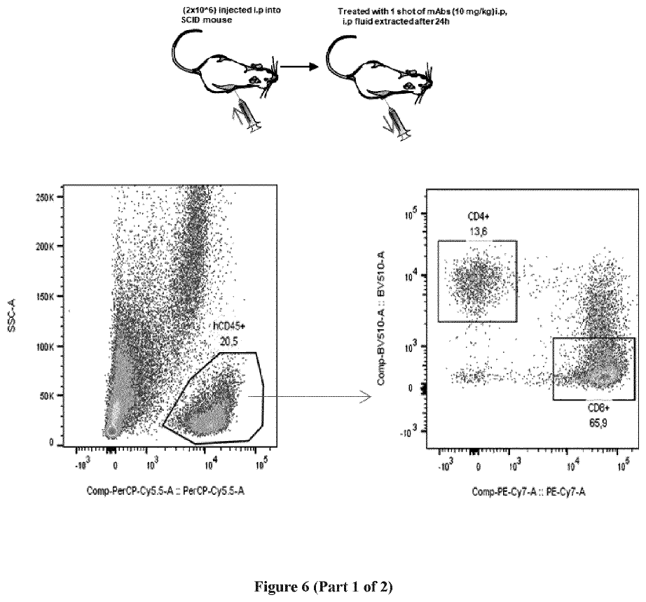

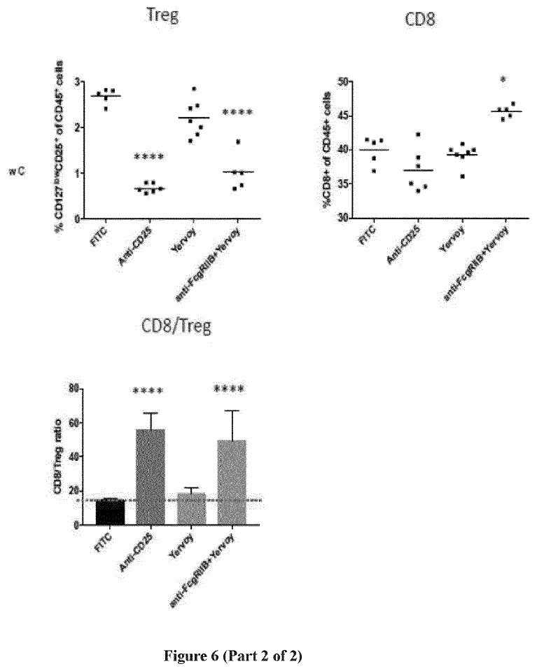

NOG-PBMC-SCID Mouse Model (Example 2, FIG. 6)

| | No. | | Dose | Adm. | |
|---|---|---|---|---|---|
| Groups | mice | Treatment | (mg/kg/inj) | route | Schedule |
| 1 | 7 | Isotype control (FITC) IgG1 | 10 | ip | Day 1 |
| 2 | 7 | Yervoy (ipilimumab) IgG1 | 10 | ip | Day 1 |
| 3 | 7 | Anti-CD25 (Basiliximab) IgG1 | 10 | ip | Day 1 |
| 4 | 7 | Yervoy IgG1 + AT130-2 mIgG1 | 10 | ip | Day 1 |
| | | N297A | | | |

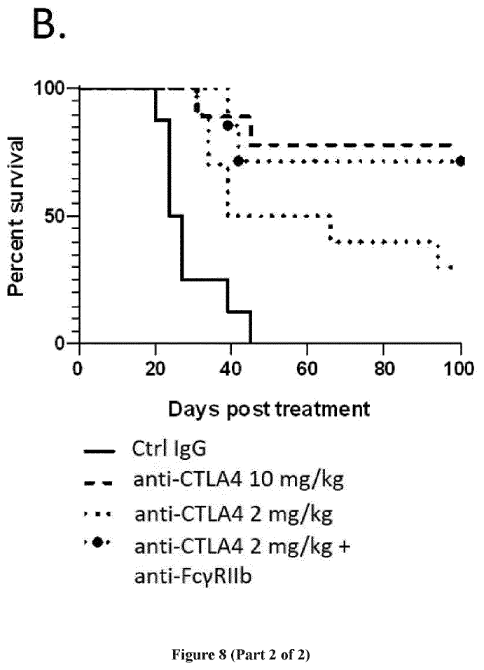

MC38 Model—Anti-CTLA-4 Dose Titration (Example 3, FIG. 8)

| | No. | | Dose | Adm. | |
|---|---|---|---|---|---|
| Groups | mice | Treatment | (mg/kg/inj) | route | Schedule |
| 1 | 10 | Anti-FITC (negative control) | 10 | ip | Day 1, 4 and 8 |
| 2 | 10 | Anti-CTLA-4 | 10 | ip | Day 1, 4 and 8 |
| 3 | 10 | Anti-CTLA-4 | 2 | ip | Day 1, 4 and 8 |
| 4 | 10 | Anti-CTLA-4 | 0.4 | ip | Day 1, 4 and 8 |
| 6 | 10 | Anti-CTLA-4 + AT130-2 mIgG1 | 2 + 10 | ip | Day 1, 4 and 8 |
| | | N297A | | | |
| 7 | 10 | Anti-CTLA-4 + AT130-2 mIgG1 | 0.4 + 10 | ip | Day 1, 4 and 8 |
| | | N297A | | | |

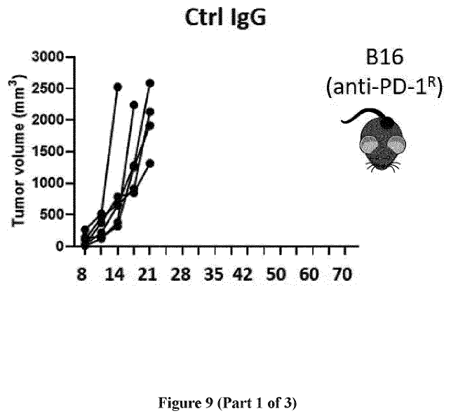

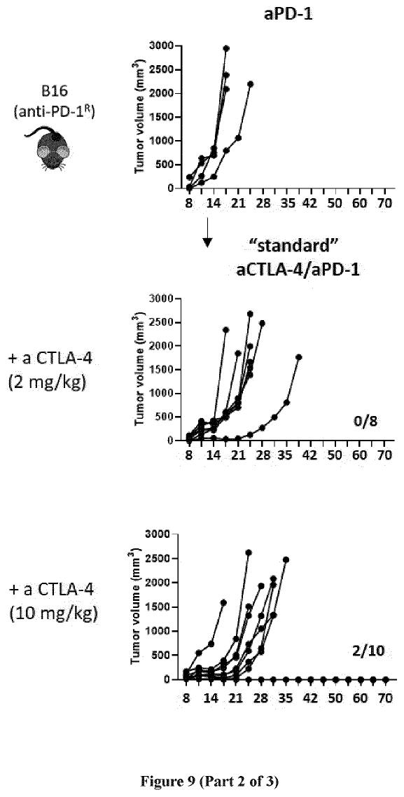

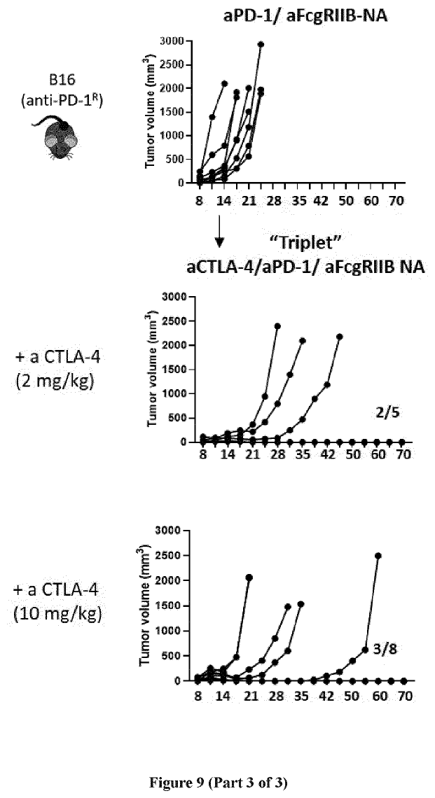

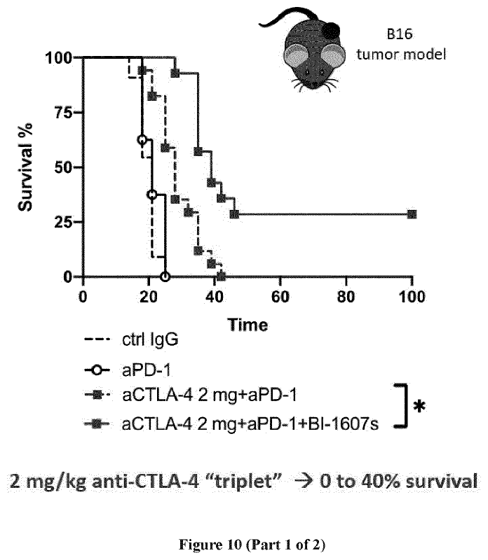

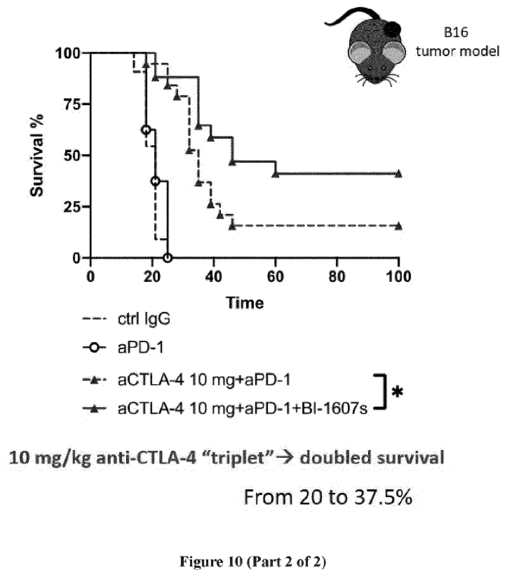

B16 Model—Anti-CTLA-4 and/or Anti-PD-1 in Combination with Anti-FcγRIIb (Example 4, FIGS. 9 and 10)

| | No. | | Dose | Adm. | |
|---|---|---|---|---|---|
| Groups | mice | Treatment | (mg/kg/inj) | route | Schedule |
| 1 | 10 | anti-FITC (negative control) | 10 | ip | Day 1, 4 and 8 |
| 2 | 10 | Anti-PD-1 | 10 | ip | Day 1, 4 and 8 |
| 3 | 10 | Anti-CTLA-4 + anti-PD-1 | 2 + 10 | ip | Day 1, 4 and 8 |
| 4 | 10 | Anti-CTLA-4 + anti-PD-1 | 10 + 10 | ip | Day 1, 4 and 8 |
| 4 | 10 | AT130-2 mIgG1 N297A | 10 | ip | Day 1, 4 and 8 |
| 5 | 10 | Anti-CTLA-4 + anti-PD-1 + | 10 + 10 + | ip | Day 1, 4 and 8 |
| | | AT130-2 mIgG1 N297A | 10 | | |
| 6 | 10 | Anti-CTLA-4 + anti-PD-1 + | 2 + 10 + | ip | Day 1, 4 and 8 |
| | | AT130-2 mIgG1 N297A | 10 | | |

Animal Monitoring

Tumour size was measured twice a week with a calliper and tumour area (width×length) or tumour volume (width²×length×0.52) was calculated.

Animals were euthanized by CO₂ or neck dislocation when tumours reached ethical endpoint or if any of the following occurred:
hunching
scruffy fur
decreased mobility.

Analysis of Tumour Immune-Infiltrates

Tumours were chopped into small pieces and enzymatically digested with a mixture of DNAse and Liberase at 37° C. Further, the tumour solution was filtered through a cell strainer to obtain single cell solution. The cell solution was blocked with IVIG prior to staining. Immune cells were identified and quantified by FACS using following markers: CD45, CD3, CD4, CD8, CD25 (all from BD Biosciences).

Statistical Analysis

Statistical analysis of antibody mediated mouse survival was calculated using log-rank (Mantel-Cox) test (GraphPad Prism). Statistical significance was considered for *=p<0.05, **=p<0.01 ***=p<0.001.

### Example 1—FcγRIIB-Blocking mAb Differentially Modulates Anti-PD-1 and Anti-CTLA-4 Anti-Tumour Activity In Vivo

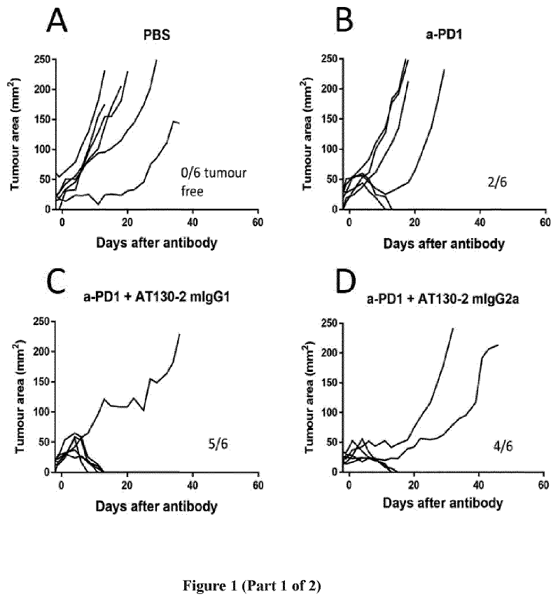

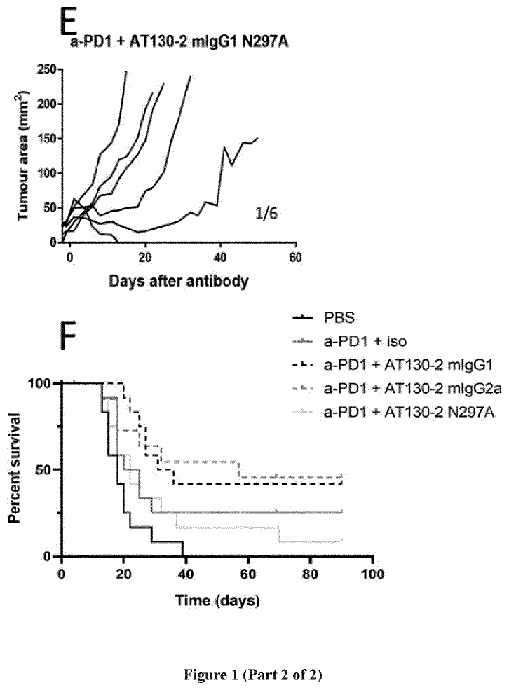

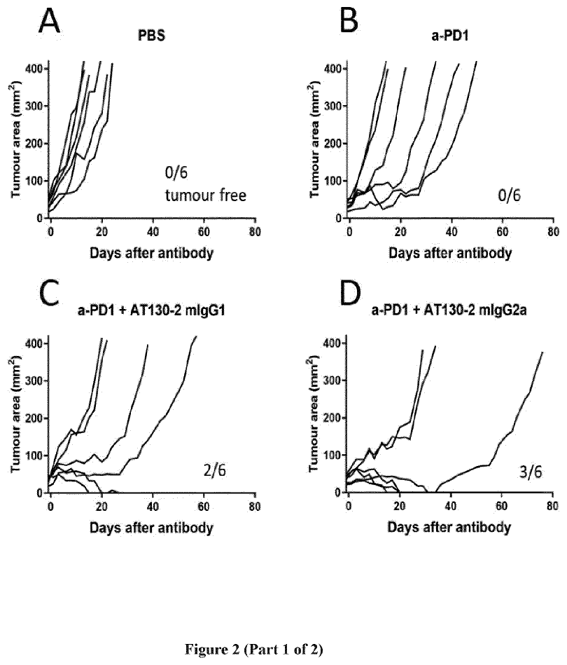

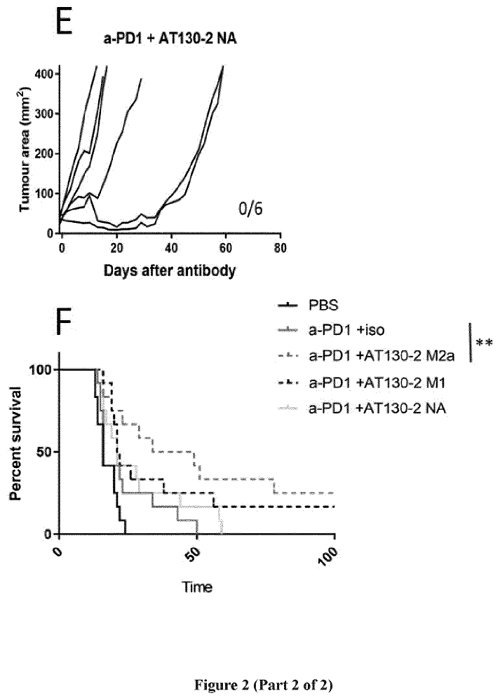

The inventors previously also assessed the ability of Fc:FcγR-proficient and Fc:FcγR-impaired anti-FcγRIIB to enhance anti-PD-1 therapeutic activity in immunocompetent C57BL/6 mice transplanted with syngeneic MC38 or Balb/C mice transplanted with syngeneic CT26 tumours. Both tumour models are known to be infiltrated by immune cells including CD8+ T cells, Treg and macrophages, and to respond partially (MC38) or not (CT26) and anti-PD-1 antibody therapy, reflecting the partial responsiveness observed in human cancer and leaving room to improve efficacy. Strikingly, combination treatment with FcγR-proficient anti-FcγRIIB significantly enhanced anti-tumour activity and survival of anti-PD-1 treated animals in the responsive MC38 model (FIG. 1 A-F) and induced anti-tumour activity and survival in the anti-PD-1 resistant CT26 tumour model (FIG. 2). Conversely, combined treatment with Fc:FcγR-impaired anti-FcγRIIB and anti-PD-1 failed to enhance or even compromised anti-PD-1 therapeutic activity.

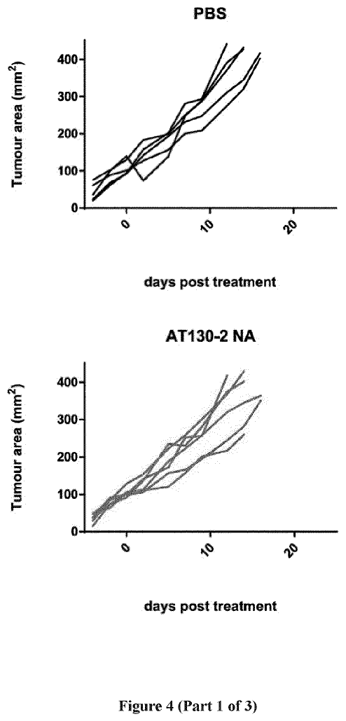

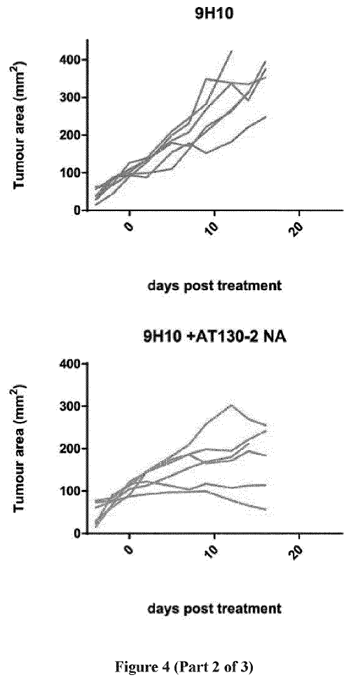

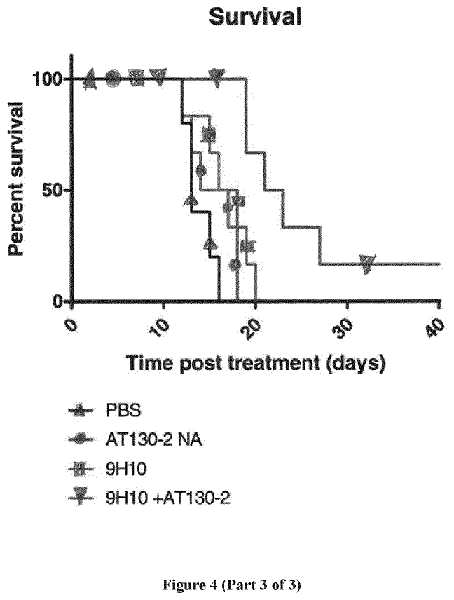

Conversely, and in stark contrast to what was found for anti-PD-1, the inventors have now determined that combination therapy with FcγR-impaired, but not but FcγR-proficient, anti-FcγRIIB enhanced anti-CTLA-4 efficacy as demonstrated by reduced tumour growth and prolonged survival in MC38 tumour-bearing animals (FIG. 3). Similar anti-CTLA-4 enhancing effect of FcR-impaired anti-FcγRIIB were previously observed in CT26 tumour-bearing Balb/c mice (FIG. 4).

Collectively, these findings demonstrated that different variants of anti-FcγRIIB antibodies are needed and useful to enhance in vivo therapeutic activity of immune checkpoint blocking antibodies to CTLA-4 and PD1.

### Example 2—FcγR-Silenced Anti-FcγRIIB Enhancement of Anti-CTLA-4 Anti-Tumour Activity Correlates with Treg Depletion In Vivo

The inventors proceeded to assess cellular mechanisms underlying FcγR-silenced anti-FcγRIIB enhancement of anti-CTLA-4 anti-tumour activity by assessing antibody modulation of tumour infiltrating lymphocytes (TIL). Previous studies had established that anti-CTLA-4 antibody therapy depended on antibody Fc interactions with activating Fc gamma receptors [21] and that improved therapy of antibody constant domains optimized for Fc gamma receptor binding correlated with enhanced Treg depletion and associated higher ratio of intratumoural CD8+: Treg ratios [18].

Further, earlier studies had established that depletion of antibody-coated target cells is coordinately regulated by competition of antibody Fc's for binding to activating and inhibitory (FcγRIIB) FcgRs, which promote and counteract depletion respectively.

Consistent with these observations, treatment with anti-CTLA-4 alone decreased numbers of intratumoural Treg and improved CD8+: Treg ratios (FIG. 5). While treatment with FcγR-silenced anti-FcγRIIB alone did not affect CD8+ T cell or Treg numbers, combined treatment of anti-CTLA-4 with FcγR-silenced anti-FcγRIIB further reduced intratumoural Treg numbers and improved CD8+:Treg ratios (FIG. 5).

Further supporting an in vivo mechanism-of-action involving FcγR-silenced anti-FcγRIIB enhancement of Treg-depletion, combined treatment of PBMC-humanized mice with FcγR-impaired anti-human FcγRIIB (6G11 N297Q) and anti-human CTLA-4 (ipilimumab) resulted in stronger human Treg depletion compared with ipilimumab alone, and resulted in improved human CD8+ T cell:Treg ratios (FIG. 6).

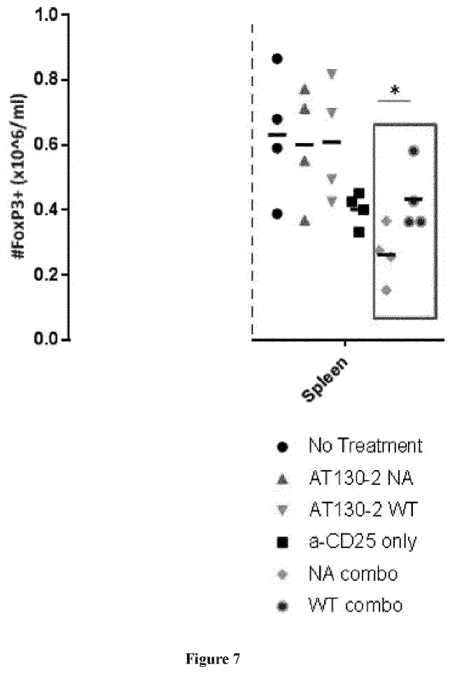

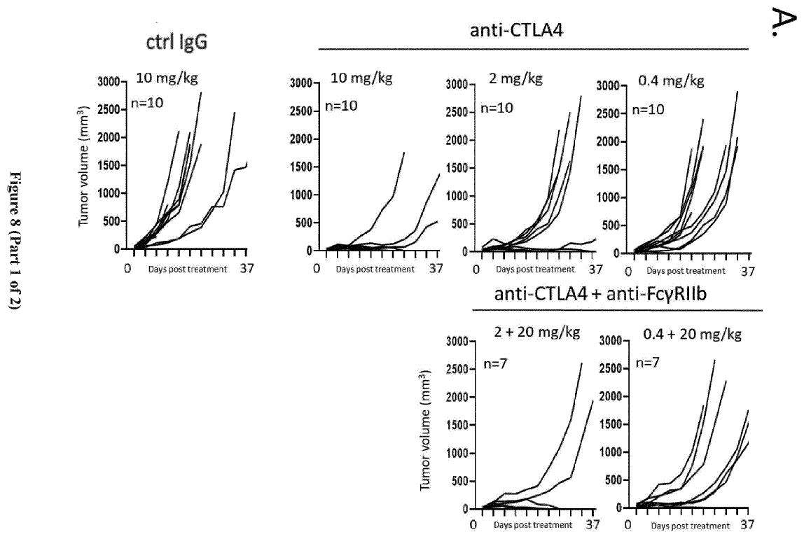

Importantly, this model is characterized by human intratumoural-relevant CTLA-4 expression on both Treg and CD8+T effector cell (FIG. 6). Finally, as has been shown previously, FcγR-silenced anti-FcγRIIB enhanced anti-IL-2 antibody depletion of Treg cells in wild-type C57BL/6 mice (FIG. 7), much as has been observed following genetic deletion of FcγRIIB [26].

Collectively these data suggested that FcγR-silenced anti-FcγRIIB acts to enhance anti-CTLA-4 antibody anti-tumour activity through selective blockade of the inhibitory FcγRIIB, improving activating FcγR-dependent anti-CTLA-4 Treg depletion, and resulting in improved CD8+:Treg ratios. The inventors therefore sought to exploit this finding to determine if this improved the therapeutic window of CTLA-4.

### Example 3—FcγRIIB Blockade Improves Anti-CTLA-4 Therapeutic Window In Vivo

Alongside PD-1 and PD-L1, CTLA-4 remains one of few clinically validated targets for immune checkpoint blockade, and ipilimumab is the only approved anti-CTLA-4 antibody for cancer immunotherapy. Despite anti-CTLA-4 antibodies' ability to induce long-lasting responses, and seemingly cures, in advanced stage cancer patients including melanoma, tolerability concerns, which may be severe and of autoimmune nature, have limited wide-spread use, and resulted in development of therapies comprising lower, sub maximally efficacious, doses. Emerging data indicate that anti-CTLA-4 antibodies may act on both Effector T cells and Treg cells to exert anti-tumour activity. Specifically, blockade of CTLA-4: B7 family interactions and immune inhibitory signaling in CD4+ and CD8+ effector T cells in central compartments is thought to contribute to mounting of anti-CTLA-4-induced adaptive anti-tumour immunity, but may additionally contribute to induction of non-tumour, self-immune responses and autoimmune manifestations [27, 28]. In tumours, anti-CTLA-4 antibodies have been shown to confer Fc gamma receptor-dependent depletion of highly immune suppressive Treg cells, which overexpress CTLA-4 compared to (intratumoural) effector T cells and peripheral Treg cells [18].

As such, enhancing Fc gamma receptor-dependent Treg-depletion of lower better tolerated anti-CTLA-4 doses, may be an attractive strategy to achieve powerful yet safe anti-CTLA-4 antibody immunotherapy.

It is well established that ipilimumab therapeutic activity and toxicity are linked and dose dependent [29]. Accordingly, depending on cancer type and single agent or combination use with anti-PD-1 approved ipilimumab doses span from 1 to 10 mg/kg.

To probe possible immune enhancing effects of FcγR-impaired anti-human FcγRIIB on effective ipilimumab doses that can be safely administered, the inventors treated MC38 tumour-bearing mice with anti-CTLA-4 antibody doses of 2 or 0.4 mg/kg alone or combined with a full therapeutic dose of 10 mg/kg FcγR-impaired anti-human FcγRIIB, and anti-tumour effects were recorded as impaired tumour growth and survival. Treatment with control IgG or a maximally efficacious dose of 10 mg/kg anti-CTLA-4 served as negative and positive controls. Strikingly, when combined with FcγR-impaired anti-human FcγRIIB (BI-1607 surrogate), a five-fold lower dose of ipilimumab (mg) was equally efficacious to the maximally efficacious ipilimumab dose of 10 mg/kg both as assessed by tumour growth inhibition and conferred survival (FIGS. 8A and 8B). Monotherapy with FcγR-impaired anti-human FcγRIIB had no effect on tumour growth or survival compared to control antibody treatment.

These findings demonstrated that FcγR-impaired anti-human FcγRIIB can indeed improve anti-CTLA-4 therapeutic window in vivo.

### Example 4—FcγRIIB Blockade Overcomes Resistance to Immune Checkpoint Blockade with Both Anti-CTLA-4 and Anti-PD-1 In Vivo

While the contribution of ICBs to the patient survival on the whole can hardly be overstated, many patients fail to respond or acquire resistance during the course of therapy. Much remains to be learned about what dictates responsiveness or resistance to ICB, but it is well accepted that patients with immune inflamed tumours are more likely to respond than those with poorly immune infiltrated tumours. Patients with immune excluded or “cold” tumours are unlikely to respond to ICB. Regardless of mechanism, patients resistant to both anti-CTLA-4 and anti-PD-1/PD-L1 have a particularly grave prognosis.

In light of these observations, resistance to ICB constitutes a significant unmet medical need and drugs that could help overcome resistance hold great therapeutic promise. While above studies clearly demonstrated that different types of anti-FcγRIIB antibodies are needed to enhance on anti-CTLA-4 and anti-PD-1 per se, we evaluated potential anti-tumour immunity-enhancing effects of FcγR-impaired anti-FcγRIIB on the combination of anti-CTLA-4 and anti-PD-1. To this end, C57BL/6 mice were transplanted with syngeneic B16 tumour cells, a “cold tumour” type model known to be poorly immune infiltrated and resistant to both anti-CTLA-4 and anti-PD-1 ICB. Consistent with the highly resistant nature of this model, neither treatment with full therapeutic doses of anti-PD-1 (10 mg/kg) alone nor combined treatment with clinically relevant doses of 2 mg/kg anti-CTLA-4 and 10 mg/kg anti-PD-1 afforded survival advantage in this setting (FIGS. 9 and 10). Only the combination of a maximally efficacious CTLA-4 dose of 10 mg/kg combined with 10 mg/kg anti-PD-1 showed anti-tumour activity in this model of highly resistant tumour microenvironments.

Strikingly then, combined treatment with FcγR-impaired anti-FcγRIIB converted the ineffective dose of 2 mg/kg anti-CTLA-4 and 10 mg/kg anti-PD-1 to a highly effective dose that overcame resistance and induced cures in 30% of animals (FIGS. 9 and 10A), and approximately doubled survival of the high (10+10 mg/kg) anti-CTLA-4/anti-PD-1 dosing regimen (FIGS. 9 and 10B).

While doses of mouse surrogate antibodies in mouse tumour models cannot be directly extrapolated to approved human antibodies in cancer subjects, our sum data in the B16/C57BL6 mouse tumour model demonstrate the following. Firstly, consistent with independent reports by independent investigators (such as Jiao et al., Int. J. Mol. Sci., (2020) 21, 773: doi:10.3390/ijms21030773) the B16 model is resistant to full therapeutic, maximally efficacious, doses of anti-CTLA-4 or anti-PD-1, and is resistant to human therapeutically relevant combined doses of (sub-maximally efficacious) anti-CTLA-4 (e.g. 2 mg/kg) and (full therapeutic doses of) anti-PD-1. Second, combined treatment with low dose (2 mg/kg) anti-CTLA-4 and FcγR-impaired anti-FcγRIIB was equally efficacious compared to a maximally efficacious dose (10 mg/kg) of anti-CTLA-4 alone. This demonstrates that FcγR-impaired anti-FcγRIIB improves the therapeutic window of anti-CTLA-4 and indicates that FcγR-impaired anti-FcγRIIB is able to convert a well-tolerated dose of anti-CTLA-4 from submaximal to full therapeutic activity equivalence compared to a (toxic) anti-CTLA-4 single agent treatment. Thirdly, and most importantly, these data demonstrate that combined treatment with FcγR-impaired anti-FcγRIIB and anti-CTLA-4/anti-PD-1 overcomes resistance of “cold tumours” to immune checkpoint blockade.

### REFERENCES

1. Cheson, B. D. and J. P. Leonard, Monoclonal antibody therapy for B-cell non-Hodgkin's lymphoma. N Engl J Med, 2008. 359(6): p. 613-26.
2. Gradishar, W. J., HER2 therapy—an abundance of riches. N Engl J Med, 2012. 366(2): p. 176-8.
3. Jonker, D. J., et al., Cetuximab for the treatment of colorectal cancer. N Engl J Med, 2007. 357(20): p. 2040-8.
4. Lokhorst, H. M., et al., Targeting CD38 with Daratumumab Monotherapy in Multiple Myeloma. N Engl J Med, 2015. 373(13): p. 1207-19.
5. Hodi, F. S., et al., Improved survival with ipilimumab in patients with metastatic melanoma. N Engl J Med, 2010. 363(8): p. 711-23.
6. Larkin, J., F. S. Hodi, and J. D. Wolchok, Combined Nivolumab and Ipilimumab or Monotherapy in Untreated Melanoma. N Engl J Med, 2015. 373(13): p. 1270-1.
7. Brahmer, J. R., et al., Safety and activity of anti-PD-L1 antibody in patients with advanced cancer. N Engl J Med, 2012. 366(26): p. 2455-65.
8. Topalian, S. L., et al., Safety, activity, and immune correlates of anti-PD-1 antibody in cancer. N Engl J Med, 2012. 366(26): p. 2443-54.
9. Ribas, A., et al., Pembrolizumab versus investigator-choice chemotherapy for ipilimumab-refractory melanoma (KEYNOTE-002): a randomised, controlled, phase 2 trial. Lancet Oncol, 2015. 16(8): p. 908-18.
10. Robert, C., et al., Ipilimumab plus dacarbazine for previously untreated metastatic melanoma. N Engl J Med, 2011. 364(26): p. 2517-26.
11. Robert, C., et al., Anti-programmed-death-receptor-1 treatment with pembrolizumab in ipilimumab-refractory advanced melanoma: a randomised dose-comparison cohort of a phase 1 trial. Lancet, 2014. 384(9948): p. 1109-17.
12. Robert, C., et al., Nivolumab in previously untreated melanoma without BRAF mutation. N Engl J Med, 2015. 372(4): p. 320-30.
13. Weber, J. S., et al., Nivolumab versus chemotherapy in patients with advanced melanoma who progressed after anti-CTLA-4 treatment (CheckMate 037): a randomised, controlled, open-label, phase 3 trial. Lancet Oncol, 2015. 16(4): p. 375-84.
14. Sharma, P., et al., Primary, Adaptive, and Acquired Resistance to Cancer Immunotherapy. Cell, 2017. 168(4): p. 707-723.
15. Goede, V., et al., Obinutuzumab plus chlorambucil inpatients with CLL and coexisting conditions. N Engl J Med, 2014. 370(12): p. 1101-10.
16. Baselga, J., et al., Pertuzumab plus trastuzumab plus docetaxel for metastatic breast cancer. N Engl J Med, 2012. 366(2): p. 109-19.
17. Gopal, A. K., et al., PI3Kdelta inhibition by idelalisib inpatients with relapsed indolent lymphoma. N Engl J Med, 2014. 370(11): p. 1008-18.
18. Arce Vargas, F., et al., Fc Effector Function Contributes to the Activity of Human Anti—CTLA-4 Antibodies. Cancer Cell, 2018. 33(4): p. 649-663 e4.
19. Dahan, R., et al., FcgammaRs Modulate the Anti-tumor Activity ofAntibodies Targeting the PD-1 PD-L1 Axis. Cancer Cell, 2015. 28(3): p. 285-95.
20. Arlauckas, S. P., et al., In vivo imaging reveals a tumor-associated macrophage—mediated resistance pathway in anti-PD-1 therapy. Sci Transl Med, 2017. 9(389).
21. Simpson, T. R., et al., Fc-dependent depletion of tumor-infiltrating regulatory T cells co-defines the efficacy of anti-CTLA-4 therapy against melanoma. J Exp Med, 2013. 210(9): p. 1695-710.
22. Sow, H. S., et al., FcgammaR interaction is not required for effective anti-PD-L1 immunotherapy but can add additional benefit depending on the tumor model. Int J Cancer, 2019. 144(2): p. 345-354.
23. Litchfield, K., et al., Meta-analysis of tumor—and T cell-intrinsic mechanisms of sensitization to checkpoint inhibition. Cell, 2021. 184(3): p. 596-614 e14.
24. Roghanian, A., et al., Antagonistic human FcgammaRIIB (CD32B) antibodies have anti-tumor activity and overcome resistance to antibody therapy in vivo. Cancer Cell, 2015. 27(4): p. 473-88.
25. Tutt, A. L., et al., Development and Characterization ofMonoclonal Antibodies Specific for Mouse and Human Fcgamma Receptors. J Immunol, 2015. 195(11): p. 5503-16.
26. Arce Vargas, F., et al., Fc-Optimized Anti-CD25 Depletes Tumor-Infiltrating Regulatory T Cells and Synergizes with PD-1 Blockade to Eradicate Established Tumors. Immunity, 2017. 46(4): p. 577-586.
27. Tivol, E. A., et al., Loss of CTLA-4 leads to massive lymphoprohferation and fatal multiorgan tissue destruction, revealing a critical negative regulatory role of CTLA-4. Immunity, 1995. 3(5): p. 541-7.
28. Waterhouse, P., et al., Lymphoprohferative disorders with early lethality in mice deficient in Ctla-4. Science, 1995. 270(5238): p. 985-8.
29. Bertrand, A., et al., Immune related adverse events associated with anti-CTLA-4 antibodies: systematic review and meta-analysis. BMC Med, 2015. 13: p. 211.

Embodiments of the disclosure are also described in the following numbered paragraphs:
1. A combination comprising:
   a first antibody molecule that specifically binds to FcγRIIb via its Fab region, and that lacks an Fc region or has reduced binding to Fcγ receptors via its Fc region;
   a second antibody molecule that specifically binds to PD-1 or PD-L1; and
   a third antibody molecule that specifically binds to CTLA-4 and that binds to at least one Fcγ receptor via its Fc region;
for use in treating cancer in a patient, wherein the cancer is resistant to treatment with an antibody molecule that specifically binds to PD-1 or PD-L1, and/or an antibody molecule that specifically binds to CTLA-4.
2. Use of:
   a first antibody molecule that specifically binds to FcγRIIb via its Fab region, and that lacks an Fc region or has reduced binding to Fcγ receptors via its Fc region;
   a second antibody molecule that specifically binds to PD-1 or PD-L1; and
   a third antibody molecule that specifically binds to CTLA-4 and that binds to at least one Fcγ receptor via its Fc region;
in the manufacture of a medicament for treating cancer in a patient, wherein the cancer is resistant to treatment with an antibody molecule that specifically binds to PD-1 or PD-L1, and/or an antibody molecule that specifically binds to CTLA-4.
3. A method for treating cancer in a patient, the method comprising administering to the patient:
   a first antibody molecule that specifically binds to FcγRIIb via its Fab region, and that lacks an Fc region or has reduced binding to Fcγ receptors via its Fc region;
   a second antibody molecule that specifically binds to PD-1 or PD-L1; and
   a third antibody molecule that specifically binds to CTLA-4 and that binds to at least one Fcγ receptor via its Fc region;
wherein the cancer is resistant to treatment with an antibody molecule that specifically binds to PD-1 or PD-L1, and/or an antibody molecule that specifically binds to CTLA-4.
4. A first antibody molecule that specifically binds to FcγRIIb via its Fab region, and that lacks an Fc region or has reduced binding to Fcγ receptors via its Fc region, for use in combination with:
   a second antibody molecule that specifically binds to PD-1 or PD-L1; and
   a third antibody molecule that specifically binds to CTLA-4 and that binds to at least one Fcγ receptor via its Fc region;
for treating cancer in a patient, wherein the cancer is resistant to treatment with an antibody molecule that specifically binds to PD-1 or PD-L1, and/or an antibody molecule that specifically binds to CTLA-4.
5. A first antibody molecule that specifically binds to FcγRIIb via its Fab region, and that lacks an Fc region or has reduced binding to Fcγ receptors via its Fc region, for use in treating cancer in a patient;
   characterised in that the first antibody molecule reduces and/or prevents resistance in the cancer to treatment with an antibody molecule that specifically binds to PD-1 or PD-L1, and/or an antibody molecule that specifically binds to CTLA-4.
6. A pharmaceutical composition comprising:
   a first antibody molecule that specifically binds to FcγRIIb via its Fab region, and that lacks an Fc region or has reduced binding to Fcγ receptors via its Fc region;
   a second antibody molecule that specifically binds to PD-1 or PD-L1; and
   a third antibody molecule that specifically binds to CTLA-4. and that binds to at least one Fcγ receptor via its Fc region.
7. A kit comprising:
   a first antibody molecule that specifically binds to FcγRIIb via its Fab region, and that lacks an Fc region or has reduced binding to Fcγ receptors via its Fc region;
   a second antibody molecule that specifically binds to PD-1 or PD-L1; and
   a third antibody molecule that specifically binds to CTLA-4 and that binds to at least one Fcγ receptor via its Fc region.
8. A combination for use, a use, a method, a first antibody molecule for use, a pharmaceutical composition, or a kit according to any one of paragraphs 1-7, wherein the first antibody molecule lacks an Fc region.
9. A combination for use, a use, a method, a first antibody molecule for use, a pharmaceutical composition, or a kit according to any one of paragraphs 1-7, wherein the first antibody molecule has reduced binding to Fcγ receptors via its Fc region, and has an aglycosylated Fc region.
10. A combination for use, a use, a method, a first antibody molecule for use, a pharmaceutical composition, or a kit according to any one of paragraphs 1-9, wherein the first antibody molecule is selected from the group consisting of a human antibody molecule, a humanized antibody molecule, and an antibody molecule of human origin.
11. A combination for use, a use, a method, a first antibody molecule for use, a pharmaceutical composition, or a kit according to any one of paragraphs 1-10, wherein the first antibody molecule is a monoclonal antibody molecule or an antibody molecule of monoclonal origin.
12. A combination for use, a use, a method, a first antibody molecule for use, a pharmaceutical composition, or a kit according to any one of paragraphs 1-11, wherein the first antibody molecule is selected from the group consisting of: a full-length antibody, a chimeric antibody, a single chain antibody, a Fab fragment, a (Fab′)2 fragment, a Fab′ fragment, a (Fab′)2 fragment, a Fv fragment, and an scFv fragment.
13. A combination for use, a use, a method, a first antibody molecule for use, a pharmaceutical composition, or a kit according to any one of paragraphs 1-12, wherein the first antibody molecule is a human IgG antibody molecule having an aglycosylated Fc region or an IgG antibody molecule of human origin having an aglycosylated Fc region.
14. A combination for use, a use, a method, a first antibody molecule for use, a pharmaceutical composition, or a kit according to paragraph 13, wherein the IgG antibody molecule is an IgG1 or IgG2 antibody molecule.
15. A combination for use, a use, a method, a first antibody molecule for use, a pharmaceutical composition, or a kit according to paragraph 14, wherein the IgG antibody molecule is an aglycosylated human IgG1 or an aglycosylated humanized murine antibody or an aglycosylated humanized llama hcIgG antibody or a an aglycosylated chimerised murine IgG.
16. A combination for use, a use, a method, a first antibody molecule for use, a pharmaceutical composition, or a kit according to paragraph 15, wherein the first antibody has been aglycosylated through amino acid substitution in position 297.
17. A combination for use, a use, a method, a first antibody molecule for use, a pharmaceutical composition, or a kit according to paragraph 16, wherein the first antibody has been aglycosylated through an N297Q substitution.
18. A combination for use, a use, a method, a first antibody for use, a pharmaceutical composition, or a kit according to any one of paragraphs 1-17, wherein the first antibody molecule comprises a variable heavy chain (VH) comprising the following CDRs:
   (i) SEQ ID NO: 51 and SEQ ID NO: 52 and SEQ ID NO: 53; or
   (ii) SEQ ID NO: 57 and SEQ ID NO: 58 and SEQ ID NO: 59; or
   (iii) SEQ ID NO: 63 and SEQ ID NO: 64 and SEQ ID NO: 65; or
   (iv) SEQ ID NO: 69 and SEQ ID NO: 70 and SEQ ID NO: 71; or
   (v) SEQ ID NO: 75 and SEQ ID NO: 76 and SEQ ID NO: 77; or
   (vi) SEQ ID NO: 81 and SEQ ID NO: 82 and SEQ ID NO: 83; or
   (vii) SEQ ID NO: 87 and SEQ ID NO: 88 and SEQ ID NO: 89; or
   (viii) SEQ ID NO: 93 and SEQ ID NO: 94 and SEQ ID NO: 95; or
   (ix) SEQ ID NO: 99 and SEQ ID NO: 100 and SEQ ID NO: 101; or
   (x) SEQ ID NO: 105 and SEQ ID NO: 106 and SEQ ID NO: 107; or
   (xi) SEQ ID NO: 111 and SEQ ID NO: 112 and SEQ ID NO: 113; or
   (xii) SEQ ID NO: 117 and SEQ ID NO: 118 and SEQ ID NO: 119; or
   (xiii) SEQ ID NO: 123 and SEQ ID NO: 124 and SEQ ID NO: 125; or
   (xiv) SEQ ID NO: 129 and SEQ ID NO: 130 and SEQ ID NO: 131; or
   (xv) SEQ ID NO: 135 and SEQ ID NO: 136 and SEQ ID NO: 137; or
   (xvi) SEQ ID NO: 141 and SEQ ID NO: 142 and SEQ ID NO: 143; or
   (xvii) SEQ ID NO: 147 and SEQ ID NO: 148 and SEQ ID NO: 149; or
   (xviii) SEQ ID NO: 153 and SEQ ID NO: 154 and SEQ ID NO: 155; or
   (xix) SEQ ID NO: 159 and SEQ ID NO: 160 and SEQ ID NO: 161; or
   (xx) SEQ ID NO: 165 and SEQ ID NO: 166 and SEQ ID NO: 167; or
   (xxi) SEQ ID NO: 171 and SEQ ID NO: 172 and SEQ ID NO: 173; or
   (xxii) SEQ ID NO: 177 and SEQ ID NO: 178 and SEQ ID NO: 179; or
   (xxiii) SEQ ID NO: 183 and SEQ ID NO: 184 and SEQ ID NO: 185; or
   (xxiv) SEQ ID NO: 189 and SEQ ID NO: 190 and SEQ ID NO: 191.
19. A combination for use, a use, a method, a first antibody molecule for use, a pharmaceutical composition, or a kit according to any one of paragraphs 1-18, wherein the first antibody molecule comprises a variable light chain (VL) comprising the following CDRs:
   (i) SEQ ID NO: 54 and SEQ ID NO: 55 and SEQ ID NO: 56; or
   (ii) SEQ ID NO: 60 and SEQ ID NO: 61 and SEQ ID NO: 62; or
   (iii) SEQ ID NO: 66 and SEQ ID NO: 67 and SEQ ID NO: 68; or
   (iv) SEQ ID NO: 72 and SEQ ID NO: 73 and SEQ ID NO: 74; or
   (v) SEQ ID NO: 78 and SEQ ID NO: 79 and SEQ ID NO: 80; or
   (vi) SEQ ID NO: 84 and SEQ ID NO: 85 and SEQ ID NO: 86; or
   (vii) SEQ ID NO: 90 and SEQ ID NO: 91 and SEQ ID NO: 92; or
   (viii) SEQ ID NO: 96 and SEQ ID NO: 97 and SEQ ID NO: 98; or
   (ix) SEQ ID NO: 102 and SEQ ID NO: 103 and SEQ ID NO: 104; or
   (x) SEQ ID NO: 108 and SEQ ID NO: 109 and SEQ ID NO: 110; or
   (xi) SEQ ID NO: 114 and SEQ ID NO: 115 and SEQ ID NO: 116; or
   (xii) SEQ ID NO: 120 and SEQ ID NO: 121 and SEQ ID NO: 122; or
   (xiii) SEQ ID NO: 126 and SEQ ID NO: 127 and SEQ ID NO: 128; or
   (xiv) SEQ ID NO: 132 and SEQ ID NO: 133 and SEQ ID NO: 134; or
   (xv) SEQ ID NO: 138 and SEQ ID NO: 139 and SEQ ID NO: 140; or
   (xvi) SEQ ID NO: 144 and SEQ ID NO: 145 and SEQ ID NO: 146; or
   (xvii) SEQ ID NO: 150 and SEQ ID NO: 151 and SEQ ID NO: 152; or
   (xviii) SEQ ID NO: 156 and SEQ ID NO: 157 and SEQ ID NO: 158; or
   (xix) SEQ ID NO: 162 and SEQ ID NO: 163 and SEQ ID NO: 164; or
   (xx) SEQ ID NO: 168 and SEQ ID NO: 169 and SEQ ID NO: 170; or
   (xxi) SEQ ID NO: 174 and SEQ ID NO: 175 and SEQ ID NO: 176; or
   (xxii) SEQ ID NO: 180 and SEQ ID NO: 181 and SEQ ID NO: 182; or
   (xxiii) SEQ ID NO: 186 and SEQ ID NO: 187 and SEQ ID NO: 188; or
   (xxiv) SEQ ID NO: 192 and SEQ ID NO: 193 and SEQ ID NO: 194.
20. A combination for use, a use, a method, a first antibody molecule for use, a pharmaceutical composition, or a kit according to any one of paragraphs 1-19, wherein the first antibody molecule comprises a variable heavy chain (VH) amino acid sequence selected from the group consisting of: SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; and SEQ ID NO: 26.
21. A combination for use, a use, a method, a first antibody molecule for use, a pharmaceutical composition, or a kit according to any one of paragraphs 1-20, wherein the first antibody molecule comprises a variable light chain (VL) amino acid sequence selected from the group consisting of: SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 38; SEQ ID NO: 39; SEQ ID NO: 40; SEQ ID NO: 41; SEQ ID NO: 42; SEQ ID NO: 43; SEQ ID NO: 44; SEQ ID NO: 45; SEQ ID NO: 46; SEQ ID NO: 47; SEQ ID NO: 48; SEQ ID NO: 49; and SEQ ID NO: 50.
22. A combination for use, a use, a method, a first antibody molecule for use, a pharmaceutical composition, or a kit according to any one of paragraphs 1-21, wherein the first antibody molecule comprises the following CDR amino acid sequences:
   (i) SEQ ID NO: 51 and SEQ ID NO: 52 and SEQ ID NO: 53 and SEQ ID NO: 54 and SEQ ID NO: 55 and SEQ ID NO: 56; or
   (ii) SEQ ID NO: 57 and SEQ ID NO: 58 and SEQ ID NO: 59 and SEQ ID NO: 60 and SEQ ID NO: 61 and SEQ ID NO: 62; or
   (iii) SEQ ID NO: 63 and SEQ ID NO: 64 and SEQ ID NO: 65 and SEQ ID NO: 66 and SEQ ID NO: 67 and SEQ ID NO: 68; or
   (iv) SEQ ID NO: 69 and SEQ ID NO: 70 and SEQ ID NO: 71 and SEQ ID NO: 72 and SEQ ID NO: 73 and SEQ ID NO: 74; or
   (v) SEQ ID NO: 75 and SEQ ID NO: 76 and SEQ ID NO: 77 and SEQ ID NO: 78 and SEQ ID NO: 79 and SEQ ID NO: 80; or
   (vi) SEQ ID NO: 81 and SEQ ID NO: 82 and SEQ ID NO: 83 and SEQ ID NO: 84 and SEQ ID NO: 85 and SEQ ID NO: 86; or
   (vii) SEQ ID NO: 87 and SEQ ID NO: 88 and SEQ ID NO: 89 and SEQ ID NO: 90 and SEQ ID NO: 91 and SEQ ID NO: 92; or
   (viii) SEQ ID NO: 93 and SEQ ID NO: 94 and SEQ ID NO: 95 and SEQ ID NO: 96 and SEQ ID NO: 97 and SEQ ID NO: 98; or
   (ix) SEQ ID NO: 99 and SEQ ID NO: 100 and SEQ ID NO: 101 and SEQ ID NO: 102 and SEQ ID NO: 103 and SEQ ID NO: 104; or
   (x) SEQ ID NO: 105 and SEQ ID NO: 106 and SEQ ID NO: 107 and SEQ ID NO: 108 and SEQ ID NO: 109 and SEQ ID NO: 110; or
   (xi) SEQ ID NO: 111 and SEQ ID NO: 112 and SEQ ID NO: 113 and SEQ ID NO: 114 and SEQ ID NO: 115 and SEQ ID NO: 116; or
   (xii) SEQ ID NO: 117 and SEQ ID NO: 118 and SEQ ID NO: 119 and SEQ ID NO: 120 and SEQ ID NO: 121 and SEQ ID NO: 122; or
   (xiii) SEQ ID NO: 123 and SEQ ID NO: 124 and SEQ ID NO: 125 and SEQ ID NO: 126 and SEQ ID NO: 127 and SEQ ID NO: 128; or
   (xiv) SEQ ID NO: 129 and SEQ ID NO: 130 and SEQ ID NO: 131 and SEQ ID NO: 132 and SEQ ID NO: 133 and SEQ ID NO: 134; or
   (xv) SEQ ID NO: 135 and SEQ ID NO: 136 and SEQ ID NO: 137 and SEQ ID NO: 138 and SEQ ID NO: 139 and SEQ ID NO: 140; or
   (xvi) SEQ ID NO: 141 and SEQ ID NO: 142 and SEQ ID NO: 143 and SEQ ID NO: 144 and SEQ ID NO: 145 and SEQ ID NO: 146; or
   (xvii) SEQ ID NO: 147 and SEQ ID NO: 148 and SEQ ID NO: 149 and SEQ ID NO: 150 and SEQ ID NO: 151 and SEQ ID NO: 152; or
   (xviii) SEQ ID NO: 153 and SEQ ID NO: 154 and SEQ ID NO: 155 and SEQ ID NO: 156 and SEQ ID NO: 157 and SEQ ID NO: 158; or
   (xix) SEQ ID NO: 159 and SEQ ID NO: 160 and SEQ ID NO: 161 and SEQ ID NO: 162 and SEQ ID NO: 163 and SEQ ID NO: 164; or
   (xx) SEQ ID NO: 165 and SEQ ID NO: 166 and SEQ ID NO: 167 and SEQ ID NO: 168 and SEQ ID NO: 169 and SEQ ID NO: 170; or
   (xxi) SEQ ID NO: 171 and SEQ ID NO: 172 and SEQ ID NO: 173 and SEQ ID NO: 174 and SEQ ID NO: 175 and SEQ ID NO: 176; or
   (xxii) SEQ ID NO: 177 and SEQ ID NO: 178 and SEQ ID NO: 179 and SEQ ID NO: 180 and SEQ ID NO: 181 and SEQ ID NO: 182; or
   (xxiii) SEQ ID NO: 183 and SEQ ID NO: 184 and SEQ ID NO: 185 and SEQ ID NO: 186 and SEQ ID NO: 187 and SEQ ID NO: 188; or
   (xxiv) SEQ ID NO: 189 and SEQ ID NO: 190 and SEQ ID NO: 191 and SEQ ID NO: 192 and SEQ ID NO: 193 and SEQ ID NO: 194.
23. A combination for use, a use, a method, a first antibody molecule for use, a pharmaceutical composition, or a kit according to any one of paragraphs 1-22, wherein the first antibody molecule comprises the following amino acid sequences:
   (i) SEQ ID NO: 3 and SEQ ID NO: 27; or
   (ii) SEQ IS NO: 4 and SEQ ID NO: 28; or
   (iii) SEQ IS NO: 5 and SEQ ID NO: 29; or
   (iv) SEQ ID NO: 6 and SEQ ID NO: 30; or
   (v) SEQ ID NO: 7 and SEQ ID NO: 31; or
   (vi) SEQ ID NO: 8 and SEQ ID NO: 32; or
   (vii) SEQ ID NO: 9 and SEQ ID NO: 33; or
   (viii) SEQ ID NO: 10 and SEQ ID NO: 34; or
   (ix) SEQ ID NO: 11 and SEQ ID NO: 35; or
   (x) SEQ ID NO: 12 and SEQ ID NO: 36; or
   (xi) SEQ ID NO: 13 and SEQ ID NO: 37; or
   (xii) SEQ ID NO: 14 and SEQ ID NO: 38; or
   (xiii) SEQ ID NO: 15 and SEQ ID NO: 39; or
   (xiv) SEQ ID NO: 16 and SEQ ID NO: 40; or
   (xv) SEQ ID NO: 17 and SEQ ID NO: 41; or
   (xvi) SEQ ID NO: 18 and SEQ ID NO: 42; or
   (xvii) SEQ ID NO: 19 and SEQ ID NO: 43; or
   (xviii) SEQ ID NO: 20 and SEQ ID NO: 44; or
   (xix) SEQ ID NO: 21 and SEQ ID NO: 45; or
   (xx) SEQ ID NO: 22 and SEQ ID NO: 46; or
   (xxi) SEQ ID NO: 23 and SEQ ID NO: 47; or
   (xxii) SEQ ID NO: 24 and SEQ ID NO: 48; or
   (xxiii) SEQ ID NO: 25 and SEQ ID NO: 49; or
   (xxiv) SEQ ID NO: 26 and SEQ ID NO: 50.
24. A combination for use, a use, a method, a first antibody molecule for use, a pharmaceutical composition, or a kit according to any one of paragraphs 1-17, wherein the first antibody molecule is an antibody molecule that is capable of competing for binding to FcγRIIb with an antibody molecule according to any one of paragraphs 18-23.
25. A combination for use, a use, a method, or a first antibody molecule for use according to any one of paragraphs 1-5 and 8-24, wherein the cancer is a FcγRIIb-positive B-cell cancer.
26. A combination for use, a use, a method, or a first antibody molecule for use according to any one of paragraphs 1-5 and 8-24, wherein the cancer is a FcγRIIb-negative cancer.
27. A combination for use, a use, a method, or a first antibody molecule for use according to paragraph 26, wherein the FcγRIIb-negative cancer is a solid cancer.
28. A combination for use, a use, a method, or a first antibody molecule for use according to paragraph 27, wherein the solid cancer is selected from the group comprising: a carcinoma, a sarcoma, and a lymphoma.
29. A combination for use, a use, a method, or a first antibody molecule for use according to paragraphs 27 or 28, wherein the solid cancer is selected from the group comprising: melanoma, prostate cancer, colorectal cancer, hepatocellular carcinoma, lung cancer, bladder cancer, kidney cancer, gastric cancer, cervical cancer, Merkel cell carcinoma, or ovarian cancer; and/or wherein the solid cancer is an immune deserted tumour or an immune excluded tumour or a tumour with poor immune infiltration.
30. A combination for use, a use, a method, or a first antibody molecule for use according to any one of paragraphs 1-4 and 8-29, wherein the cancer that is resistant to treatment with an antibody molecule that specifically binds to PD-1 or PD-L1, and/or an antibody molecule that specifically binds to CTLA-4 is relapsed and/or refractory cancer.
31. A combination for use, a use, a method, or a first antibody molecule for use according to any one of paragraphs 1-5 and 8-30, wherein the patient has previously been treated with an antibody molecule that specifically binds to PD-1 or PD-L1, and/or an antibody molecule that specifically binds to CTLA-4, optionally wherein the patient has become resistant following said treatment.
32. A combination for use, a use, a method, or a first antibody molecule for use according to any one of paragraphs 1-5 and 8-30, wherein the patient has not previously been treated with an antibody molecule that specifically binds to PD-1 or PD-L1, and/or an antibody molecule that specifically binds to CTLA-4, optionally wherein the patient is inherently resistant to said treatment.
33. A combination for use, a use, a method, or a first antibody molecule for use, a pharmaceutical composition, or a kit according to any one of paragraphs 1-32, wherein the second antibody molecule and/or the third antibody molecule is selected from the group consisting of a human antibody molecule, a humanized antibody molecule, and an antibody molecule of human origin.
34. A combination for use, a use, a method, or a first antibody molecule for use, a pharmaceutical composition, or a kit according to any one of paragraphs 1-33, wherein the second antibody molecule and/or third antibody molecule is a monoclonal antibody molecule or an antibody molecule of monoclonal origin.
35. A combination for use, a use, a method, or a first antibody molecule for use, a pharmaceutical composition, or a kit according to any one of paragraphs 1-34, wherein the second antibody molecule and/or third antibody molecule is selected from the group consisting of a full-size antibody, a chimeric antibody, a single chain antibody, and an antigen-binding fragment thereof retaining the ability to bind an Fcγ receptor via its Fc region.
36. A combination for use, a use, a method, or a first antibody molecule for use, a pharmaceutical composition, or a kit according to any one of paragraphs 1-35, wherein the second antibody molecule and/or third antibody molecule is a human IgG antibody, a humanized IgG antibody molecule or an IgG antibody molecule of human origin.
37. A combination for use, a use, a method, or a first antibody molecule for use, a pharmaceutical composition, or a kit according to any one of paragraphs 1-36, wherein the second antibody molecule and/or third antibody molecule binds at least one activating Fcγ receptor via its Fc region.
38. A combination for use, a use, a method, or a first antibody molecule for use, a pharmaceutical composition, or a kit according to any one of paragraphs 1-36, wherein the second antibody molecule and/or third antibody molecule has been engineered for improved binding to activating Fc gamma receptors.
39. Use of a first antibody molecule that specifically binds to FcγRIIb via its Fab region, and that lacks an Fc region or has reduced binding to Fcγ receptors via its Fc region, for treating cancer in a patient;
   characterised in that the first antibody molecule reduces and/or prevents resistance in the cancer to treatment with a second antibody molecule that specifically binds to CTLA-4.
40. A use according to paragraph 39, wherein the second antibody molecule is administered at a dose that is lower than the tolerated therapeutic dose.
41. A combination comprising:
   a first antibody molecule that specifically binds to FcγRIIb via its Fab region, and that lacks an Fc region or has reduced binding to Fcγ receptors via its Fc region; and
a second antibody molecule that specifically binds to CTLA-4 and that binds to at least one Fcγ receptor via its Fc region;
for use in treating cancer in a patient,
characterised in that the combination comprises a dose of the second antibody molecule that is lower than the tolerated therapeutic dose.
42. Use of:
a first antibody molecule that specifically binds to FcγRIIb via its Fab region, and that lacks an Fc region or has reduced binding to Fcγ receptors via its Fc region; and
a second antibody molecule that specifically binds to CTLA-4 and that binds to at least one Fcγ receptor via its Fc region;
in the manufacture of a medicament for treating cancer in a patient,
characterised in that the combination comprises a dose of the second antibody molecule that is lower than the tolerated therapeutic dose.
43. A method for treating cancer in an individual, the method comprising administering to the patient:
   a first antibody molecule that specifically binds to FcγRIIb via its Fab region, and that lacks an Fc region or has reduced binding to Fcγ receptors via its Fc region;
   a second antibody molecule that specifically binds to CTLA-4 and that binds to at least one Fcγ receptor via its Fc region;
characterised in that the dose of the second antibody molecule that is administered is lower than the tolerated therapeutic dose.
44. A first antibody molecule that specifically binds to FcγRIIb via its Fab region, and that lacks an Fc region or has reduced binding to Fcγ receptors via its Fc region, for use in combination with:
   a second antibody molecule that specifically binds to CTLA-4 and that binds to at least one Fcγ receptor via its Fc region;
   for treating cancer in a patient, characterised in that the dose of the second antibody molecule that is used is lower than the tolerated therapeutic dose.
45. A pharmaceutical composition comprising:
   a first antibody molecule that specifically binds to FcγRIIb via its Fab region, and that lacks an Fc region or has reduced binding to Fcγ receptors via its Fc region;
   a second antibody molecule that specifically binds to CTLA-4 and that binds to at least one Fcγ receptor via its Fc region,
characterised in that the second antibody molecule is present at a dose which is lower than the tolerated therapeutic dose.
46. A kit comprising:
   a first antibody molecule that specifically binds to FcγRIIb via its Fab region, and that lacks an Fc region or has reduced binding to Fcγ receptors via its Fc region;
   a second antibody molecule that specifically binds to CTLA-4 and that binds to at least one Fcγ receptor via its Fc region,
characterised in that the second antibody molecule is present at a dose that is lower than the tolerated therapeutic dose.
47. A combination for use, a use, a method, a first antibody molecule for use, a pharmaceutical composition, or a kit according to any one of paragraphs 40-46, wherein the dose of the second antibody molecule is lower than the maximum tolerated therapeutic dose.
48. A combination for use, a use, a method, a first antibody molecule for use, a pharmaceutical composition, or a kit according to any one of paragraphs 40-47, wherein the dose of the second antibody molecule is at least 50% lower than the tolerated therapeutic dose.
49. A combination for use, a use, a method, a first antibody molecule for use, a pharmaceutical composition, or a kit according to any one of paragraphs 40-48, wherein the dose of the second antibody molecule is at least 70% lower than the tolerated therapeutic dose.
50. A combination for use, a use, a method, a first antibody molecule for use, a pharmaceutical composition, or a kit according to any one of paragraphs 40-49, wherein the dose of the second antibody molecule is at least 80% lower than the tolerated therapeutic dose.
51. A combination for use, a use, a method, a first antibody molecule for use, a pharmaceutical composition or a kit according to any one of paragraphs 40-50, wherein the dose of the second antibody molecule is lower than the minimum effective therapeutic dose.
52. A combination for use, a use, a method, a first antibody molecule for use, a pharmaceutical composition, or a kit according to any one of paragraphs 40-51, wherein the therapeutic effect of the first antibody molecule and the second antibody molecule used at the lower dose, is comparable to the therapeutic effect of the second antibody molecule in the absence of the first antibody molecule at the maximum tolerated therapeutic dose of the second antibody molecule.
53. A combination for use, a use, a method, a first antibody molecule for use, a pharmaceutical composition, or a kit according to any one of paragraphs 40-52 wherein use of the second antibody molecule at the lower dose improves tolerability of the second antibody molecule.
54. A combination for use, a use, a method, a first antibody molecule for use, a pharmaceutical composition, or a kit according to any one of paragraphs 40-53 wherein the use of the second antibody molecule at the lower dose reduces side effects and/or reduces toxicity in the subject associated with the use of the second antibody molecule.
55. A combination for use, a use, a method, a first antibody molecule for use, a pharmaceutical composition or a kit according to any one of paragraphs 39-54, wherein the second antibody molecule is ipilimumab and/or tremelimumab.
56. A combination for use, a use, a method, a first antibody molecule for use, a pharmaceutical composition, or a kit according to any one of paragraphs 39-56, wherein the dose of the second antibody molecule is lower than 10 mg/kg.
57. A combination for use, a use, a method, a first antibody for use, a pharmaceutical composition, or a kit according to any one of paragraphs 39-56, wherein the dose of the second antibody molecule is 3 mg/kg or lower than 3 mg/kg.
58. A combination for use, a use, a method, a first antibody molecule for use, a pharmaceutical composition, or a kit according to any one of paragraphs 39-57, wherein the dose of the second antibody molecule is 2 mg/kg or lower than 2 mg/kg.
59. A combination for use, a use, a method, a first antibody molecule for use, a pharmaceutical composition, or a kit according to any one of paragraphs 39-58, wherein the dose of the second antibody molecule is 1 mg/kg or lower than 1 mg/kg.
60. A combination for use, a use, a method, a first antibody molecule for use, a pharmaceutical composition, or a kit according to any one of paragraphs 39-59, wherein the use or method does not also involve administration of an antibody molecule that specifically binds PD-1 or PD-L1 and/or the pharmaceutical composition or kit does not also comprise an antibody molecule that specifically binds PD-1 or PD-L1.
61. A combination for use, a use, a method, a first antibody molecule for use, a pharmaceutical composition, or a kit according to any one of paragraphs 39-60 wherein the first antibody molecule lacks an Fc region.
62. A combination for use, a use, a method, a first antibody molecule for use, a pharmaceutical composition, or a kit according to paragraphs 39-60, wherein the first antibody molecule has reduced binding to Fcγ receptors via its Fc region, and has an aglycosylated Fc region.
63. A combination for use, a use, a method, a first antibody molecule for use, a pharmaceutical composition, or a kit according to any one of paragraphs 39-62, wherein the first antibody molecule is selected from the group consisting of a human antibody molecule, a humanized antibody molecule, and an antibody molecule of human origin.
64. A combination for use, a use, a method, a first antibody molecule for use, a pharmaceutical composition, or a kit according to any one of paragraphs 39-63, wherein the first antibody molecule is a monoclonal antibody molecule or an antibody molecule of monoclonal origin.
65. A combination for use, a use, a method, a first antibody molecule for use, a pharmaceutical composition, or a kit according to any one of paragraphs 39-64, wherein the first antibody molecule is selected from the group consisting of: a full-length antibody, a chimeric antibody, a single chain antibody, a Fab fragment, a (Fab′)2 fragment, a Fab′ fragment, a (Fab′)2 fragment, a Fv fragment, and an scFv fragment.
66. A combination for use, a use, a method, a first antibody molecule for use, a pharmaceutical composition, or a kit according to any one of paragraphs 39-65, wherein the first antibody molecule is a human IgG antibody molecule having an aglycosylated Fc region or an IgG antibody molecule of human origin having an aglycosylated Fc region.
67. A combination for use, a use, a method, a first antibody molecule for use, a pharmaceutical composition for use, or a kit for use according to paragraph 66, wherein the IgG antibody molecule is an IgG1 or IgG2 antibody molecule.
68. A combination for use, a use, a method, a first antibody molecule for use, a pharmaceutical composition, or a kit according to paragraph 67, wherein the IgG antibody molecule is an aglycosylated human IgG1 or an aglycosylated humanized murine antibody or an aglycosylated humanized llama hcIgG antibody or a an aglycosylated chimerised murine IgG.
69. A combination for use, a use, a method, a first antibody molecule for use, a pharmaceutical composition, or a kit according to paragraph 68, wherein the first antibody molecule has been aglycosylated through amino acid substitution in position 297.
70. A combination for use, a use, a method, a first antibody molecule for use, a pharmaceutical composition, or a kit according to paragraph 69, wherein the first antibody molecule has been aglycosylated through an N297Q substitution.
71. A combination for use, a use, a method, a first antibody molecule for use, a pharmaceutical composition, or a kit according to any one of paragraphs 39-70, wherein the first antibody molecule comprises a variable heavy chain (VH) comprising the following CDRs:
   (i) SEQ ID NO: 51 and SEQ ID NO: 52 and SEQ ID NO: 53; or
   (ii) SEQ ID NO: 57 and SEQ ID NO: 58 and SEQ ID NO: 59; or
   (iii) SEQ ID NO: 63 and SEQ ID NO: 64 and SEQ ID NO: 65; or
   (iv) SEQ ID NO: 69 and SEQ ID NO: 70 and SEQ ID NO: 71; or
   (v) SEQ ID NO: 75 and SEQ ID NO: 76 and SEQ ID NO: 77; or
   (vi) SEQ ID NO: 81 and SEQ ID NO: 82 and SEQ ID NO: 83; or
   (vii) SEQ ID NO: 87 and SEQ ID NO: 88 and SEQ ID NO: 89; or
   (viii) SEQ ID NO: 93 and SEQ ID NO: 94 and SEQ ID NO: 95; or
   (ix) SEQ ID NO: 99 and SEQ ID NO: 100 and SEQ ID NO: 101; or
   (x) SEQ ID NO: 105 and SEQ ID NO: 106 and SEQ ID NO: 107; or
   (xi) SEQ ID NO: 111 and SEQ ID NO: 112 and SEQ ID NO: 113; or
   (xii) SEQ ID NO: 117 and SEQ ID NO: 118 and SEQ ID NO: 119; or
   (xiii) SEQ ID NO: 123 and SEQ ID NO: 124 and SEQ ID NO: 125; or
   (xiv) SEQ ID NO: 129 and SEQ ID NO: 130 and SEQ ID NO: 131; or
   (xv) SEQ ID NO: 135 and SEQ ID NO: 136 and SEQ ID NO: 137; or
   (xvi) SEQ ID NO: 141 and SEQ ID NO: 142 and SEQ ID NO: 143; or
   (xvii) SEQ ID NO: 147 and SEQ ID NO: 148 and SEQ ID NO: 149; or
   (xviii) SEQ ID NO: 153 and SEQ ID NO: 154 and SEQ ID NO: 155; or
   (xix) SEQ ID NO: 159 and SEQ ID NO: 160 and SEQ ID NO: 161; or
   (xx) SEQ ID NO: 165 and SEQ ID NO: 166 and SEQ ID NO: 167; or
   (xxi) SEQ ID NO: 171 and SEQ ID NO: 172 and SEQ ID NO: 173; or
   (xxii) SEQ ID NO: 177 and SEQ ID NO: 178 and SEQ ID NO: 179; or
   (xxiii) SEQ ID NO: 183 and SEQ ID NO: 184 and SEQ ID NO: 185; or
   (xxiv) SEQ ID NO: 189 and SEQ ID NO: 190 and SEQ ID NO: 191.
72. A combination for use, a use, a method, a first antibody molecule for use, a pharmaceutical composition, or a kit according to any one of paragraphs 39-71, wherein the first antibody molecule comprises a variable light chain (VL) comprising the following CDRs:
   (i) SEQ ID NO: 54 and SEQ ID NO: 55 and SEQ ID NO: 56; or
   (ii) SEQ ID NO: 60 and SEQ ID NO: 61 and SEQ ID NO: 62; or
   (iii) SEQ ID NO: 66 and SEQ ID NO: 67 and SEQ ID NO: 68; or
   (iv) SEQ ID NO: 72 and SEQ ID NO: 73 and SEQ ID NO: 74; or
   (v) SEQ ID NO: 78 and SEQ ID NO: 79 and SEQ ID NO: 80; or
   (vi) SEQ ID NO: 84 and SEQ ID NO: 85 and SEQ ID NO: 86; or
   (vii) SEQ ID NO: 90 and SEQ ID NO: 91 and SEQ ID NO: 92; or
   (viii) SEQ ID NO: 96 and SEQ ID NO: 97 and SEQ ID NO: 98; or
   (ix) SEQ ID NO: 102 and SEQ ID NO: 103 and SEQ ID NO: 104; or
   (x) SEQ ID NO: 108 and SEQ ID NO: 109 and SEQ ID NO: 110; or
   (xi) SEQ ID NO: 114 and SEQ ID NO: 115 and SEQ ID NO: 116; or
   (xii) SEQ ID NO: 120 and SEQ ID NO: 121 and SEQ ID NO: 122; or
   (xiii) SEQ ID NO: 126 and SEQ ID NO: 127 and SEQ ID NO: 128; or
   (xiv) SEQ ID NO: 132 and SEQ ID NO: 133 and SEQ ID NO: 134; or
   (xv) SEQ ID NO: 138 and SEQ ID NO: 139 and SEQ ID NO: 140; or
   (xvi) SEQ ID NO: 144 and SEQ ID NO: 145 and SEQ ID NO: 146; or
   (xvii) SEQ ID NO: 150 and SEQ ID NO: 151 and SEQ ID NO: 152; or
   (xviii) SEQ ID NO: 156 and SEQ ID NO: 157 and SEQ ID NO: 158; or
   (xix) SEQ ID NO: 162 and SEQ ID NO: 163 and SEQ ID NO: 164; or
   (xx) SEQ ID NO: 168 and SEQ ID NO: 169 and SEQ ID NO: 170; or
   (xxi) SEQ ID NO: 174 and SEQ ID NO: 175 and SEQ ID NO: 176; or
   (xxii) SEQ ID NO: 180 and SEQ ID NO: 181 and SEQ ID NO: 182; or
   (xxiii) SEQ ID NO: 186 and SEQ ID NO: 187 and SEQ ID NO: 188; or
   (xxiv) SEQ ID NO: 192 and SEQ ID NO: 193 and SEQ ID NO: 194.
73. A combination for use, a use, a method, a first antibody molecule for use, a pharmaceutical composition, or a kit according to any one of paragraphs 39-72, wherein the first antibody molecule comprises a variable heavy chain (VH) amino acid sequence selected from the group consisting of: SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; and SEQ ID NO: 26.
74. A combination for use, a use, a method, a first antibody molecule for use, a pharmaceutical composition, or a kit according to any one of claims 39-73, wherein the first antibody molecule comprises a variable light chain (VL) amino acid sequence selected from the group consisting of: SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 38; SEQ ID NO: 39; SEQ ID NO: 40; SEQ ID NO: 41; SEQ ID NO: 42; SEQ ID NO: 43; SEQ ID NO: 44; SEQ ID NO: 45; SEQ ID NO: 46; SEQ ID NO: 47; SEQ ID NO: 48; SEQ ID NO: 49; and SEQ ID NO: 50.
75. A combination for use, a use, a method, a first antibody molecule for use, a pharmaceutical composition, or a kit according to any one of paragraphs 39-74, wherein the first antibody molecule comprises the following CDR amino acid sequences:
   (i) SEQ ID NO: 51 and SEQ ID NO: 52 and SEQ ID NO: 53 and SEQ ID NO: 54 and SEQ ID NO: 55 and SEQ ID NO: 56; or
   (ii) SEQ ID NO: 57 and SEQ ID NO: 58 and SEQ ID NO: 59 and SEQ ID NO: 60 and SEQ ID NO: 61 and SEQ ID NO: 62; or
   (iii) SEQ ID NO: 63 and SEQ ID NO: 64 and SEQ ID NO: 65 and SEQ ID NO: 66 and SEQ ID NO: 67 and SEQ ID NO: 68; or
   (iv) SEQ ID NO: 69 and SEQ ID NO: 70 and SEQ ID NO: 71 and SEQ ID NO: 72 and SEQ ID NO: 73 and SEQ ID NO: 74; or
   (v) SEQ ID NO: 75 and SEQ ID NO: 76 and SEQ ID NO: 77 and SEQ ID NO: 78 and SEQ ID NO: 79 and SEQ ID NO: 80; or
   (vi) SEQ ID NO: 81 and SEQ ID NO: 82 and SEQ ID NO: 83 and SEQ ID NO: 84 and SEQ ID NO: 85 and SEQ ID NO: 86; or
   (vii) SEQ ID NO: 87 and SEQ ID NO: 88 and SEQ ID NO: 89 and SEQ ID NO: 90 and SEQ ID NO: 91 and SEQ ID NO: 92; or
   (viii) SEQ ID NO: 93 and SEQ ID NO: 94 and SEQ ID NO: 95 and SEQ ID NO: 96 and SEQ ID NO: 97 and SEQ ID NO: 98; or
   (ix) SEQ ID NO: 99 and SEQ ID NO: 100 and SEQ ID NO: 101 and SEQ ID NO: 102 and SEQ ID NO: 103 and SEQ ID NO: 104; or
   (x) SEQ ID NO: 105 and SEQ ID NO: 106 and SEQ ID NO: 107 and SEQ ID NO: 108 and SEQ ID NO: 109 and SEQ ID NO: 110; or
   (xi) SEQ ID NO: 111 and SEQ ID NO: 112 and SEQ ID NO: 113 and SEQ ID NO: 114 and SEQ ID NO: 115 and SEQ ID NO: 116; or
   (xii) SEQ ID NO: 117 and SEQ ID NO: 118 and SEQ ID NO: 119 and SEQ ID NO: 120 and SEQ ID NO: 121 and SEQ ID NO: 122; or
   (xiii) SEQ ID NO: 123 and SEQ ID NO: 124 and SEQ ID NO: 125 and SEQ ID NO: 126 and SEQ ID NO: 127 and SEQ ID NO: 128; or
   (xiv) SEQ ID NO: 129 and SEQ ID NO: 130 and SEQ ID NO: 131 and SEQ ID NO: 132 and SEQ ID NO: 133 and SEQ ID NO: 134; or
   (xv) SEQ ID NO: 135 and SEQ ID NO: 136 and SEQ ID NO: 137 and SEQ ID NO: 138 and SEQ ID NO: 139 and SEQ ID NO: 140; or
   (xvi) SEQ ID NO: 141 and SEQ ID NO: 142 and SEQ ID NO: 143 and SEQ ID NO: 144 and SEQ ID NO: 145 and SEQ ID NO: 146; or
   (xvii) SEQ ID NO: 147 and SEQ ID NO: 148 and SEQ ID NO: 149 and SEQ ID NO: 150 and SEQ ID NO: 151 and SEQ ID NO: 152; or
   (xviii) SEQ ID NO: 153 and SEQ ID NO: 154 and SEQ ID NO: 155 and SEQ ID NO: 156 and SEQ ID NO: 157 and SEQ ID NO: 158; or
   (xix) SEQ ID NO: 159 and SEQ ID NO: 160 and SEQ ID NO: 161 and SEQ ID NO: 162 and SEQ ID NO: 163 and SEQ ID NO: 164; or
   (xx) SEQ ID NO: 165 and SEQ ID NO: 166 and SEQ ID NO: 167 and SEQ ID NO: 168 and SEQ ID NO: 169 and SEQ ID NO: 170; or
   (xxi) SEQ ID NO: 171 and SEQ ID NO: 172 and SEQ ID NO: 173 and SEQ ID NO: 174 and SEQ ID NO: 175 and SEQ ID NO: 176; or
   (xxii) SEQ ID NO: 177 and SEQ ID NO: 178 and SEQ ID NO: 179 and SEQ ID NO: 180 and SEQ ID NO: 181 and SEQ ID NO: 182; or
   (xxiii) SEQ ID NO: 183 and SEQ ID NO: 184 and SEQ ID NO: 185 and SEQ ID NO: 186 and SEQ ID NO: 187 and SEQ ID NO: 188; or
   (xxiv) SEQ ID NO: 189 and SEQ ID NO: 190 and SEQ ID NO: 191 and SEQ ID NO: 192 and SEQ ID NO: 193 and SEQ ID NO: 194.
76. A combination for use, a use, a method, a first antibody molecule for use, a pharmaceutical composition, or a kit according to any one of paragraphs 39-75, wherein the first antibody molecule comprises the following amino acid sequences:
   (i) SEQ ID NO: 3 and SEQ ID NO: 27; or
   (ii) SEQ IS NO: 4 and SEQ ID NO: 28; or
   (iii) SEQ IS NO: 5 and SEQ ID NO: 29; or
   (iv) SEQ ID NO: 6 and SEQ ID NO: 30; or
   (v) SEQ ID NO: 7 and SEQ ID NO: 31; or
   (vi) SEQ ID NO: 8 and SEQ ID NO: 32; or
   (vii) SEQ ID NO: 9 and SEQ ID NO: 33; or
   (viii) SEQ ID NO: 10 and SEQ ID NO: 34; or
   (ix) SEQ ID NO: 11 and SEQ ID NO: 35; or
   (x) SEQ ID NO: 12 and SEQ ID NO: 36; or
   (xi) SEQ ID NO: 13 and SEQ ID NO: 37; or
   (xii) SEQ ID NO: 14 and SEQ ID NO: 38; or
   (xiii) SEQ ID NO: 15 and SEQ ID NO: 39; or
   (xiv) SEQ ID NO: 16 and SEQ ID NO: 40; or
   (xv) SEQ ID NO: 17 and SEQ ID NO: 41; or
   (xvi) SEQ ID NO: 18 and SEQ ID NO: 42; or
   (xvii) SEQ ID NO: 19 and SEQ ID NO: 43; or
   (xviii) SEQ ID NO: 20 and SEQ ID NO: 44; or
   (xix) SEQ ID NO: 21 and SEQ ID NO: 45; or
   (xx) SEQ ID NO: 22 and SEQ ID NO: 46; or
   (xxi) SEQ ID NO: 23 and SEQ ID NO: 47; or
   (xxii) SEQ ID NO: 24 and SEQ ID NO: 48; or
   (xxiii) SEQ ID NO: 25 and SEQ ID NO: 49; or
   (xxiv) SEQ ID NO: 26 and SEQ ID NO: 50.
77. A combination for use, a use, a method, a first antibody molecule for use, a pharmaceutical composition, or a kit according to any one of paragraphs 39-70, wherein the first antibody molecule is an antibody molecule that is capable of competing for binding to FcγRIIb with an antibody molecule according to any one of paragraphs 71-76.
78. A combination for use, a use, a method, or a first antibody molecule for use according to any one of paragraphs 39-44 or 47-77, wherein the cancer is a FcγRIIb-positive B-cell cancer.
79. A combination for use, a use, a method, or a first antibody molecule for use according to any one of paragraphs 39-44 or 47-77, wherein the cancer is a FcγRIIb-negative cancer.
80. A combination for use, a use, a method, or a first antibody molecule for use according to paragraph 79, wherein the FcγRIIb-negative cancer is a solid cancer.
81. A combination for use, a use, a method, or a first antibody molecule for use according to paragraph 80, wherein the solid cancer is selected from the group comprising: a carcinoma, a sarcoma, and a lymphoma.
82. A combination for use, a use, a method, or a first antibody molecule for use according to paragraphs 80 or 81, wherein the solid cancer is selected from the group comprising: melanoma, pancreatic cancer, breast cancer, prostate cancer, colorectal cancer, lung cancer, bladder cancer, kidney cancer, mesothelioma, Merkel cell carcinoma, gastric cancer, cervical cancer, ovarian cancer, and head and neck cancer.
83. A combination for use, a use, a method, a first antibody molecule for use, a pharmaceutical composition, or a kit substantially as claimed herein with reference to the accompanying numbered paragraphs, claims, description, examples and figures.

1. A combination comprising:
a first antibody molecule that specifically binds to FcγRIIb via its Fab region, and that lacks an Fc region or has reduced binding to Fcγ receptors via its Fc region;
a second antibody molecule that specifically binds to PD-1 or PD-L1; and
a third antibody molecule that specifically binds to CTLA-4 and that binds to at least one Fcγ receptor via its Fc region;
for use in treating cancer in a patient, wherein the cancer is resistant to treatment with an antibody molecule that specifically binds to PD-1 or PD-L1, and/or an antibody molecule that specifically binds to CTLA-4.

2. (canceled)

3. A method for treating cancer in a patient, the method comprising administering to the patient:
a first antibody molecule that specifically binds to FcγRIIb via its Fab region, and that lacks an Fc region or has reduced binding to Fcγ receptors via its Fc region;
a second antibody molecule that specifically binds to PD-1 or PD-L1; and
a third antibody molecule that specifically binds to CTLA-4 and that binds to at least one Fcγ receptor via its Fc region;
wherein the cancer is resistant to treatment with an antibody molecule that specifically binds to PD-1 or PD-L1, and/or an antibody molecule that specifically binds to CTLA-4.

4. (canceled)

5. (canceled)

6. A pharmaceutical composition comprising:
a first antibody molecule that specifically binds to FcγRIIb via its Fab region, and that lacks an Fc region or has reduced binding to Fcγ receptors via its Fc region;
a second antibody molecule that specifically binds to PD-1 or PD-L1; and
a third antibody molecule that specifically binds to CTLA-4. and that binds to at least one Fcγ receptor via its Fc region.

7. A kit comprising:
a first antibody molecule that specifically binds to FcγRIIb via its Fab region, and that lacks an Fc region or has reduced binding to Fcγ receptors via its Fc region;
a second antibody molecule that specifically binds to PD-1 or PD-L1; and
a third antibody molecule that specifically binds to CTLA-4 and that binds to at least one Fcγ receptor via its Fc region.

8. A combination for use, a use, a method, a first antibody molecule for use, a pharmaceutical composition, or a kit according to claim 1, wherein the first antibody molecule lacks the Fc region or has an aglycosylated Fc region.

9. A combination for use, a use, a method, a first antibody molecule for use, a pharmaceutical composition, or a kit according to claim 1, wherein the first antibody molecule is selected from the group consisting of a human antibody molecule, a humanized antibody molecule, and an antibody molecule of human origin.

10. A combination for use, a use, a method, a first antibody for use, a pharmaceutical composition, or a kit according to claim 1, wherein the first antibody molecule comprises a variable heavy chain (VH) comprising the following CDRs:
(i) SEQ ID NO: 51 and SEQ ID NO: 52 and SEQ ID NO: 53; or
(ii) SEQ ID NO: 57 and SEQ ID NO: 58 and SEQ ID NO: 59; or
(iii) SEQ ID NO: 63 and SEQ ID NO: 64 and SEQ ID NO: 65; or
(iv) SEQ ID NO: 69 and SEQ ID NO: 70 and SEQ ID NO: 71; or
(v) SEQ ID NO: 75 and SEQ ID NO: 76 and SEQ ID NO: 77; or
(vi) SEQ ID NO: 81 and SEQ ID NO: 82 and SEQ ID NO: 83; or
(vii) SEQ ID NO: 87 and SEQ ID NO: 88 and SEQ ID NO: 89; or
(viii) SEQ ID NO: 93 and SEQ ID NO: 94 and SEQ ID NO: 95; or
(ix) SEQ ID NO: 99 and SEQ ID NO: 100 and SEQ ID NO: 101; or
(x) SEQ ID NO: 105 and SEQ ID NO: 106 and SEQ ID NO: 107; or
(xi) SEQ ID NO: 111 and SEQ ID NO: 112 and SEQ ID NO: 113; or
(xii) SEQ ID NO: 117 and SEQ ID NO: 118 and SEQ ID NO: 119; or
(xiii) SEQ ID NO: 123 and SEQ ID NO: 124 and SEQ ID NO: 125; or
(xiv) SEQ ID NO: 129 and SEQ ID NO: 130 and SEQ ID NO: 131; or
(xv) SEQ ID NO: 135 and SEQ ID NO: 136 and SEQ ID NO: 137; or
(xvi) SEQ ID NO: 141 and SEQ ID NO: 142 and SEQ ID NO: 143; or
(xvii) SEQ ID NO: 147 and SEQ ID NO: 148 and SEQ ID NO: 149; or
(xviii) SEQ ID NO: 153 and SEQ ID NO: 154 and SEQ ID NO: 155; or
(xix) SEQ ID NO: 159 and SEQ ID NO: 160 and SEQ ID NO: 161; or
(xx) SEQ ID NO: 165 and SEQ ID NO: 166 and SEQ ID NO: 167; or
(xxi) SEQ ID NO: 171 and SEQ ID NO: 172 and SEQ ID NO: 173; or
(xxii) SEQ ID NO: 177 and SEQ ID NO: 178 and SEQ ID NO: 179; or
(xxiii) SEQ ID NO: 183 and SEQ ID NO: 184 and SEQ ID NO: 185; or
(xxiv) SEQ ID NO: 189 and SEQ ID NO: 190 and SEQ ID NO: 191.

11. A combination for use, a use, a method, a first antibody molecule for use, a pharmaceutical composition, or a kit according to any one of claims 1-10, wherein the first antibody molecule comprises a variable light chain (VL) comprising the following CDRs:
(i) SEQ ID NO: 54 and SEQ ID NO: 55 and SEQ ID NO: 56; or
(ii) SEQ ID NO: 60 and SEQ ID NO: 61 and SEQ ID NO: 62; or
(iii) SEQ ID NO: 66 and SEQ ID NO: 67 and SEQ ID NO: 68; or
(iv) SEQ ID NO: 72 and SEQ ID NO: 73 and SEQ ID NO: 74; or
(v) SEQ ID NO: 78 and SEQ ID NO: 79 and SEQ ID NO: 80; or
(vi) SEQ ID NO: 84 and SEQ ID NO: 85 and SEQ ID NO: 86; or
(vii) SEQ ID NO: 90 and SEQ ID NO: 91 and SEQ ID NO: 92; or
(viii) SEQ ID NO: 96 and SEQ ID NO: 97 and SEQ ID NO: 98; or
(ix) SEQ ID NO: 102 and SEQ ID NO: 103 and SEQ ID NO: 104; or
(x) SEQ ID NO: 108 and SEQ ID NO: 109 and SEQ ID NO: 110; or
(xi) SEQ ID NO: 114 and SEQ ID NO: 115 and SEQ ID NO: 116; or
(xii) SEQ ID NO: 120 and SEQ ID NO: 121 and SEQ ID NO: 122; or
(xiii) SEQ ID NO: 126 and SEQ ID NO: 127 and SEQ ID NO: 128; or
(xiv) SEQ ID NO: 132 and SEQ ID NO: 133 and SEQ ID NO: 134; or
(xv) SEQ ID NO: 138 and SEQ ID NO: 139 and SEQ ID NO: 140; or
(xvi) SEQ ID NO: 144 and SEQ ID NO: 145 and SEQ ID NO: 146; or
(xvii) SEQ ID NO: 150 and SEQ ID NO: 151 and SEQ ID NO: 152; or
(xviii) SEQ ID NO: 156 and SEQ ID NO: 157 and SEQ ID NO: 158; or
(xix) SEQ ID NO: 162 and SEQ ID NO: 163 and SEQ ID NO: 164; or
(xx) SEQ ID NO: 168 and SEQ ID NO: 169 and SEQ ID NO: 170; or
(xxi) SEQ ID NO: 174 and SEQ ID NO: 175 and SEQ ID NO: 176; or
(xxii) SEQ ID NO: 180 and SEQ ID NO: 181 and SEQ ID NO: 182; or
(xxiii) SEQ ID NO: 186 and SEQ ID NO: 187 and SEQ ID NO: 188; or
(xxiv) SEQ ID NO: 192 and SEQ ID NO: 193 and SEQ ID NO: 194.

12. A combination for use, a use, a method, a first antibody molecule for use, a pharmaceutical composition, or a kit according to any one of claims 1-4410, wherein the first antibody molecule comprises a variable heavy chain (VH) amino acid sequence selected from the group consisting of: SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; and SEQ ID NO: 26.

13. A combination for use, a use, a method, a first antibody molecule for use, a pharmaceutical composition, or a kit according to any one of claims 1-10, wherein the first antibody molecule comprises a variable light chain (VL) amino acid sequence selected from the group consisting of: SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 38; SEQ ID NO: 39; SEQ ID NO: 40; SEQ ID NO: 41; SEQ ID NO: 42; SEQ ID NO: 43; SEQ ID NO: 44; SEQ ID NO: 45; SEQ ID NO: 46; SEQ ID NO: 47; SEQ ID NO: 48; SEQ ID NO: 49; and SEQ ID NO: 50.

14. A combination for use, a use, a method, a first antibody molecule for use, a pharmaceutical composition, or a kit according to claim 1, wherein the first antibody molecule comprises the following CDR amino acid sequences:
(i) SEQ ID NO: 51 and SEQ ID NO: 52 and SEQ ID NO: 53 and SEQ ID NO: 54 and SEQ ID NO: 55 and SEQ ID NO: 56; or
(ii) SEQ ID NO: 57 and SEQ ID NO: 58 and SEQ ID NO: 59 and SEQ ID NO: 60 and SEQ ID NO: 61 and SEQ ID NO: 62; or
(iii) SEQ ID NO: 63 and SEQ ID NO: 64 and SEQ ID NO: 65 and SEQ ID NO: 66 and SEQ ID NO: 67 and SEQ ID NO: 68; or
(iv) SEQ ID NO: 69 and SEQ ID NO: 70 and SEQ ID NO: 71 and SEQ ID NO: 72 and SEQ ID NO: 73 and SEQ ID NO: 74; or
(v) SEQ ID NO: 75 and SEQ ID NO: 76 and SEQ ID NO: 77 and SEQ ID NO: 78 and SEQ ID NO: 79 and SEQ ID NO: 80; or
(vi) SEQ ID NO: 81 and SEQ ID NO: 82 and SEQ ID NO: 83 and SEQ ID NO: 84 and SEQ ID NO: 85 and SEQ ID NO: 86; or
(vii) SEQ ID NO: 87 and SEQ ID NO: 88 and SEQ ID NO: 89 and SEQ ID NO: 90 and SEQ ID NO: 91 and SEQ ID NO: 92; or
(viii) SEQ ID NO: 93 and SEQ ID NO: 94 and SEQ ID NO: 95 and SEQ ID NO: 96 and SEQ ID NO: 97 and SEQ ID NO: 98; or
(ix) SEQ ID NO: 99 and SEQ ID NO: 100 and SEQ ID NO: 101 and SEQ ID NO: 102 and SEQ ID NO: 103 and SEQ ID NO: 104; or
(x) SEQ ID NO: 105 and SEQ ID NO: 106 and SEQ ID NO: 107 and SEQ ID NO: 108 and SEQ ID NO: 109 and SEQ ID NO: 110; or
(xi) SEQ ID NO: 111 and SEQ ID NO: 112 and SEQ ID NO: 113 and SEQ ID NO: 114 and SEQ ID NO: 115 and SEQ ID NO: 116; or
(xii) SEQ ID NO: 117 and SEQ ID NO: 118 and SEQ ID NO: 119 and SEQ ID NO: 120 and SEQ ID NO: 121 and SEQ ID NO: 122; or
(xiii) SEQ ID NO: 123 and SEQ ID NO: 124 and SEQ ID NO: 125 and SEQ ID NO: 126 and SEQ ID NO: 127 and SEQ ID NO: 128; or
(xiv) SEQ ID NO: 129 and SEQ ID NO: 130 and SEQ ID NO: 131 and SEQ ID NO: 132 and SEQ ID NO: 133 and SEQ ID NO: 134; or
(xv) SEQ ID NO: 135 and SEQ ID NO: 136 and SEQ ID NO: 137 and SEQ ID NO: 138 and SEQ ID NO: 139 and SEQ ID NO: 140; or
(xvi) SEQ ID NO: 141 and SEQ ID NO: 142 and SEQ ID NO: 143 and SEQ ID NO: 144 and SEQ ID NO: 145 and SEQ ID NO: 146; or
(xvii) SEQ ID NO: 147 and SEQ ID NO: 148 and SEQ ID NO: 149 and SEQ ID NO: 150 and SEQ ID NO: 151 and SEQ ID NO: 152; or
(xviii) SEQ ID NO: 153 and SEQ ID NO: 154 and SEQ ID NO: 155 and SEQ ID NO: 156 and SEQ ID NO: 157 and SEQ ID NO: 158; or
(xix) SEQ ID NO: 159 and SEQ ID NO: 160 and SEQ ID NO: 161 and SEQ ID NO: 162 and SEQ ID NO: 163 and SEQ ID NO: 164; or
(xx) SEQ ID NO: 165 and SEQ ID NO: 166 and SEQ ID NO: 167 and SEQ ID NO: 168 and SEQ ID NO: 169 and SEQ ID NO: 170; or
(xxi) SEQ ID NO: 171 and SEQ ID NO: 172 and SEQ ID NO: 173 and SEQ ID NO: 174 and SEQ ID NO: 175 and SEQ ID NO: 176; or
(xxii) SEQ ID NO: 177 and SEQ ID NO: 178 and SEQ ID NO: 179 and SEQ ID NO: 180 and SEQ ID NO: 181 and SEQ ID NO: 182; or
(xxiii) SEQ ID NO: 183 and SEQ ID NO: 184 and SEQ ID NO: 185 and SEQ ID NO: 186 and SEQ ID NO: 187 and SEQ ID NO: 188; or
(xxiv) SEQ ID NO: 189 and SEQ ID NO: 190 and SEQ ID NO: 191 and SEQ ID NO: 192 and SEQ ID NO: 193 and SEQ ID NO: 194.

15. A combination for use, a use, a method, a first antibody molecule for use, a pharmaceutical composition, or a kit according to claim 1, wherein the first antibody molecule comprises the following amino acid sequences:
(i) SEQ ID NO: 3 and SEQ ID NO: 27; or
(ii) SEQ IS NO: 4 and SEQ ID NO: 28; or
(iii) SEQ IS NO: 5 and SEQ ID NO: 29; or
(iv) SEQ ID NO: 6 and SEQ ID NO: 30; or
(v) SEQ ID NO: 7 and SEQ ID NO: 31; or
(vi) SEQ ID NO: 8 and SEQ ID NO: 32; or
(vii) SEQ ID NO: 9 and SEQ ID NO: 33; or
(viii) SEQ ID NO: 10 and SEQ ID NO: 34; or
(ix) SEQ ID NO: 11 and SEQ ID NO: 35; or
(x) SEQ ID NO: 12 and SEQ ID NO: 36; or
(xi) SEQ ID NO: 13 and SEQ ID NO: 37; or
(xii) SEQ ID NO: 14 and SEQ ID NO: 38; or
(xiii) SEQ ID NO: 15 and SEQ ID NO: 39; or
(xiv) SEQ ID NO: 16 and SEQ ID NO: 40; or
(xv) SEQ ID NO: 17 and SEQ ID NO: 41; or
(xvi) SEQ ID NO: 18 and SEQ ID NO: 42; or
(xvii) SEQ ID NO: 19 and SEQ ID NO: 43; or
(xviii) SEQ ID NO: 20 and SEQ ID NO: 44; or
(xix) SEQ ID NO: 21 and SEQ ID NO: 45; or
(xx) SEQ ID NO: 22 and SEQ ID NO: 46; or
(xxi) SEQ ID NO: 23 and SEQ ID NO: 47; or
(xxii) SEQ ID NO: 24 and SEQ ID NO: 48; or
(xxiii) SEQ ID NO: 25 and SEQ ID NO: 49; or
(xxiv) SEQ ID NO: 26 and SEQ ID NO: 50.

16. A combination for use, a use, a method, or a first antibody molecule for use according to claim 1, wherein the cancer is a FcγRIIb-positive B-cell cancer or is a FcγRIIb-negative cancer.

17. A combination for use, a use, a method, or a first antibody molecule for use according to claim 16, wherein the FcγRIIb-negative cancer is a solid cancer.

18. A combination for use, a use, a method, or a first antibody molecule for use according to claim 1, wherein the cancer that is resistant to treatment with n antibody molecule that specifically binds to PD-1 or PD-L1, and/or an antibody molecule that specifically binds to CTLA-4 is relapsed and/or refractory cancer.

19. A combination for use, a use, a method, or a first antibody molecule for use, a pharmaceutical composition, or a kit according to claim 1, wherein the second antibody molecule and/or the third antibody molecule is selected from the group consisting of a human antibody molecule, a humanized antibody molecule, and an antibody molecule of human origin.

20. (canceled)

21. (canceled)

22. A combination comprising:
a first antibody molecule that specifically binds to FcγRIIb via its Fab region, and that lacks an Fc region or has reduced binding to Fcγ receptors via its Fc region; and
a second antibody molecule that specifically binds to CTLA-4 and that binds to at least one Fcγ receptor via its Fc region;
for use in treating cancer in a patient,
wherein the combination comprises a dose of the second antibody molecule that is lower than the tolerated therapeutic dose.

23. (canceled)

24. Amethod for treating cancer in an individual, the method comprising administering to the patient:
a first antibody molecule that specifically binds to FcγRIIb via its Fab region, and that lacks an Fc region or has reduced binding to Fcγ receptors via its Fc region;
a second antibody molecule that specifically binds to CTLA-4 and that binds to at least one Fcγ receptor via its Fc region;
wherein the dose of the second antibody molecule that is administered is lower than the tolerated therapeutic dose.

25. (canceled)

26. A pharmaceutical composition comprising:
a first antibody molecule that specifically binds to FcγRIIb via its Fab region, and that lacks an Fc region or has reduced binding to Fcγ receptors via its Fc region;
a second antibody molecule that specifically binds to CTLA-4 and that binds to at least one Fcγ receptor via its Fc region,
wherein the second antibody molecule is present at a dose which is lower than the tolerated therapeutic dose.

27. A kit comprising:
a first antibody molecule that specifically binds to FcγRIIb via its Fab region, and that lacks an Fc region or has reduced binding to Fcγ receptors via its Fc region;
a second antibody molecule that specifically binds to CTLA-4 and that binds to at least one Fcγ receptor via its Fc region,
wherein the second antibody molecule is present at a dose that is lower than the tolerated therapeutic dose.

28. A combination for use, a use, a method, a first antibody molecule for use, a pharmaceutical composition, or a kit according to claim 22, wherein the dose of the second antibody molecule is lower than the maximum tolerated therapeutic dose.

29. A combination for use, a use, a method, a first antibody molecule for use, a pharmaceutical composition or a kit according to claim 22, wherein the dose of the second antibody molecule is lower than the minimum effective therapeutic dose.

30. A combination for use, a use, a method, a first antibody molecule for use, a pharmaceutical composition, or a kit according to claim 22, wherein the therapeutic effect of the first antibody molecule and the second antibody molecule used at the lower dose, is comparable to the therapeutic effect of the second antibody molecule in the absence of the first antibody molecule at the maximum tolerated therapeutic dose of the second antibody molecule.

31. A combination for use, a use, a method, a first antibody molecule for use, a pharmaceutical composition, or a kit according to claim 22 wherein use of the second antibody molecule at the lower dose either (i) improves tolerability of the second antibody molecule, or (ii) reduces side effects and/or reduces toxicity in the subject associated with the use of the second antibody molecule.

32. (canceled)

33. A combination for use, a use, a method, a first antibody molecule for use, a pharmaceutical composition or a kit according to claim 22, wherein the second antibody molecule is ipilimumab and/or tremelimumab.

34. A combination for use, a use, a method, a first antibody molecule for use, a pharmaceutical composition, or a kit according to claim 22, wherein the dose of the second antibody molecule is lower than 10 mg/kg.

35. A combination for use, a use, a method, a first antibody molecule for use, a pharmaceutical composition, or a kit according to claim 22 wherein the first antibody molecule either (i) lacks the Fc region, or (ii) has reduced binding to Fcγ receptors via its Fc region, and has an aglycosylated Fc region.

36. (canceled)

37. A combination for use, a use, a method, a first antibody molecule for use, a pharmaceutical composition, or a kit according to claim 22, wherein the first antibody molecule is selected from the group consisting of a human antibody molecule, a humanized antibody molecule, and an antibody molecule of human origin.

38. A combination for use, a use, a method, a first antibody molecule for use, a pharmaceutical composition, or a kit according to claim 22, wherein the first antibody molecule comprises a variable heavy chain (VH) comprising the following CDRs:
(i) SEQ ID NO: 51 and SEQ ID NO: 52 and SEQ ID NO: 53; or
(ii) SEQ ID NO: 57 and SEQ ID NO: 58 and SEQ ID NO: 59; or
(iii) SEQ ID NO: 63 and SEQ ID NO: 64 and SEQ ID NO: 65; or
(iv) SEQ ID NO: 69 and SEQ ID NO: 70 and SEQ ID NO: 71; or
(v) SEQ ID NO: 75 and SEQ ID NO: 76 and SEQ ID NO: 77; or
(vi) SEQ ID NO: 81 and SEQ ID NO: 82 and SEQ ID NO: 83; or
(vii) SEQ ID NO: 87 and SEQ ID NO: 88 and SEQ ID NO: 89; or
(viii) SEQ ID NO: 93 and SEQ ID NO: 94 and SEQ ID NO: 95; or
(ix) SEQ ID NO: 99 and SEQ ID NO: 100 and SEQ ID NO: 101; or
(x) SEQ ID NO: 105 and SEQ ID NO: 106 and SEQ ID NO: 107; or
(xi) SEQ ID NO: 111 and SEQ ID NO: 112 and SEQ ID NO: 113; or
(xii) SEQ ID NO: 117 and SEQ ID NO: 118 and SEQ ID NO: 119; or
(xiii) SEQ ID NO: 123 and SEQ ID NO: 124 and SEQ ID NO: 125; or
(xiv) SEQ ID NO: 129 and SEQ ID NO: 130 and SEQ ID NO: 131; or
(xv) SEQ ID NO: 135 and SEQ ID NO: 136 and SEQ ID NO: 137; or
(xvi) SEQ ID NO: 141 and SEQ ID NO: 142 and SEQ ID NO: 143; or
(xvii) SEQ ID NO: 147 and SEQ ID NO: 148 and SEQ ID NO: 149; or
(xviii) SEQ ID NO: 153 and SEQ ID NO: 154 and SEQ ID NO: 155; or
(xix) SEQ ID NO: 159 and SEQ ID NO: 160 and SEQ ID NO: 161; or
(xx) SEQ ID NO: 165 and SEQ ID NO: 166 and SEQ ID NO: 167; or
(xxi) SEQ ID NO: 171 and SEQ ID NO: 172 and SEQ ID NO: 173; or
(xxii) SEQ ID NO: 177 and SEQ ID NO: 178 and SEQ ID NO: 179; or
(xxiii) SEQ ID NO: 183 and SEQ ID NO: 184 and SEQ ID NO: 185; or
(xxiv) SEQ ID NO: 189 and SEQ ID NO: 190 and SEQ ID NO: 191.

39. A combination for use, a use, a method, or a first antibody molecule for use according to claim 22, wherein the cancer is a FcγRIIb-positive B-cell cancer or is a FcγRIIb-negative cancer.

40. A combination for use, a use, a method, or a first antibody molecule for use according to claim 17, wherein the solid cancer is selected from the group consisting of: a carcinoma, a sarcoma, a lymphoma, melanoma, prostate cancer, colorectal cancer, hepatocellular carcinoma, lung cancer, bladder cancer, kidney cancer, gastric cancer, cervical cancer, Merkel cell carcinoma, or ovarian cancer; and/or wherein the solid cancer is an immune deserted tumour or an immune excluded tumour or a tumour with poor immune infiltration.

41. A combination for use, a use, a method, a first antibody molecule for use, a pharmaceutical composition, or a kit according to claim 11, wherein the first antibody molecule comprises a variable heavy chain (VH) amino acid sequence selected from the group consisting of: SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; and SEQ ID NO: 26.

42. A combination for use, a use, a method, a first antibody molecule for use, a pharmaceutical composition, or a kit according to claim 41, wherein the first antibody molecule comprises a variable light chain (VL) amino acid sequence selected from the group consisting of: SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 38; SEQ ID NO: 39; SEQ ID NO: 40; SEQ ID NO: 41; SEQ ID NO: 42; SEQ ID NO: 43; SEQ ID NO: 44; SEQ ID NO: 45; SEQ ID NO: 46; SEQ ID NO: 47; SEQ ID NO: 48; SEQ ID NO: 49; and SEQ ID NO: 50.

43. A combination for use, a use, a method, a first antibody molecule for use, a pharmaceutical composition, or a kit according to claim 11, wherein the first antibody molecule comprises a variable light chain (VL) amino acid sequence selected from the group consisting of: SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 38; SEQ ID NO: 39; SEQ ID NO: 40; SEQ ID NO: 41; SEQ ID NO: 42; SEQ ID NO: 43; SEQ ID NO: 44; SEQ ID NO: 45; SEQ ID NO: 46; SEQ ID NO: 47; SEQ ID NO: 48; SEQ ID NO: 49; and SEQ ID NO: 50.

44. A combination for use, a use, a method, a first antibody molecule for use, a pharmaceutical composition, or a kit according to claim 12, wherein the first antibody molecule comprises a variable light chain (VL) amino acid sequence selected from the group consisting of: SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 38; SEQ ID NO: 39; SEQ ID NO: 40; SEQ ID NO: 41; SEQ ID NO: 42; SEQ ID NO: 43; SEQ ID NO: 44; SEQ ID NO: 45; SEQ ID NO: 46; SEQ ID NO: 47; SEQ ID NO: 48; SEQ ID NO: 49; and SEQ ID NO: 50.

45. A combination for use, a use, a method, a first antibody molecule for use, a pharmaceutical composition, or a kit according to claim 28, wherein the dose is at least 50% lower than the maximum tolerated therapeutic dose.
